SEQUENCE LISTING (27 sequences)

SEQ ID NO: 1 - DNA, 11 bp, Artificial Sequence
  Made in Lab - targeting sequence of antisense oligomer
tgctcatactc

SEQ ID NO: 2 - DNA, 11 bp, Artificial Sequence
  Made in Lab - targeting sequence of antisense oligomer
cttcgatagtg

SEQ ID NO: 3 - DNA, 11 bp, Artificial Sequence
  Made in Lab - targeting sequence of antisense oligomer
cgtttcattaa

SEQ ID NO: 4 - DNA, 11 bp, Artificial Sequence
  Made in Lab - targeting sequence of antisense oligomer
gtctattctcc

SEQ ID NO: 5 - DNA, 11 bp, Artificial Sequence
  Made in Lab - targeting sequence of antisense oligomer
gacatgtctat

SEQ ID NO: 6 - DNA, 11 bp, Artificial Sequence
  Made in Lab - targeting sequence of antisense oligomer
tggttctgcat

SEQ ID NO: 7 - DNA, 11 bp, Artificial Sequence
  Made in Lab - targeting sequence of antisense oligomer
tttatccattg

SEQ ID NO: 8 - DNA, 11 bp, Artificial Sequence
  Made in Lab - targeting sequence of antisense oligomer
gttcaaacata

SEQ ID NO: 9 - DNA, 11 bp, Artificial Sequence
  Made in Lab - targeting sequence of antisense oligomer
agtttctctcc

SEQ ID NO: 10 - DNA, 11 bp, Artificial Sequence
  Made in Lab - targeting sequence of antisense oligomer
ttcctgccata

SEQ ID NO: 11 - DNA, 11 bp, Artificial Sequence
  Made in Lab - targeting sequence of antisense oligomer
tttgatcatcg

SEQ ID NO: 12 - DNA, 11 bp, Artificial Sequence
  Made in Lab - targeting sequence of antisense oligomer
tctcagatggt

SEQ ID NO: 13 - protein, 12 aa, Artificial Sequence
  Made in Lab - Cell membrane penetrating peptide
  MOD_RES (2)..(2): Acp
  MOD_RES (5)..(5): Acp
  MOD_RES (8)..(8): Acp
  MOD_RES (11)..(11): Acp
RXRRXRRXRRXR

SEQ ID NO: 14 - protein, 10 aa, Artificial Sequence
  Made in Lab - Cell membrane penetrating peptide
RFFRFFRFFR

SEQ ID NO: 15 - protein, 14 aa, Artificial Sequence
  Made in Lab - Cell membrane penetrating peptide
  MOD_RES (2)..(2): Acp
  MOD_RES (5)..(5): Acp
  MOD_RES (8)..(8): Acp
  MOD_RES (11)..(11): Acp
  MOD_RES (13)..(13): Acp
  MOD_RES (14)..(14): Beta-alanine
RXRRXRRXRRXRXX

SEQ ID NO: 16 - protein, 12 aa, Artificial Sequence
  Made in Lab - Cell membrane penetrating peptide
  MOD_RES (11)..(11): Acp
  MOD_RES (12)..(12): bAla
RFFRFFRFFRXX

SEQ ID NO: 17 - protein, 12 aa, Artificial Sequence
  Made in Lab - Cell membrane penetrating peptide
RFRRFRRFRRFR

SEQ ID NO: 18 - protein, 12 aa, Artificial Sequence
  Made in Lab - Cell membrane penetrating peptide
RYRRYRRYRRYR

SEQ ID NO: 19 - protein, 12 aa, Artificial Sequence
  Made in Lab - Cell membrane penetrating peptide
RGRRGRRGRRGR

SEQ ID NO: 20 - protein, 14 aa, Artificial Sequence
  Made in Lab - Cell membrane penetrating peptide
  MOD_RES (13)..(13): Acp
  MOD_RES (14)..(14): bAla
RFRRFRRFRRFRXB

SEQ ID NO: 21 - protein, 14 aa, Artificial Sequence
  Made in Lab - Cell membrane penetrating peptide
  MOD_RES (13)..(13): Acp
  MOD_RES (14)..(14): bAla
RYRRYRRYRRYRXB

SEQ ID NO: 22 - protein, 14 aa, Artificial Sequence
  Made in Lab - Cell membrane penetrating peptide
  MOD_RES (13)..(13): Acp
  MOD_RES (14)..(14): bAla
RGRRGRRGRRGRXB

SEQ ID NO: 23 - protein, 12 aa, Artificial Sequence
  Made in Lab - Cell membrane penetrating peptide
  MOD_RES (11)..(11): Acp
  MOD_RES (12)..(12): bAla
RFFRFFRFFRXB

SEQ ID NO: 24 - protein, 11 aa, Artificial Sequence
  Made in Lab - Cell membrane penetrating peptide
RFFRFFRFFRG

SEQ ID NO: 25 - protein, 7 aa, Artificial Sequence
  Made in Lab - Cell membrane penetrating peptide
RRRRRRG

SEQ ID NO: 26 - protein, 13 aa, Artificial Sequence
  Made in Lab - Cell membrane penetrating peptide
  MOD_RES (2)..(2): Acp
  MOD_RES (5)..(5): Acp
  MOD_RES (8)..(8): Acp
  MOD_RES (11)..(11): Acp
RXRRXRRXRRXRG

SEQ ID NO: 27 - protein, 6 aa, Artificial Sequence
  Made in Lab - Cell membrane penetrating peptide
RRRRRR

(19) United States
(12) Patent Application Publication
(10) Pub. No.: US 2020/0283768 A1
(43) Pub. Date: Sep. 10, 2020

(54) ANTISENSE ANTIBACTERIAL COMPOUNDS AND METHODS

(71) Applicants: Board of Regents, The University of Texas System, Austin, OR (US); Oregon State University, Corvallis, OR (US)

(72) Inventors: Bruce L. GELLER, Corvallis, OR (US); David GREENBERG, Coppell, TX (US)

(73) Assignees: Board of Regents, The University of Texas System, Austin, TX (US); Oregon State University, Corvallis, OR (US)

(21) Appl. No.: 16/758,205

(22) PCT Filed: Oct. 19, 2018
(86) PCT No.: PCT/US2018/056621

§ 371 Date: Apr. 22, 2020

Related U.S. Application Data
(60) Provisional application No. 62/575,816, filed on Oct. 23, 2017.

(51) Int. Cl. C12N 15/113; A61P 31/04

(52) U.S. Cl. CPC C12N 15/113; A61P 31/04; C12N 2310/11; C12N 2310/351; C12N 2310/3233

(57) ABSTRACT

Provided are antisense oligomers targeted against bacterial mRNAs and other macromolecules associated with a biochemical pathway and/or cellular process, and related compositions and methods of using the oligomers and compositions to treat an infected mammalian subject, for example, as primary antimicrobials or as adjunctive therapies with classic antimicrobials.

This application claims benefit of priority to U.S. Provisional Application Ser. No. 62/575,816, filed Oct. 23, 2017, the entire contents of which are hereby incorporated by reference.

### I. STATEMENT REGARDING THE SEQUENCE LISTING

The Sequence Listing associated with this application is provided in text format in lieu of a paper copy and is hereby incorporated by reference into the specification. The name of the text file containing the Sequence Listing is UTFD_P3330WO_ST25.txt. The text file is about 8 KB, was created on Oct. 19, 2018, and is being submitted electronically via EFS-Web.

### II. BACKGROUND

### A. Technical Field

The present disclosure includes antisense oligomers targeted against bacterial mRNAs and other macromolecules involved in a biochemical pathway and/or cellular process, and related compositions and methods of using the oligomers and compositions to treat an infected mammalian subject, for example, as primary antimicrobials or as adjunctive therapies with classic antimicrobials.

### B. Description of the Related Art

Currently, there are several types of antibiotic compounds in use against bacterial pathogens and these compounds act through a variety of anti-bacterial mechanisms. For example, beta-lactam antibiotics, such as penicillin and cephalosporin, act to inhibit the final step in peptidoglycan synthesis. Glycopeptide antibiotics, including vancomycin and teichoplanin, inhibit both transglycosylation and transpeptidation of muramyl-pentapeptide, again interfering with peptidoglycan synthesis. Other well-known antibiotics include the quinolones, which inhibit bacterial DNA replication, inhibitors of bacterial RNA polymerase, such as rifampin, and inhibitors of enzymes in the pathway for production of tetrahydrofolate, including the sulfonamides.

Some classes of antibiotics act at the level of protein synthesis. Notable among these are the aminoglycosides, such as kanamycin and gentamicin. This class of compounds targets the bacterial 30S ribosome subunit, preventing the association with the 50S subunit to form functional ribosomes. Tetracyclines, another important class of antibiotics, also target the 30S ribosome subunit, acting by preventing alignment of aminoacylated tRNAs with the corresponding mRNA codon. Macrolides and lincosamides, another class of antibiotics, inhibit bacterial synthesis by binding to the 50S ribosome subunit, and inhibiting peptide elongation or preventing ribosome translocation.

Despite impressive successes in controlling or eliminating bacterial infections by antibiotics, the widespread use of antibiotics both in human medicine and as a feed supplement in poultry and livestock production has led to drug resistance in many pathogenic bacteria. Antibiotic resistance mechanisms can take a variety of forms. One of the major mechanisms of resistance to beta lactams, particularly in Gram-negative bacteria, is the enzyme beta-lactamase, which renders the antibiotic inactive by cleaving the lactam ring. Likewise, resistance to aminoglycosides often involves an enzyme capable of inactivating the antibiotic, in this case by adding a phosphoryl, adenyl, or acetyl group. Active efflux of antibiotics is another way that many bacteria develop resistance. Genes encoding efflux proteins, such as the tetA, tetG, tetL, and tetK genes for tetracycline efflux, have been identified. A bacterial target may develop resistance by altering the target of the drug. For example, the so-called penicillin binding proteins (PBPs) in many beta-lactam resistant bacteria are altered to inhibit the critical antibiotic binding to the target protein. Resistance to tetracycline may involve, in addition to enhanced efflux, the appearance of cytoplasmic proteins capable of competing with ribosomes for binding to the antibiotic. For those antibiotics that act by inhibiting a bacterial enzyme, such as for sulfonamides, point mutations in the target enzyme may confer resistance.

Klebsiella pneumoniae is found in the normal flora of the mouth, skin and intestines. However, it can cause severe lung problems if aspirated, and is a significant cause of hospital-acquired infections. Klebsiella can also cause infections in the urinary tract, lower biliary tract, and surgical wound sites, among other sides. The range of clinical diseases includes pneumonia, thrombophlebitis, urinary tract infection, cholecystitis, diarrhea, upper respiratory tract infection, wound infection, osteomyelitis, meningitis, and bacteremia, and septicemia. Klebsiella species are often resistant to multiple antibiotics. In fact, the spread of carbapenem-resistant Enterobacteriaceae (CRE) (including K. pneumoniae) has happened worldwide, including in the U.S. where carbapenemase-producing CRE has now been reported in most states.

Biofilm formation can also lead to antibiotic resistance, among other clinical difficulties. Typically, in situations where bacteria form a biofilm within an infected host, the infection turns out to be untreatable and can develop into a chronic state. Hallmarks of chronic biofilm-based infections not only include resistance to antibiotic treatments and many other conventional antimicrobial agents but also a capacity for evading host defenses. Therefore, strategies that prevent or breakdown biofilm would be of therapeutic interest and benefit.

The appearance of antibiotic resistance in many pathogenic bacteria, including cases involving multi-drug resistance (MDR), raises the fear of a post-antibiotic era in which many bacterial pathogens were simply untreatable by medical intervention. Thus, there is a need for antimicrobial agents that (i) are not subject to the principal types of antibiotic resistance currently hampering antibiotic treatment of bacterial infection, (ii) can be developed rapidly and with some reasonable degree of predictability as to target-bacteria specificity, (iii) are effective at low doses, and (iv) show few side effects.

### BRIEF SUMMARY

Embodiments of the present disclosure relate, in part, to the discovery that the antisense targeting of bacterial genes associated with biochemical pathways and/or cellular processes can reduce the ability of pathogenic Klebsiella bacteria to grow in vitro and in vivo, including antibiotic-resistant pathogenic bacteria and difficult-to-treat biofilms. The antisense oligomers described herein could thus find utility in the treatment of Klebsiella bacteria, for instance, as standalone therapies or in combination with antibiotics.

Embodiments of the present disclosure therefore include a substantially uncharged antisense morpholino oligomer, composed of morpholino subunits and phosphorus-containing intersubunit linkages joining a morpholino nitrogen of one subunit to a 5′-exocyclic carbon of an adjacent subunit, and having (a) about 10-40 nucleotide bases, and (b) a targeting sequence of sufficient length and complementarity to specifically hybridize to a bacterial mRNA target sequence that encodes a protein associated with a biochemical pathway and/or cellular process, as described herein. In some instances, the oligomer is conjugated to a cell-penetrating peptide (CPP).

In certain embodiments, the targeting sequence is selected from Tables 1A-B. In some embodiments, the oligomer is about 10-15 or about 11-12 nucleotide bases in length and has a targeting sequence selected from Tables 1A-B.

In certain embodiments, an antisense oligomer of the disclosure is of formula (I):

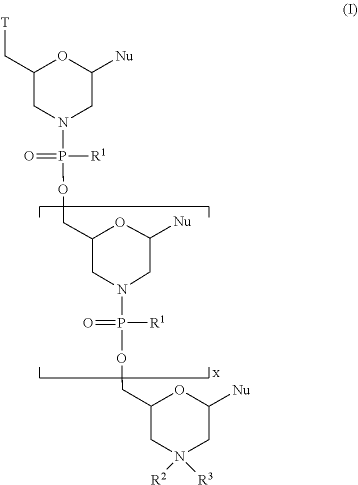

or a pharmaceutically acceptable salt thereof,

where each Nu is a nucleobase which taken together forms a targeting sequence;

X is an integer from 9 to 38;

T is selected from OH and a moiety of the formula:

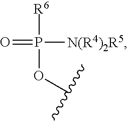

where each R⁴ is independently C₁-C₆ alkyl, and R⁵ is selected from an electron pair and H, and R⁶ is selected from OH, —N(R⁷)CH₂C(O)NH₂, and a moiety of the formula:

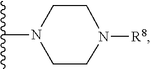

where:
R⁷ is selected from H and C₁-C₆ alkyl, and
R⁸ is selected from G, —C(O)—R⁹OH, acyl, trityl, and 4-methoxytrityl, where:
   R⁹ is of the formula —(O-alkyl)_{y}- wherein y is an integer from 3 to 10 and each of
   the y alkyl groups is independently selected from C₂-C₆ alkyl;
each instance of R¹ is —N(R¹⁰)₂R¹¹ wherein each R¹⁰ is independently C₁-C₆ alkyl, and R¹¹ is
selected from an electron pair and H;
R² is selected from H, G, acyl, trityl, 4-methoxytrityl, benzoyl, stearoyl, and a moiety of the formula:

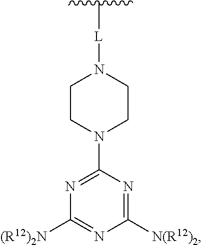

where L is selected from —C(O)(CH₂)₆C(O)— and —C(O)(CH₂)₂S₂(CH₂)₂C(O)—, and each R¹² is of the formula —(CH₂)₂OC(O)N(R¹⁴)₂ wherein each R¹⁴ is of the formula —(CH₂)₆NHC(═NH)NH₂; and
R³ is selected from an electron pair, H, and C₁-C₆ alkyl, wherein G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP, and —C(O)CH₂NH—CPP, or G is of the formula:

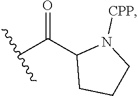

wherein the CPP is attached to the linker moiety by an amide bond at the CPP carboxy terminus, with the proviso that only one instance of G is present,

wherein the targeting sequence specifically hybridizes to a bacterial mRNA target sequence that encodes a protein associated with a biochemical pathway and/or cellular process, as described herein.

In various embodiments, the targeting sequence specifically hybridizes to a bacterial mRNA target sequence that encodes a protein associated with a biochemical pathway and/or cellular process.

In some embodiments, the target sequence comprises a translational start codon of the bacterial mRNA and/or a sequence within about 30 bases upstream or downstream of the translational start codon of the bacterial mRNA.

In some embodiments, the protein associated with a biochemical pathway and/or cellular process is a fatty acid biosynthesis protein. In certain embodiments, the fatty acid biosynthesis protein is an acyl carrier protein. In certain embodiments, the acyl carrier protein is encoded by acpP. In some embodiments, the fatty acid biosynthesis protein is an acyl carrier protein synthase. In some embodiments, the acyl carrier protein synthase is encoded by fabB.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In certain embodiments, the targeting sequence is set forth in SEQ ID NOS:1-3, comprises a fragment of at least 10 contiguous nucleotides of SEQ ID NOS: 1-3, or comprises a variant having at least 80% sequence identity to SEQ ID NOS: 1-3, where thymine bases (T) are optionally uracil bases (U). In specific embodiments, the antisense morpholino oligomer is selected from Table 2A.

In some embodiments, the protein associated with a biochemical pathway and/or cellular process is a peptidoglycan biosynthesis protein. In certain embodiments, the peptidoglycan biosynthesis protein is a UDP-N-acetylglucosamine 1-carboxyvinyltransferase. In particular embodiments, the UDP-N-acetylglucosamine 1-carboxyvinyltransferase is encoded by murA.

In certain embodiments, the protein associated with a biochemical pathway and/or cellular process is a ribosomal protein. In some embodiments, the ribosomal protein is a 50S ribosomal protein L28. In certain embodiments, the 50S ribosomal protein L28 is encoded by rpmB. In some embodiments, the ribosomal protein is a 30S ribosomal protein. In some embodiments, the 30S ribosomal protein is encoded by rpsJ.

In certain embodiments, the protein associated with a biochemical pathway and/or cellular process is a cell division protein. In particular embodiments, the cell division protein is a protein that assembles into a ring at the future site of the septum of bacterial cell division. In some embodiments, the protein that assembles into a ring at the future site of the septum of bacterial cell division is encoded by ftsZ.

In certain embodiments, the protein associated with a biochemical pathway and/or cellular process is a DNA or chromosomal replication protein. In some embodiments, DNA or chromosomal replication protein is a helicase. In some embodiments, the helicase is encoded by dnaB

In specific embodiments, the targeting sequence is set forth in SEQ ID NOS:4-11, comprises a fragment of at least 10 contiguous nucleotides of SEQ ID NOS: 4-11, or comprises a variant having at least 80% sequence identity to SEQ ID NOS: 4-11, where thymine bases (T) are optionally uracil bases (U). In specific embodiments, the antisense morpholino oligomer is selected from Table 2B.

Also included are pharmaceutical compositions, comprising a pharmaceutically acceptable carrier and an antisense oligomer described herein. Some pharmaceutical compositions further comprising an antimicrobial agent as described herein. Illustrative examples of antimicrobial agents include tobramycin, meropenem, and colistin, and combinations thereof.

Some embodiments include methods of reducing expression and activity of a protein associated with a biochemical pathway and/or cellular process in a bacterium, comprising contacting the bacterium with an antisense oligomer and/or a pharmaceutical composition described herein.

In certain embodiments, the bacterium is in a subject, and the method comprises administering the antisense oligomer to the subject. In some embodiments, the bacterium is from the genera Klebsiella. In particular embodiments, the bacterium is an antibiotic-resistant strain of Klebsiella. In some embodiments, the bacterium is a multi-drug resistant (MDR) strain of Klebsiella. In specific embodiments, the bacterium is Klebsiella pneumonia, for example, selected from Table 3. In specific embodiments, the subject is suspected of having or has a urinary tract infection, and/or is subject is suspected of having a Klebsiella spp. or E. coli infection. In such cases the antisense morpholino oligomer employed may comprise SEQ ID NO: 1 or 2.

In certain embodiments, the subject in need thereof is immunocompromised. In some embodiments, the subject has underlying lung disease, such as cystic fibrosis (CF) and/or chronic granulomatous disease (CGD). In some embodiments, the bacterium is in the lung(s) of the subject, for example, as a bacterial lung infection. In some embodiments, administration of the antisense oligomer reduces bacterial lung burden by at least about 10%.

In certain embodiments, the bacterium has formed or is at risk for forming a biofilm in the subject. In certain embodiments, administration of the antisense oligomer reduces biofilm formation or existing biofilm by at least about 10%.

Some methods comprise administering the oligomer separately or concurrently with an antimicrobial agent, optionally where administration of the oligomer increases susceptibility of the bacterium to the antimicrobial agent.

In certain embodiments, the antimicrobial agent is selected from one or more of a 3-lactam antibiotic, an aminoglycoside antibiotic, and a polymyxin.

In some embodiments, the pβ-lactam antibiotic is selected from at least one of carbapenems, penicillin derivatives (penams), cephalosporins (cephems), and monobactams.

In particular embodiments, the carbapenem is selected from one or more of meropenem, imipenem, ertapenem, doripenem, panipenem, biapenem, razupenem, tebipenem, lenapenem, and tomopenem. In specific embodiments, the carbapenem is meropenem.

In certain embodiments, the aminoglycoside antibiotic is selected from one or more of tobramycin, gentamicin, kanamycin a, amikacin, dibekacin, sisomicin, netilmicin, neomycin B, neomycin C, neomycin E (paromomycin), and streptomycin. In specific embodiments, the aminoglycoside antibiotic is tobramycin.

In certain embodiments, the polymyxin is selected from one or more of colistin (polymyxin E), polysporin, neosporin, or polymyxin B. In specific embodiments, the polymyxin is colistin.

In some embodiments, the β-lactam antibiotic is selected from at least one of meropenem, imipenem, ertapenem, doripenem, panipenem, biapenem, razupenem, tebipenem, lenapenem, tomopenem, cephalosporins (cephems), penicillin, penicillin derivatives (penams) and ampicillin.

In certain embodiments, the aminoglycoside antibiotic is selected from at least one of tobramycin, gentamicin, kanamycin a, amikacin, dibekacin, sisomicin, netilmicin, neomycin B, neomycin C, neomycin E (paromomycin), and streptomycin.

In some embodiments, the tetracycline antibiotic is selected from at least one of tetracycline, chlortetracycline, oxytetracycline, demeclocycline, lymecycline, meclocycline, methacycline, minocycline, rolitetracycline, and doxycyline.

In particular embodiments, the β-lactam antibiotic is selected from at least one of carbapenems, penicillin derivatives (penams), cephalosporins (cephems), and monobactams.

In some embodiments, the oligomer reduces the minimum inhibitory concentration (MIC) of the antimicrobial agent against the bacterium by at least about 10% relative to the antimicrobial agent alone.

In some embodiments, the oligomer increases the susceptibility of the bacterium to the antimicrobial agent by at least about 10% relative to the antimicrobial agent alone.

In certain embodiments, the combination of oligomer and the antimicrobial agent synergistically increases the susceptibility of the bacterium to the antibiotic relative to the oligomer and/or the microbial agent alone.

In certain embodiments, the antimicrobial agent and the antisense oligomer are administered separately. In various embodiments, the antimicrobial agent and the antisense oligomer are administered sequentially. In some embodiments, the antimicrobial agent and the antisense oligomer are administered concurrently.

It is contemplated that any method or composition described herein can be implemented with respect to any other method or composition described herein. Moreover, it is clearly contemplated that embodiments may be combined with one another, to the extent they are compatible.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

It is specifically contemplated that any embodiments described in the Examples section are included as an embodiment of the disclosure.

Following the long-standing patent law convention, the words “a” and “an,” when used in conjunction with the word “comprising” in the claims or specification, denotes one or more, unless specifically noted.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIGS. 1A-H shows an exemplary morpholino oligomer structure with a phosphorodiamidate linkage. FIGS. 1B-E show the repeating subunit segment of exemplary morpholino oligomers, designated FIG. 1B through FIG. 1E. FIGS. 1F-H show exemplary peptide PMO conjugates structures used in the exemplary PPMOs.

FIGS. 2A-B show the results of Minimal Bactericidal Concentration (MBC) assays. Samples from cultures without visible growth in the MIC assay were diluted and spread on LB agar petri dishes. After overnight growth on the petri dishes, colonies were enumerated. 2×, 1× and 0.5× refer to multiples of the MIC value of each AcpP PPMO. Error bars indicate standard deviation, n=3 for AcpP PPMOs and n=1 for Scr PPMOs.

FIGS. 3A-C show the results of biofilm reduction assays. Biofilms were established (24 h) and then treated with (a, b) 8, 16, or 32 μM AcpP PPMO (0276 or 0802) or Scr PPMO (1073 or 1344) in growth medium. After 48 h, (FIG. 3A) biofilm mass and (FIG. 3B) viable cells within the biofilm were measured. N=6 (0276 and Scr 1073) and =4 (0802 and Scr 1344). (FIG. 3C) Same as FIGS. 3A and 3B, except that established biofilms were treated with 32 μM PPMO (0276 or 1073) in PBS instead of growth medium. N=9 to 12. Bars indicate means and standard deviations.

FIGS. 4A-4F show MBEC biofilm reduction assays and confocal microscopy. Established biofilms on MBEC pegs (24 h) were treated with one dose of AcpP-0802 or Scr-1344 PPMO, incubated for 24 h, then quantitated by (FIG. 4A) crystal violet assay and (FIG. 4B) colony count, with corresponding confocal microscopy of biofilm with (FIG. 4C) no treatment, at a 16 μM dose of (FIG. 4D) Scr PPMO or (FIG. 4E) AcpP PPMO, and at a (FIG. 4F) 2 μM dose of AcpP PPMO. Dashed lines represent limit of detection. Significance of decrease in biofilm formation compared to Scr PPMO is given as ***p<0.0001. Statistics were calculated using two-way ANOVA with Tukey's multiple comparisons test.

FIGS. 5A-C show the results of in vivo infectious challenge and treatment with R₆G-AcpP PPMO (0802). Mice were infected with K. pneumoniae NR15410 and treated at 0, 24, and 48 h post-infection with 600 μg (n=9), 200 μg (n=13), 67 μg (n=6), or 12.5 μg (n=3) AcpP-PPMO, 600 μg scrambled PPMO (Scr) (n=9), or PBS (n=14). (a) Survival, (b) body temperature, and (c) weight were recorded. For Kaplan-Meyer survival curves, **p<0.0001 *p=0.0016 by log-rank (Mantel-Cox) test.

FIGS. 6A-C show the results of therapeutic (delayed) treatments. Groups of mice were infected intranasally with K. pneumoniae NR15410. Initial treatment with 30 mg/kg R6G-AcpP PPMO (0802) was given at 0, 8, 24, or 48 hours post-infection, and then again at 24 hour intervals for 3 days. (FIG. 6A) Survival, (FIG. 6B) body temperature, and (FIG. 6C) weight was monitored.

FIG. 7 shows the results of testing of bacterial lung burden. Mice were infected and treated once at 6 hours post-infection. Lungs were removed at 24 hours post-infection, homogenized, and then spread on LB agar petri dishes, which were incubated 18 hours at 37° C. Bacterial colonies on petri dishes were enumerated. * Indicates statistically significant difference (p<0.0001).

FIG. 8 shows a MIC heatmap of PPMOs targeted against essential genes of K. pneumoniae.

FIGS. 9A-D show the results of in vivo infectious challenge and treatment with R₆G-AcpP PPMO (0802). Mice were infected with E. coli CVB-1 and treated with 5 doses of treatment or placebo out to 24 hours post-infection with a 1 mg dose, 300 μg dose, 100 μg dose, 1 mg scrambled placebo PPMO or PBS. (FIG. 9A) shows body temperature, (FIG. 9B) shows body weight, (FIG. 9C) shows log cfu/ml blood, and (FIG. 9D) shows survival. 1 mg and 300 μg dosing showed significant increases in survival in this aggressive sepsis model of infection.

### DETAILED DESCRIPTION

### I. Definitions

Unless defined otherwise, all technical and scientific terms used herein have the same meaning as commonly understood by those of ordinary skill in the art. Although any methods and materials similar or equivalent to those described herein can be used in the practice or testing of the present disclosure, preferred methods and materials are described. For the purposes of the present disclosure, the following terms are defined below.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “contacting a cell”, “introducing” or “delivering” include delivery of the oligomers described herein into a cell by methods routine in the art, e.g., transfection (e.g., liposome, calcium-phosphate, polyethyleneimine), electroporation (e.g., nucleofection), microinjection), transformation, and administration.

The terms “cell penetrating peptide” (CPP) or “a peptide moiety which enhances cellular uptake” are used interchangeably and refer to cationic cell penetrating peptides, also called “transport peptides”, “carrier peptides”, or “peptide transduction domains”. In some aspects, the peptides have the capability of inducing cell penetration within about or at least about 30%, 40%, 50%, 60%, 70%, 80%, 90%, or 100% of cells of a given population and/or allow macromolecular translocation to or within multiple tissues in vivo upon systemic administration. Particular examples of CPPs include “arginine-rich peptides.” CPPs are well-known in the art and are disclosed, for example, in U.S. Application No. 2010/0016215 and International Patent Application Publication Nos. WO 2004/097017, WO 2009/005793, and WO 2012/150960, all of which are incorporated by reference in their entirety.

“An electron pair” refers to a valence pair of electrons that are not bonded or shared with other atoms.

“Homology” refers to the percentage number of amino acids that are identical or constitute conservative substitutions. Homology may be determined using sequence comparison programs such as GAP (Deveraux et al., 1984, Nucleic Acids Research 12, 387-395) or BLAST. In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated polynucleotide” or “isolated oligomer,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

The term “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or antisense compounds or compositions to produce or cause a greater physiological response (i.e., downstream effects) in a cell or a subject relative to the response caused by either no antisense compound or a control compound. Relevant physiological or cellular responses (in vivo or in vitro) will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and ranges between and above 1), e.g., 1.5, 1.6, 1.7. 1.8) the amount produced by no antisense compound (the absence of an agent) or a control compound. The term “reduce” or “inhibit” may relate generally to the ability of one or more antisense compounds or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in bacterial cell growth, reductions in the minimum inhibitory concentration (MIC) of an antimicrobial agent, and others. A “decrease” in a response may be “statistically significant” as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers and ranges in between.

As used herein, an “antisense oligomer,” “oligomer,” or “oligomer” refers to a linear sequence of nucleotides, or nucleotide analogs, which allows the nucleobase to hybridize to a target sequence in an RNA by Watson-Crick base pairing, to form an oligomer:RNA heteroduplex within the target sequence. The terms “antisense oligomer,” “antisense oligomer,” “oligomer,” and “compound” may be used interchangeably to refer to an oligomer. The cyclic subunits may be based on ribose or another pentose sugar or, in certain embodiments, a morpholino group (see description of morpholino oligomers below).

The term “oligomer,” “oligomer,” or “antisense oligomer” also encompasses an oligomer having one or more additional moieties conjugated to the oligomer, e.g., at its 3′- or 5′-end, such as a polyethylene glycol moiety or other hydrophilic polymer, e.g., one having 10-100 monomeric subunits, which may be useful in enhancing solubility, or a moiety such as a lipid or peptide moiety that is effective to enhance the uptake of the compound into target bacterial cells and/or enhance the activity of the compound within the cell, e.g., enhance its binding to a target polynucleotide.

A “nuclease-resistant” oligomers refers to one whose backbone is substantially resistant to nuclease cleavage, in non-hybridized or hybridized form; by common extracellular and intracellular nucleases in the body or in a bacterial cell (for example, by exonucleases such as 3′-exonucleases, endonucleases, RNase H); that is, the oligomer shows little or no nuclease cleavage under normal nuclease conditions to which the oligomer is exposed. A “nuclease-resistant heteroduplex” refers to a heteroduplex formed by the binding of an antisense oligomer to its complementary target, such that the heteroduplex is substantially resistant to in vivo degradation by intracellular and extracellular nucleases, which are capable of cutting double-stranded RNA/RNA or RNA/DNA complexes. A “heteroduplex” refers to a duplex between an antisense oligomer and the complementary portion of a target RNA.

As used herein, “nucleobase” (Nu), “base pairing moiety” or “base” are used interchangeably to refer to a purine or pyrimidine base found in native DNA or RNA (uracil, thymine, adenine, cytosine, and guanine), as well as analogs of the naturally occurring purines and pyrimidines, that confer improved properties, such as binding affinity to the oligomer. Exemplary analogs include hypoxanthine (the base component of the nucleoside inosine); 2, 6-diaminopurine; 5-methyl cytosine; C5-propynyl-modified pyrimidines; 9-(aminoethoxy)phenoxazine (G-clamp) and the like.

A nucleobase covalently linked to a ribose, sugar analog or morpholino comprises a nucleoside. “Nucleotides” are composed of a nucleoside together with one phosphate group. The phosphate groups covalently link adjacent nucleotides to one another to form an oligomer.

An oligomer “specifically hybridizes” to a target sequence if the oligomer hybridizes to the target under physiological conditions, with a Tm substantially greater than 40° C. or 45° C., preferably at least 50° C., and typically 60° C.-80° C. or higher. Such hybridization preferably corresponds to stringent hybridization conditions. At a given ionic strength and pH, the Tm is the temperature at which 50% of a target sequence hybridizes to a complementary polynucleotide. Such hybridization may occur with “near” or “substantial” complementarity of the antisense oligomer to the target sequence, as well as with exact complementarity.

As used herein, “sufficient length” includes an antisense oligomer that is complementary to at least about 8, more typically about 8-10, 8-11, 8-12, 8-13, 8-14, 8-15, 8-16, 8-17, 8-18, 8-19, 8-20, 8-30, 8-40, or 10-11, 10-12, 10-13, 10-14, 10-15, 10-16, 10-17, 10-18, 10-19, 10-20, 10-30, 10-40 (including all integers and ranges in between) contiguous or non-contiguous nucleobases in a region of a bacterial mRNA target sequence. An antisense oligomer of sufficient length has at least a minimal number of nucleotides to be capable of specifically hybridizing to a region of the bacterial mRNA. Preferably an oligomer of sufficient length is from 8 to 30 nucleotides in length, for example, about 10-20 nucleotides in length.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

A “subject” or a “subject in need thereof” includes a mammalian subject such as a human subject.

The terms “TEG,” “EG3,” or “triethylene glycol tail” refer to triethylene glycol moieties conjugated to the oligomer, e.g., at its 3′- or 5′-end. For example, in some embodiments, “TEG” includes, for example, wherein T of the compound of formula (I), (II), or (III) is of the formula:

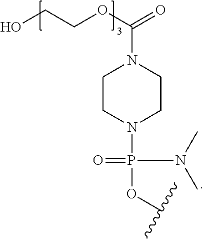

The term “pip-PDA” refers to a 5′ terminal piperazine-phosphorodiamidate moiety that connects a G group, where the G group comprises a cell-penetrating peptide (CPP) and linker moiety further discussed below, to the 5′end of the oligomer by way of an amide bond between the G group linker and the piperazinyl nitrogen. For example, in some embodiments, “pip-PDA” includes wherein T of the compound of formula (I) or (II) is of the formula:

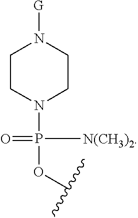

The term “target sequence” refers to a portion of the target RNA, for example, a bacterial mRNA, against which the antisense oligomer is directed, that is, the sequence to which the oligomer will hybridize by Watson-Crick base pairing of a complementary sequence. In certain embodiments, the target sequence may be a contiguous region of the translation initiation region of a bacterial mRNA.

The term “targeting sequence” or “antisense targeting sequence” refers to the sequence in an oligomer that is complementary or substantially complementary to the target sequence in the RNA, e.g., the bacterial mRNA. The entire sequence, or only a portion, of the antisense compound may be complementary to the target sequence. For example, in an oligomer of about 10-30 bases, about 6, 7, 8, 9, 10, 11, 12, 13, 14, 15, 16, 17, 18, 19, 20, 21, 22, 23, 24, 25, 26, 27, 28, or 29 of the bases may be targeting sequences that are complementary to the target region. Typically, the targeting sequence is formed of contiguous bases, but may alternatively be formed of non-contiguous sequences that when placed together, e.g., from opposite ends of the oligomer, constitute sequence that spans the target sequence.

A “targeting sequence” may have “near” or “substantial” complementarity to the target sequence and still function for the purpose of the present disclosure, that is, still be “complementary.” Preferably, the oligomer analog compounds employed in the present disclosure have at most one mismatch with the target sequence out of 10 nucleotides, and preferably at most one mismatch out of 20. Alternatively, the antisense oligomers employed have at least 90% sequence homology, and preferably at least 95% sequence homology, with the exemplary targeting sequences as designated herein.

As used herein, the term “quantifying”, “quantification” or other related words refer to determining the quantity, mass, or concentration in a unit volume, of a nucleic acid, polynucleotide, oligomer, peptide, polypeptide, or protein.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

### II. Bacterial Targeting Sequences

Certain embodiments relate to antisense oligomers, and related compositions and methods, which are of sufficient length and complementarity to specifically hybridize to a bacterial mRNA target sequence that encodes a gene in a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

Particular examples of genes in biochemical pathways and cellular processes include: rpsJ and rpmB (ribosomal proteins); lpxC (lipopolysaccharide biosynthesis); murA (formerly known as murZ) (peptidoglycan biosynthesis); acpP and fabB (fatty acid biosynthesis); ftsZ (cell division); and dnaB (chromosomal and DNA replication). In some embodiments, the target sequence that encodes the gene is from Klebsiella, e.g., Klebsiella pneumoniae.

In some embodiments, the bacterial target is a gene or protein that is associated with biosynthesis of fatty acids. General examples of proteins associated with fatty acid biosynthesis include: acyl carrier protein (ACP), such as AcpP, that plays an essential role in stabilizing and shuttling the intermediate fatty acid chain to each of the enzymes in the fatty acid synthase complex; acyl carrier protein synthase (AcpS), an enzyme that transfers the 4′-phosphopantetheine prosthetic group to apo-ACP to form the functional holo-ACP; acetyl-CoA carboxylase, an enzyme composed of four proteins that catalyzes the conversion of acetyl-CoA to malonyl-CoA in the first committed step of fatty acid biosynthesis: AccA (carboxyltransferase alpha subunit catalyzing the transfer of the carboxyl group from biotin to acetyl-CoA to form malonyl-CoA), AccB (biotin carboxyl carrier protein, BCCP, carrying the biotin prosthetic group covalently attached to a lysine residue proximal to the carboxyl terminus), AccC (biotin carboxylase catalyzing the carboxylation of protein bound biotin with bicarbonate), AccD (carboxyltransferase beta subunit catalyzing the transfer of the carboxyl group from biotin to acetyl-CoA to form malonyl-CoA); fatty acid biosynthesis (Fab) enzymes, such as FabA, FabB, FabI, FabF, FabD, FabH, FabG and FabZ, that each catalyze either elongation or tailoring steps on the growing fatty acid chain. Particular examples of genes associated with fatty acid biosynthesis include acpP, and the acyl carrier protein synthase fabB.

Specific embodiment therefore relate to antisense oligomers, and related compositions and methods, which are of sufficient length and complementarity to specifically hybridize to an mRNA target sequence of a bacterial acpP gene, which encodes an acyl carrier protein (ACP). In some embodiments, the acpP gene is from Klebsiella, e.g., Klebsiella pneumoniae.

Certain embodiment relate to antisense oligomers, and related compositions and methods, which are of sufficient length and complementarity to specifically hybridize to an mRNA target sequence of a bacterial fabB gene. In some embodiments, the fabB gene is from Klebsiella, e.g., Klebsiella pneumoniae.

The bacterial cell wall peptidoglycan is an essential cellular component involved in the maintenance of shape and protection from osmotic shock lysis. Typically, peptidoglycan is assembled from a basic building block composed of N-acetylglucosamine (GlcNAc) and N-acetylmuramic acid with an attached pentapeptide. In some embodiments, the bacterial target is a gene or protein that is associated with peptidoglycan biosynthesis. A particular example of a gene associated with peptidoglycan biosynthesis include murA (formerly known as murZ), which encodes a UDP-N-acetylglucosamine 1-carboxyvinyltransferase, which catalyzes the first committed step of peptidoglycan biosynthesis. The enzyme catalyzes the transfer of enolpyruvate from phosphoenolpyruvate to the 3-OH of UDP-N-acetylglucosamine. In some embodiments, the murA gene is from Klebsiella, e.g., Klebsiella pneumoniae.

The ribosome is crucial for translation of mRNA molecules into proteins. In some embodiments, the bacterial target is a gene or protein that is associated with ribosomal proteins. A particular example of a gene associated with ribosomal proteins is rpmB, a 50S ribosomal protein L28 essential for ribosome assembly and translation. Another example of a gene associated with ribosomal proteins is rpsJ, a 30S ribosomal protein. In some embodiments, the rpmB or rpsJ gene is from Klebsiella, e.g., Klebsiella pneumoniae.

In some embodiments, the bacterial target is a gene or protein that is associated with cell division. A particular example of a gene associated with cell division includes a ftsZ gene, which encodes a protein that assembles into a ring at the future site of the septum of bacterial cell division. This is a prokaryotic homologue to the eukaryotic protein tubulin. In some embodiments, the ftsZ gene is from Klebsiella, e.g., Klebsiella pneumoniae.

In some embodiments, the bacterial target is a gene or protein that is associated with DNA or chromosomal replication. One example of a gene associated with DNA or chromosomal replication is dnaB, which encodes a helicase. In some embodiments, the dnaB gene is from Klebsiella, e.g., Klebsiella pneumoniae.

In some embodiments, the bacterial target is a gene or protein that is associated with lipopolysaccharide biosynthesis. One example of a gene associated with lipopolysaccharide biosynthesis is lpxC, which encodes an N-acetylglucosamine deacetylase. In some embodiments, the lpxC, gene is from Klebsiella, e.g., Klebsiella pneumoniae.

In certain embodiments, the target sequence contains all or a portion (e.g., 1 or 2 nucleotides) of a translational start codon of the bacterial mRNA. In some embodiments, the target sequence contains a sequence that is about or within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 bases upstream or downstream of a translational start codon of the bacterial mRNA target sequence, including common and alternative start codons (e.g., AUG, GUG, UUG, AUU, CUG). For example, in particular embodiments, the 5′-end of the target sequence is the adenine, uracil, or guanine nucleotide (respectively) in an AUG start codon of the bacterial mRNA. In some embodiments, the 5′-end of the target sequence is the guanine, uracil, or guanine nucleotide (respectively) in a GUG start codon of the bacterial mRNA. In some embodiments, the 5′-end of the target sequence is the uracil, uracil, or guanine nucleotide (respectively) in a UUG start codon of the bacterial mRNA. In some embodiments, the 5′-end or 3-end of the target sequence begins at residue 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 downstream of the last (third) nucleotide of a translational start codon of the bacterial mRNA. In some embodiments, the 5′-end or 3-end of the target sequence begins at residue 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 upstream of the first nucleotide of a translational start codon of the bacterial mRNA.

Thus, in certain embodiments, antisense targeting sequences are designed to hybridize to a region of one or more of the target genes described herein. Selected antisense targeting sequences can be made shorter, e.g., about 8, 9, 10, 11, 12, 13, 14, or 15 bases, or longer, e.g., about 20, 30, or 40 bases, and include a small number of mismatches, as long as the sequence is sufficiently complementary to reduce transcription or translation upon hybridization to the target sequence, and optionally forms with the RNA a heteroduplex having a Tm of 45° C. or greater.

In certain embodiments, the degree of complementarity between the target sequence and antisense targeting sequence is sufficient to form a stable duplex. The region of complementarity of the antisense oligomers with the target RNA sequence may be as short as 8-9 bases, 8-10 bases, 8-11 bases, 8-12 bases, 10-11 bases, 10-12 bases, but can be 12-15 bases or more, e.g., 10-40 bases, 12-30 bases, 12-25 bases, 15-25 bases, 12-20 bases, or 15-20 bases, including all integers in between these ranges. An antisense oligomer of about 10-15 bases is generally long enough to have a unique complementary sequence. In certain embodiments, a minimum length of complementary bases may be required to achieve the requisite binding Tm, as discussed herein.

In certain embodiments, oligomers as long as 40 bases may be suitable, where at least a minimum number of bases, e.g., 10-12 bases, are complementary to the target sequence. In general, however, facilitated or active uptake in cells is optimized at oligomer lengths of less than about 30 or less than about 20 bases. Included are antisense oligomers that consist of about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases, in which at least about 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous or non-contiguous bases are complementary to a target gene described herein.

In certain embodiments, antisense oligomers may be 100% complementary to the target sequence, or may include mismatches, e.g., to accommodate variants, as long as a heteroduplex formed between the oligomer and target sequence is sufficiently stable to withstand the action of cellular nucleases and other modes of degradation which may occur in vivo, and reduce expression of the targeted mRNA. Hence, certain oligomers may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligomer and the target sequence. Oligomer backbones that are less susceptible to cleavage by nucleases are discussed herein. Mismatches, if present, are typically less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the target sequence, it is effective to stably and specifically bind to the target sequence, for example, such that translation of the target RNA is reduced.

The stability of the duplex formed between an oligomer and a target sequence is a function of the binding Tm and the susceptibility of the duplex to cellular enzymatic cleavage. The Tm of an oligomer with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligomer Hybridization Techniques, Methods Enzymol. Vol. 154 pp. 94-107. In certain embodiments, antisense oligomers may have a binding Tm, with respect to a complementary-sequence RNA, of greater than body temperature and preferably greater than about 45° C. or 50° C. Tm's in the range 60-80° C. or greater are also included. According to well-known principles, the Tm of an oligomer, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer.

Tables 1A-B below show exemplary targeting sequences (in a 5′-to-3′ orientation) of the antisense oligomers described herein. Table 1C provides scramble control sequence(s).

TABLE 1A
Exemplary Fatty Acid Biosynthesis-Associated
Targeting Sequences

| Target | Targeting Sequence | |
|---|---|---|
| Gene | (TS)* | SEQ ID NO: |
| acpP | TGCTCATACTC | 1 |
| acpP | CTTCGATAGTG | 2 |
| fabB | CGTTTCATTAA | 3 |

TABLE 1B
Exemplary targeting sequences associated with
other biochemical pathways and/or cellular
processes

| Target | Targeting Sequence | |
|---|---|---|
| Gene | (TS)* | SEQ ID NO: |
| rpmB | GTCTATTCTCC | 4 |
| rpmB | GACATGTCTAT | 5 |
| rpsJ | TGGTTCTGCAT | 6 |
| murA | TTTATCCATTG | 7 |
| ftsZ | GTTCAAACATA | 8 |
| ftsZ | AGTTTCTCTCC | 9 |
| dnaB | TTCCTGCCATA | 10 |
| lpxC | TTTGATCATCG | 11 |

TABLE 1C
Exemplary Scramble Control Sequences

| Target Gene | Targeting Sequence (TS)* | SEQ ID NO: |
|---|---|---|
| scramble | TCTCAGATGGT | 12 |

Certain antisense oligomers thus comprise, consist, or consist essentially of a targeting sequence in Tables 1A-B (e.g., SEQ ID NOS: 1-11) or a variant or contiguous or non-contiguous portion(s) thereof. For instance, certain antisense oligomers comprise about or at least about 6, 7, 8, 9, 10, 11, 12, 13, 14, 15, 16, 17, 18, 19, 20, 21, 22, 23, 24, 25, 26, or 27 contiguous or non-contiguous nucleotides of any of the targeting sequences in Tables 1A-B (e.g., SEQ ID NOS: 1-11). For non-contiguous portions, intervening nucleotides can be deleted or substituted with a different nucleotide, or intervening nucleotides can be added. Additional examples of variants include oligomers having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of the targeting sequences in Tables 1A-B (e.g., SEQ ID NOS: 1-11).

The activity of antisense oligomers and variants thereof can be assayed according to routine techniques in the art (see, e.g., the Examples).

### III. Antisense Oligomer Compounds

The antisense oligomers typically comprises a base sequence of sufficient length and complementarity to specifically hybridize to a bacterial mRNA target sequence that encodes a protein associated with a biochemical pathway and/or cellular process and thereby reduce expression (e.g., translation) of the protein, and thereby inhibit its interaction with other macromolecules. This requirement is optionally met when the oligomer compound has the ability to be actively taken up by bacterial cells, and once taken up, form a stable duplex (or heteroduplex) with the target mRNA, optionally with a Tm greater than about 40° C. or 45° C.

A. Antisense Oligomer Chemical Features

In certain embodiments, the backbone of the antisense oligomer is substantially uncharged, and is optionally recognized as a substrate for active or facilitated transport across a cell wall and/or cell membrane. The ability of the oligomer to form a stable duplex with the target RNA may also relate to other features of the backbone, including the length and degree of complementarity of the antisense oligomer with respect to the target, the ratio of G:C to A:T base matches, and the positions of any mismatched bases. The ability of the antisense oligomer to resist cellular nucleases may promote survival and ultimate delivery of the agent to the cell. Exemplary antisense oligomer targeting sequences are listed in Tables 1A-B (supra).

In certain embodiments, the antisense oligomer is a morpholino-based oligomer, for example, a phosphorodiamidate morpholino oligomer (PMO). Morpholino-based oligomers refer to an oligomer comprising morpholino subunits supporting a nucleobase and, instead of a ribose, contains a morpholine ring. Exemplary internucleoside linkages include, for example, phosphoramidate or phosphorodiamidate internucleoside linkages joining the morpholine ring nitrogen of one morpholino subunit to the 4′ exocyclic carbon of an adjacent morpholino subunit. Each morpholino subunit comprises a purine or pyrimidine nucleobase effective to bind, by base-specific hydrogen bonding, to a base in an oligomer.

Morpholino-based oligomers (including antisense oligomers) are detailed, for example, in U.S. Pat. Nos. 5,698,685; 5,217,866; 5,142,047; 5,034,506; 5,166,315; 5,185,444; 5,521,063; 5,506,337 and pending U.S. patent application Ser. Nos. 12/271,036; 12/271,040; and PCT Publication No. WO/2009/064471 and WO/2012/043730 and Summerton et al. 1997, Antisense and Nucleic Acid Drug Development, 7, 187-195, which are hereby incorporated by reference in their entirety.

Within the oligomer structure, the phosphate groups are commonly referred to as forming the “internucleoside linkages” of the oligomer. The naturally occurring internucleoside linkage of RNA and DNA is a 3′ to 5′ phosphodiester linkage. A “phosphoramidate” group comprises phosphorus having three attached oxygen atoms and one attached nitrogen atom, while a “phosphorodiamidate” group comprises phosphorus having two attached oxygen atoms and two attached nitrogen atoms. In the uncharged or the cationic internucleoside linkages of the morpholino-based oligomers described herein, one nitrogen is always pendant to the linkage chain. The second nitrogen, in a phosphorodiamidate linkage, is typically the ring nitrogen in a morpholine ring structure.

In particular embodiments, the morpholino subunits are joined by phosphorous-containing intersubunit linkages in accordance with the structure:

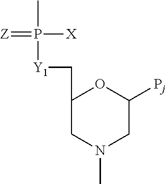

where Y₁=oxygen (O) or sulfur, nitrogen, or carbon; Z=oxygen or sulfur, preferably oxygen; Pj is a purine or pyrimidine base-pairing moiety effective to bind, by base-specific hydrogen bonding, to a base in a polynucleotide, and X is —NRR′ where R and R′ are the same or different and are either H or alkyl. In particular embodiments, X is —NRR′, where R and R′ are the same or different and are either H or methyl.

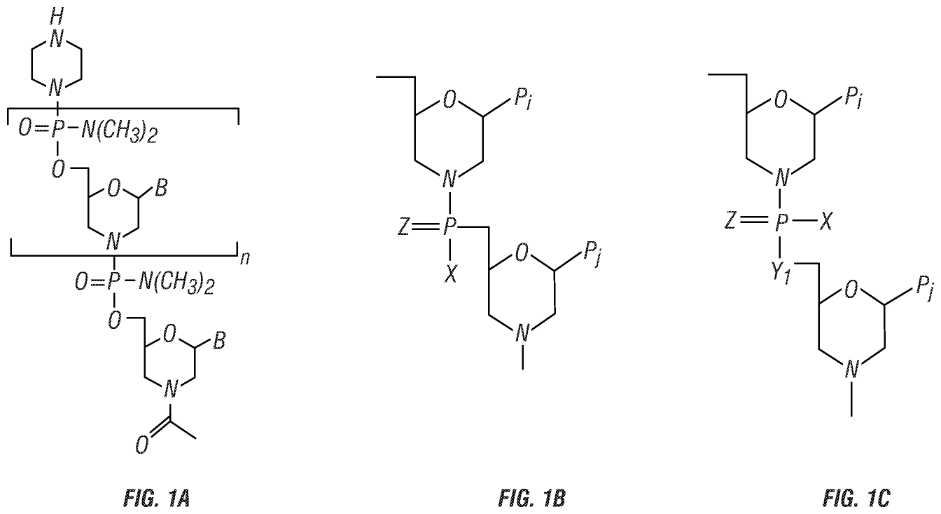

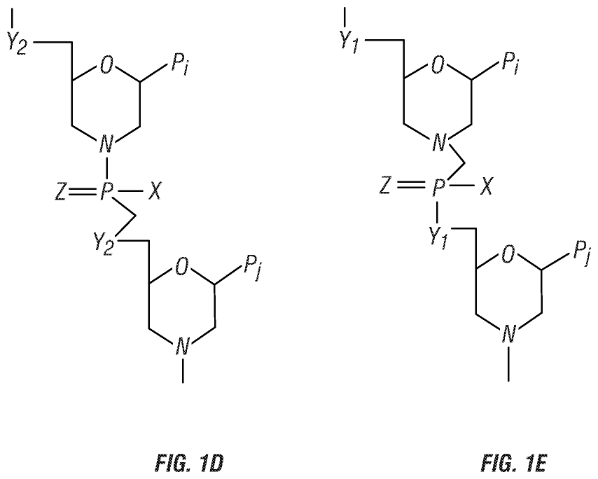

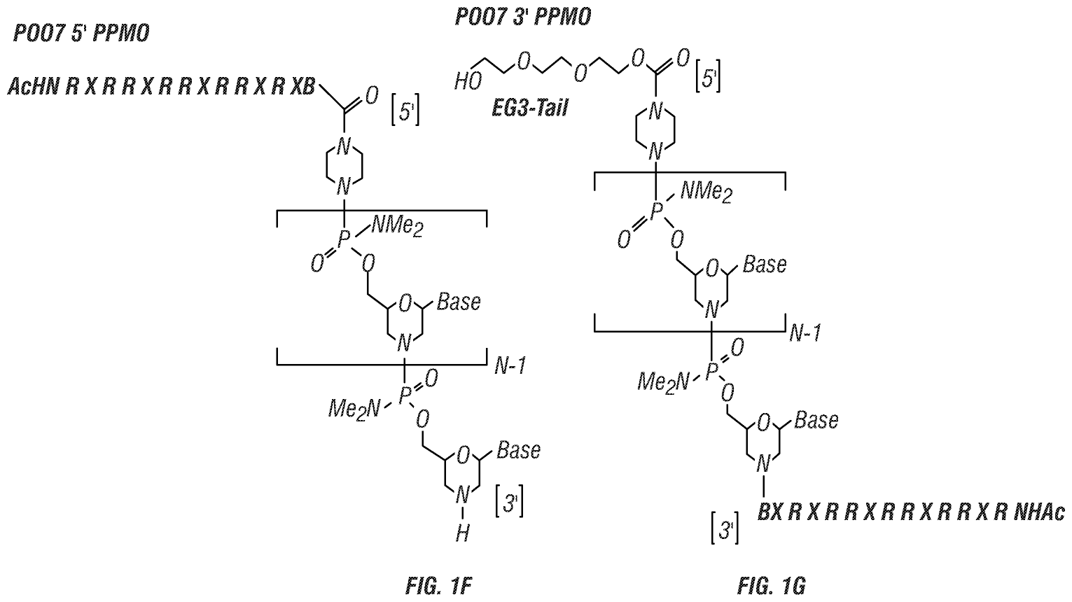

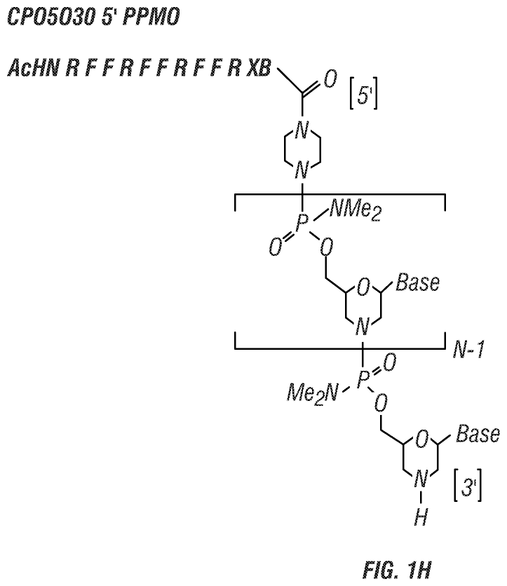

Also included are antisense oligomer that comprise a sequence of nucleotides of the formula in FIGS. 1A-1E. In FIGS. 1A-B is a purine or pyrimidine base-pairing moiety effective to bind, by base-specific hydrogen bonding, to a base in a polynucleotide. Y₁ or Y₂ may be oxygen, sulfur, nitrogen, or carbon, preferably oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy include 1-6 carbon atoms. The Z moieties may be sulfur or oxygen, and are preferably oxygen.

Accordingly, various embodiments of the disclosure include a substantially uncharged antisense morpholino oligomer, composed of morpholino subunits and phosphorus-containing intersubunit linkages joining a morpholino nitrogen of one subunit to a 5′-exocyclic carbon of an adjacent subunit, and having (a) about 10-40 nucleotide bases, and (b) a targeting sequence of sufficient length and complementarity to specifically hybridize to a bacterial mRNA target sequence that encodes a protein associated with a biochemical pathway and/or cellular process, as described herein. In some instances, the oligomer is conjugated to a cell-penetrating peptide (CPP).

In various aspects, an antisense oligomer of the disclosure includes a compound of formula (I):

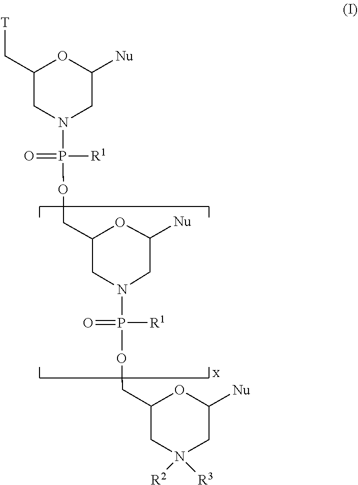

or a pharmaceutically acceptable salt thereof,

where each Nu is a nucleobase which taken together forms a targeting sequence;

X is an integer from 9 to 38;

T is selected from OH and a moiety of the formula:

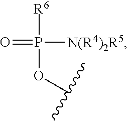

where each R⁴ is independently C₁-C₆ alkyl, and R⁵ is selected from an electron pair and H, and R⁶ is selected from OH, —N(R′)CH₂C(O)NH₂, and a moiety of the formula:

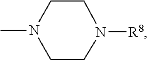

where:
R⁷ is selected from H and C₁-C₆ alkyl, and
R⁸ is selected from G, —C(O)—R⁹OH, acyl, trityl, and 4-methoxytrityl, where:
   R⁹ is of the formula —(O-alkyl)_{y}- wherein y is an integer from 3 to 10 and each of
   the y alkyl groups is independently selected from C₂-C₆ alkyl;
each instance of R¹ is —N(R¹⁰)₂R¹¹ wherein each R¹⁰ is independently C₁-C₆ alkyl, and R¹¹ is
selected from an electron pair and H;
R² is selected from H, G, acyl, trityl, 4-methoxytrityl, benzoyl, stearoyl, and a moiety of the formula:

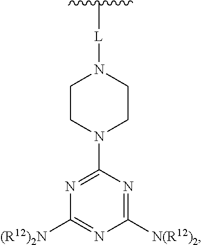

where L is selected from —C(O)(CH₂)₆C(O)— and —C(O)(CH₂)₂S₂(CH₂)₂C(O)—, and each R¹² is of the formula —(CH₂)₂OC(O)N(R¹⁴)₂ wherein each R¹⁴ is of the formula —(CH₂)₆NHC(═NH)NH₂; and

R³ is selected from an electron pair, H, and C₁-C₆ alkyl,
wherein G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP, and —C(O)CH₂NH—CPP, or G is of the formula:

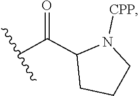

wherein the CPP is attached to the linker moiety by an amide bond at the CPP carboxy terminus, with the proviso that only one instance of G is present,

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

In some embodiments, X is from 9 to 18. In certain embodiments, X is 9, 10, 11, 12, 13, 14, 15, 16, 17, 18, 19, 20, 21, 22, 23, 24, 25, 26, 27, 28, 29 or 30.

In certain embodiments, T is selected from:

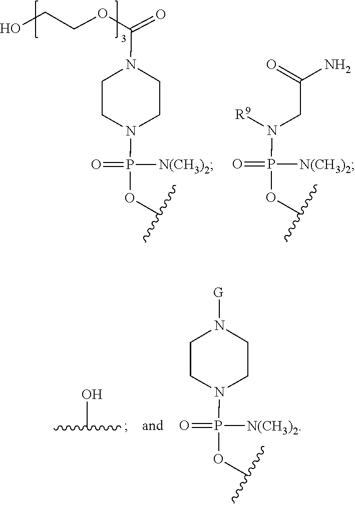

In some embodiments, R² is selected from H, G, acyl, trityl, 4-methoxytrityl, benzoyl, and stearoyl.

In various embodiments, T is selected from:

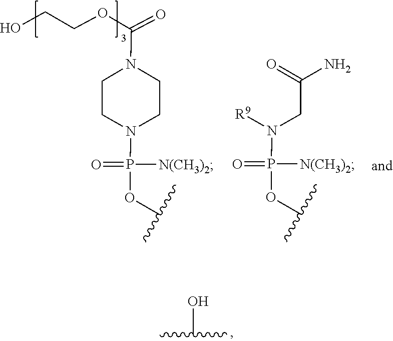

### and R² is G.

In some embodiments, T is of the formula:

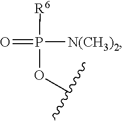

R⁶ is of the formula:

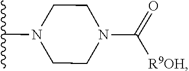

### and R² is G.

In certain embodiments, T is of the formula:

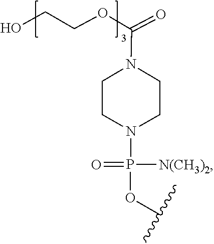

### and R² is G.

In certain embodiments, T is of the formula:

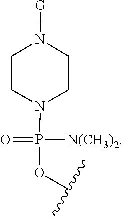

In some embodiments, R² is G or T is of the formula:

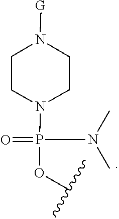

In some embodiments, R² is selected from H, acyl, trityl, 4-methoxytrityl, benzoyl, and stearoyl.

In various embodiments, R² is selected from H or G, and R³ is selected from an electron pair or H. In a particular embodiment, R² is G. In some embodiments, R² is H or acyl. In some embodiments, each R¹ is —N(CH₃)₂. In some embodiments, at least one instance of R¹ is —N(CH₃)₂. In certain embodiments, each instance of R¹ is —N(CH₃)₂.

In various embodiments of the disclosure, an antisense oligomer of the disclosure includes a compound of formula (II):

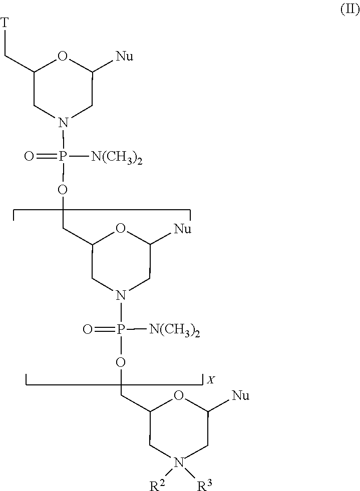

or a pharmaceutically acceptable salt thereof,

where each Nu is a nucleobase which taken together forms a targeting sequence;

X is an integer from 9 to 28;

T is selected from:

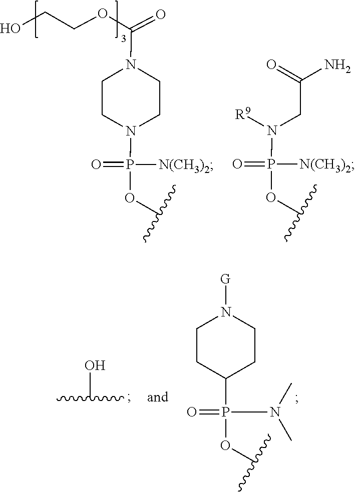

R² is selected from H, G, acyl, trityl, 4-methoxytrityl, benzoyl, and stearoyl; and

R³ is selected from an electron pair, H, and C₁-C₆ alkyl,

wherein G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,

and —C(O)CH₂NH—CPP, or G is of the formula:

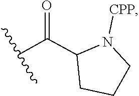

wherein the CPP is attached to the linker moiety by an amide bond at the CPP carboxy terminus, with the proviso that only one instance of G is present, and

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

In some embodiments, T is TEG as defined above, R² is G, and R³ is an electron pair or H. In certain embodiments, R² is selected from H, acyl, trityl, 4-methoxytrityl, benzoyl, and stearoyl and T is of the formula:

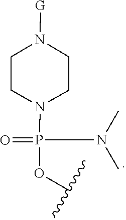

In some embodiments, R² is G or T is of the formula:

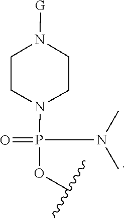

In various aspects, an antisense oligomer of the disclosure includes a compound of formula (III):

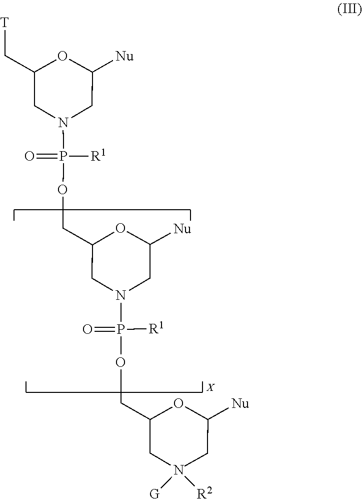

or a pharmaceutically acceptable salt thereof,

where each Nu is a nucleobase which taken together forms a targeting sequence;

X is an integer from 9 to 28;

T is selected from:

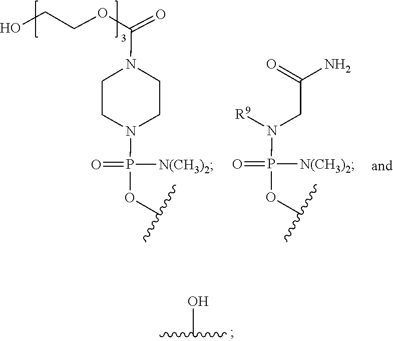

each instance of R¹ is —N(R¹⁰)₂R¹¹ wherein each R¹⁰ is independently C₁-C₆ alkyl, and R¹¹ is selected from an electron pair and H;

R² is selected from an electron pair, H, and C₁-C₆ alkyl; and

G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,

and —C(O)CH₂NH—CPP, or G is of the formula:

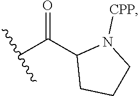

wherein the CPP is attached to the linker moiety by an amide bond at the CPP carboxy terminus,

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

In some embodiments, at least one instance of R¹ is —N(CH₃)₂. In certain embodiments, each instance of R¹ is —N(CH₃)₂.

In various aspects, an antisense oligomer of the disclosure includes a compound of formula (IV):

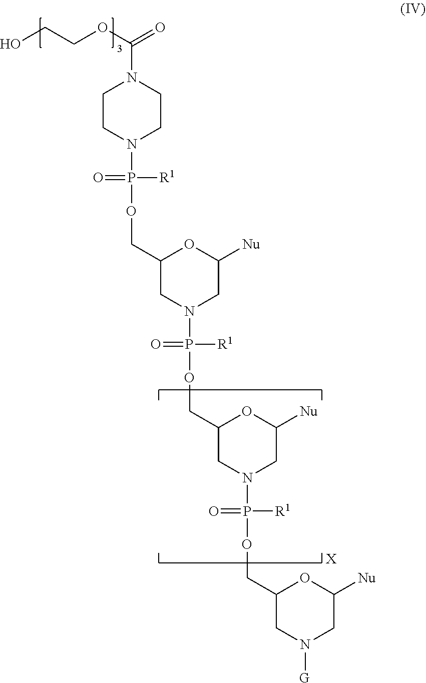

or a pharmaceutically acceptable salt thereof, wherein:

X is an integer from 9 to 28;

each Nu is a nucleobase which taken together forms a targeting sequence;

each instance of R¹ is —N(R¹⁰)₂R¹¹ wherein each R¹⁰ is independently C₁-C₆ alkyl, and R¹¹ is selected from an electron pair and H; and

G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,

and —C(O)CH₂NH—CPP, or G is of the formula:

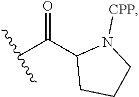

wherein the CPP is attached to the linker moiety by an amide bond at the CPP carboxy terminus. In some embodiments, at least one instance of R¹ is —N(CH₃)₂. In certain embodiments, each instance of R¹ is —N(CH₃)₂,

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

In various aspects, an antisense oligomer of the disclosure can be a compound of formula (V):

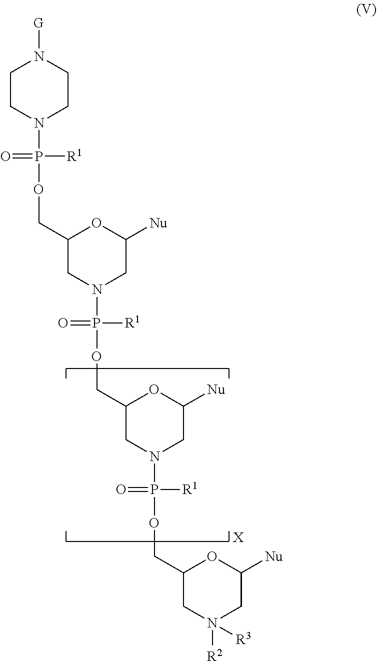

wherein:

X is an integer from 9 to 18;

each Nu is a nucleobase which taken together forms a targeting sequence;

each instance of R¹ is —N(R¹⁰)₂R¹¹ wherein each R¹⁰ is independently C₁-C₆ alkyl, and R¹¹ is selected from an electron pair and H;

R² is selected from H, trityl, 4-methoxytrityl, acyl, benzoyl, and stearoyl; and

R³ is selected from an electron pair, H, and C₁-C₆ alkyl,

wherein G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,

and —C(O)CH₂NH—CPP, or G is of the formula:

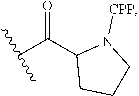

wherein the CPP is attached to the linker moiety by an amide bond at the CPP carboxy terminus. In some embodiments, at least one instance of R¹ is —N(CH₃)₂. In certain embodiments, each instance of R¹ is —N(CH₃)₂,

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

In various aspects, an antisense oligomer of the disclosure includes a compound of formula (VI):

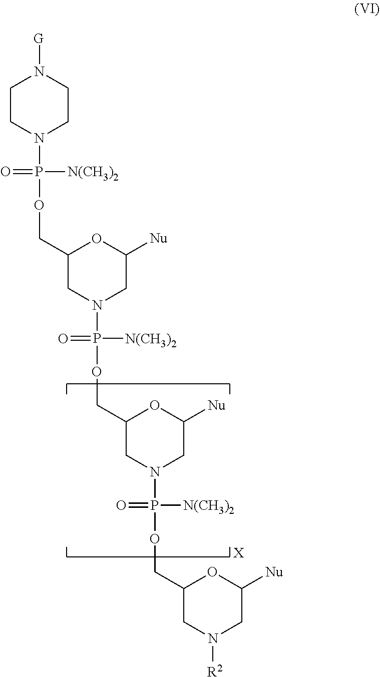

or a pharmaceutically acceptable salt thereof, wherein:

X is an integer from 9 to 28;

each Nu is a nucleobase which taken together forms a targeting sequence;

R² is selected from H or acyl; and

G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,

and —C(O)CH₂NH—CPP, or G is of the formula:

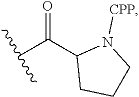

wherein the CPP is attached to the linker moiety by an amide bond at the CPP carboxy terminus,

wherein the targeting sequence specifically hybridizes to a bacterial mRNA target sequence that encodes a protein associated with a biochemical pathway and/or cellular process.

In some embodiments, the bacterium is from the genera Klebsiella. In some embodiments, the bacterium is an antibiotic-resistant strain of Klebsiella. In some embodiments, the bacterium is a multi-drug resistant (MDR) strain of Klebsiella. In some embodiments, the bacterium is Klebsiella pneumonia, for example, selected from Table 3.

In various embodiments, the targeting sequence specifically hybridizes to a Klebsiella pneumonia mRNA target sequence that encodes a protein associated with a biochemical pathway and/or cellular process.

The antisense oligomers can be prepared by stepwise solid-phase synthesis, employing methods known in the art and described in the references cited herein.

B. Cell-Penetrating Peptides

In certain embodiments, the antisense oligomer is conjugated to a cell-penetrating peptide (CPP). In some embodiments, the CPP is an arginine-rich peptide. By “arginine-rich carrier peptide” is meant that the CPP has at least 2, and preferably 2, 3, 4, 5, 6, 7, or 8 arginine residues, each optionally separated by one or more uncharged, hydrophobic residues, and optionally containing about 6-14 amino acid residues. FIGS. 1F-1H show exemplary chemical structures of CPP-PMO conjugates used in the Examples, including 5′ and 3′ PMO conjugates. Exemplary CPPs are provided in Table C1 (SEQ ID NOS: 13-27).

TABLE C1
Exemplary Cell-Penetrating Peptides

| Name | Sequence | SEQ ID NO: |
|---|---|---|
| (RXR)₄ | RXRRXRRXRRXR | 13 |
| (RFF)₃R | RFFRFFRFFR | 14 |
| (RXR)₄XB | RXRRXRRXRRXRXB | 15 |
| (RFF)₃RXB | RFFRFFRFFRXB | 16 |
| (RFR)₄ | RFRRFRRFRRFR | 17 |
| (RYR)₄ | RYRRYRRYRRYR | 18 |
| (RGR)₄ | RGRRGRRGRRGR | 19 |
| (RFR)₄XB | RFRRFRRFRRFRXB | 20 |
| (RYR)₄XB | RYRRYRRYRRYRXB | 21 |
| (RGR)₄XB | RGRRGRRGRRGRXB | 22 |
| (RFF)₃RXB | RFFRFFRFFRXB | 23 |
| (RFF)₃RG | RFFRFFRFFRG | 24 |
| (R)₆G | RRRRRRG | 25 |
| (RXR)₄G | RXRRXRRXRRXRG | 26 |
| (R)₆ | RRRRRR | 27 |

X is 6-aminohexanoic acid; B is β-alanine; F is phenylalanine; Y is tyrosine; G is glycine; R is arginine

In some embodiments, the CPP is linked at its C-terminus to the 3′-end or the 5′-end of the oligomer via a 1, 2, 3, 4, or 5 amino acid linker.

CPPs, their synthesis, and methods of conjugating a CPP to an oligomer are detailed, for example, in International Patent Application Publication Nos. WO 2004/097017, WO 2009/005793, and WO 2012/150960, which are all incorporated by reference in their entirety.

In some embodiments, the CPP is linked at its C-terminus to the 3′-end or the 5′-end of the oligomer via a 1, 2, 3, 4, or 5 amino acid linker. In particular embodiments, including antisense oligomer compounds of formula (I)-(VI), the linkers can include: —C(O)(CH₂)₅NH—CPP (X linker), —C(O)(CH₂)₂NH—CPP (B linker), —C(O)(CH₂)₂NHC(O)(CH₂)₅NH—CPP (XB peptide linker), and —C(O)CH₂NH—CPP (G linker), or formula:

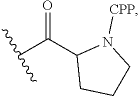

wherein the CPP is attached to the linker moiety by an amide bond at the CPP carboxy terminus. In some embodiments of the disclosure, including antisense oligomer compounds of formula (I)-(VI), G is selected from SEQ ID NOS: 15-16 and 20-26. In various embodiments, including antisense oligomer compounds of formula (I)-(VI), the CPP is selected from SEQ ID NO: 13-14, 17-19, and 27.

In some embodiments, including antisense oligomer compounds of formula (I)-(VI), the CPP is selected from:

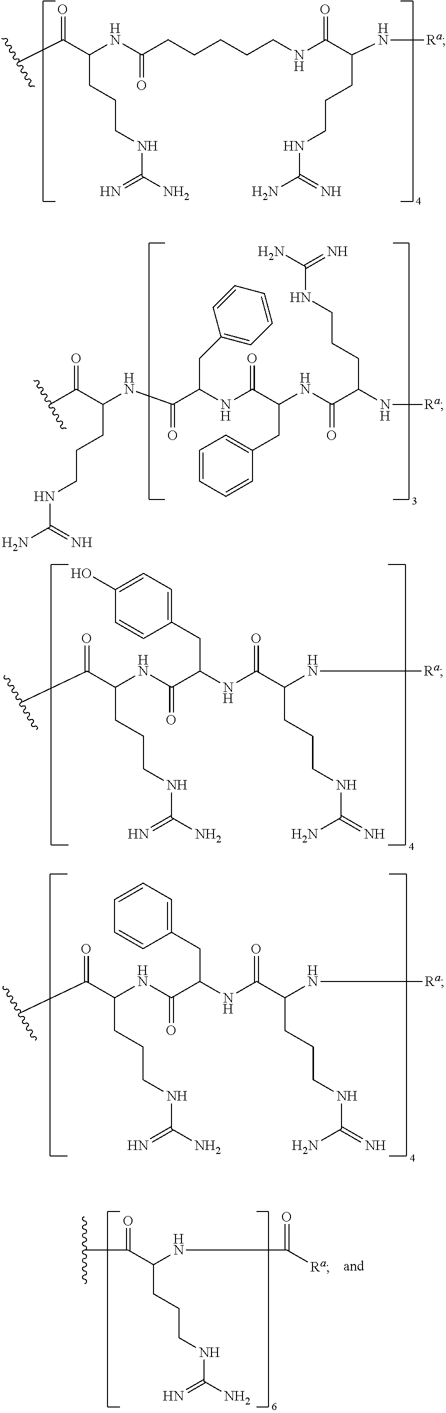

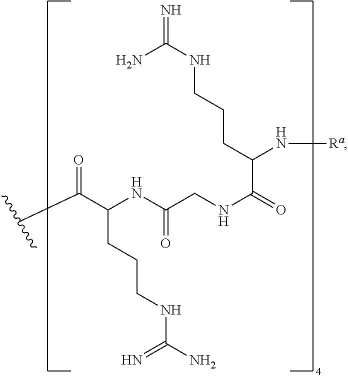

wherein R^{a} is selected from H, acetyl, benzoyl, and stearoyl.

In some embodiments, including antisense oligomer compounds of formula (I)-(VI), G is selected from:

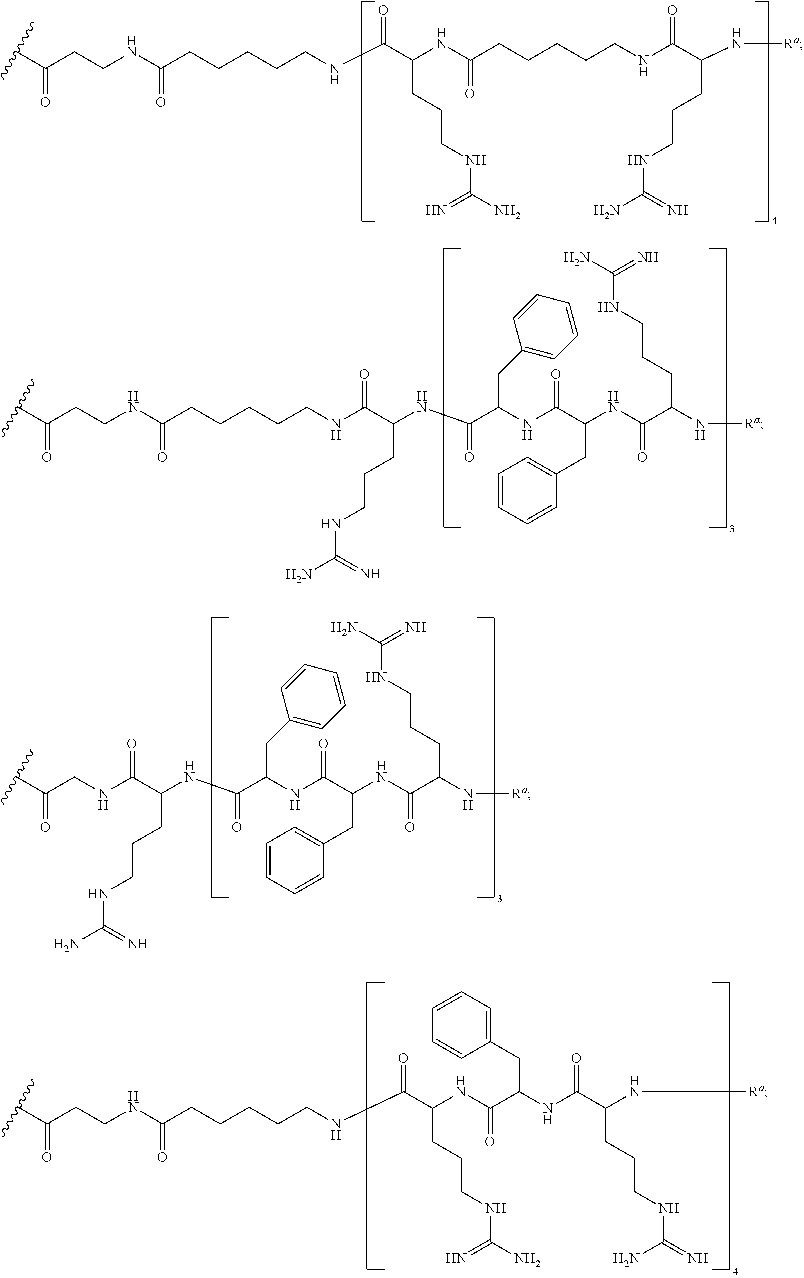

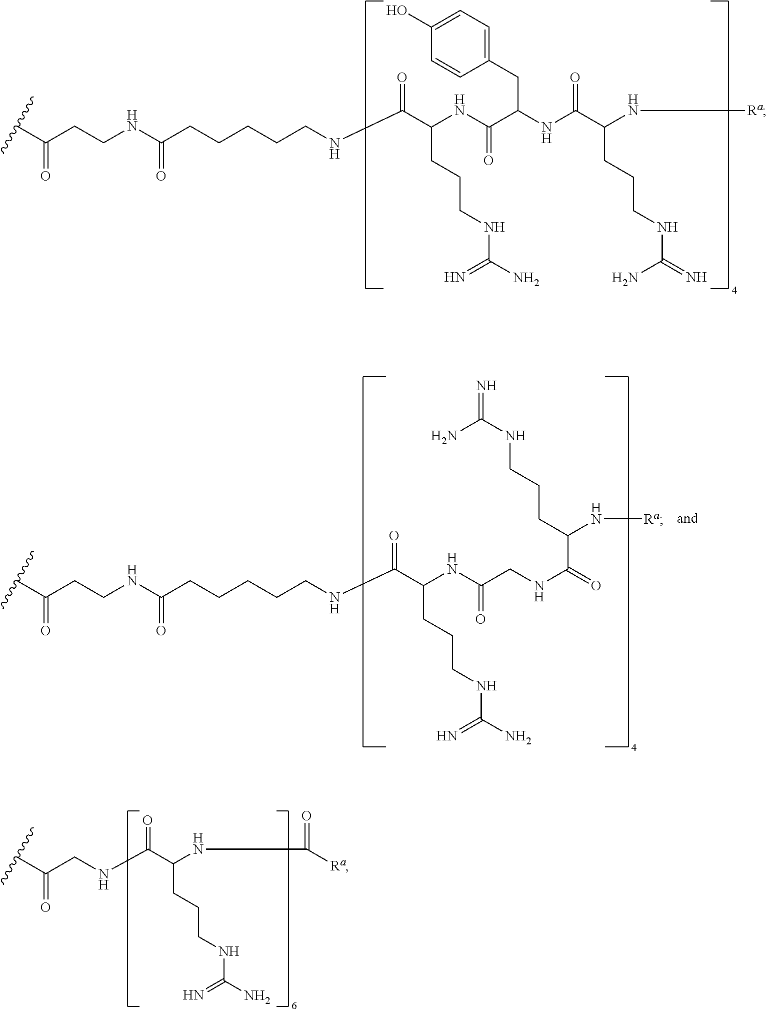

wherein R^{a} is selected from H, acetyl, benzoyl, and stearoyl.

In various aspects, an antisense oligomer of the disclosure, or a pharmaceutically acceptable salt thereof, includes an antisense oligomer of the formula (VII) selected from:

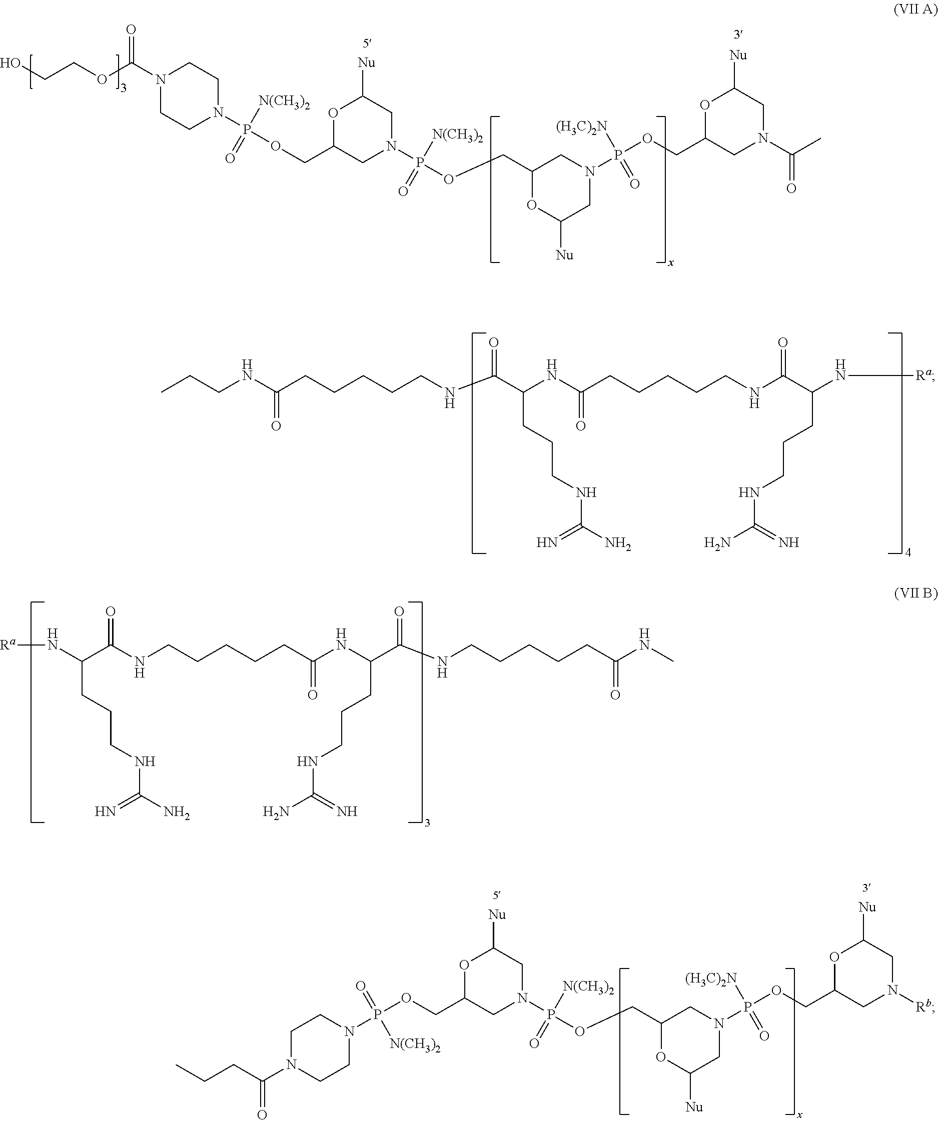

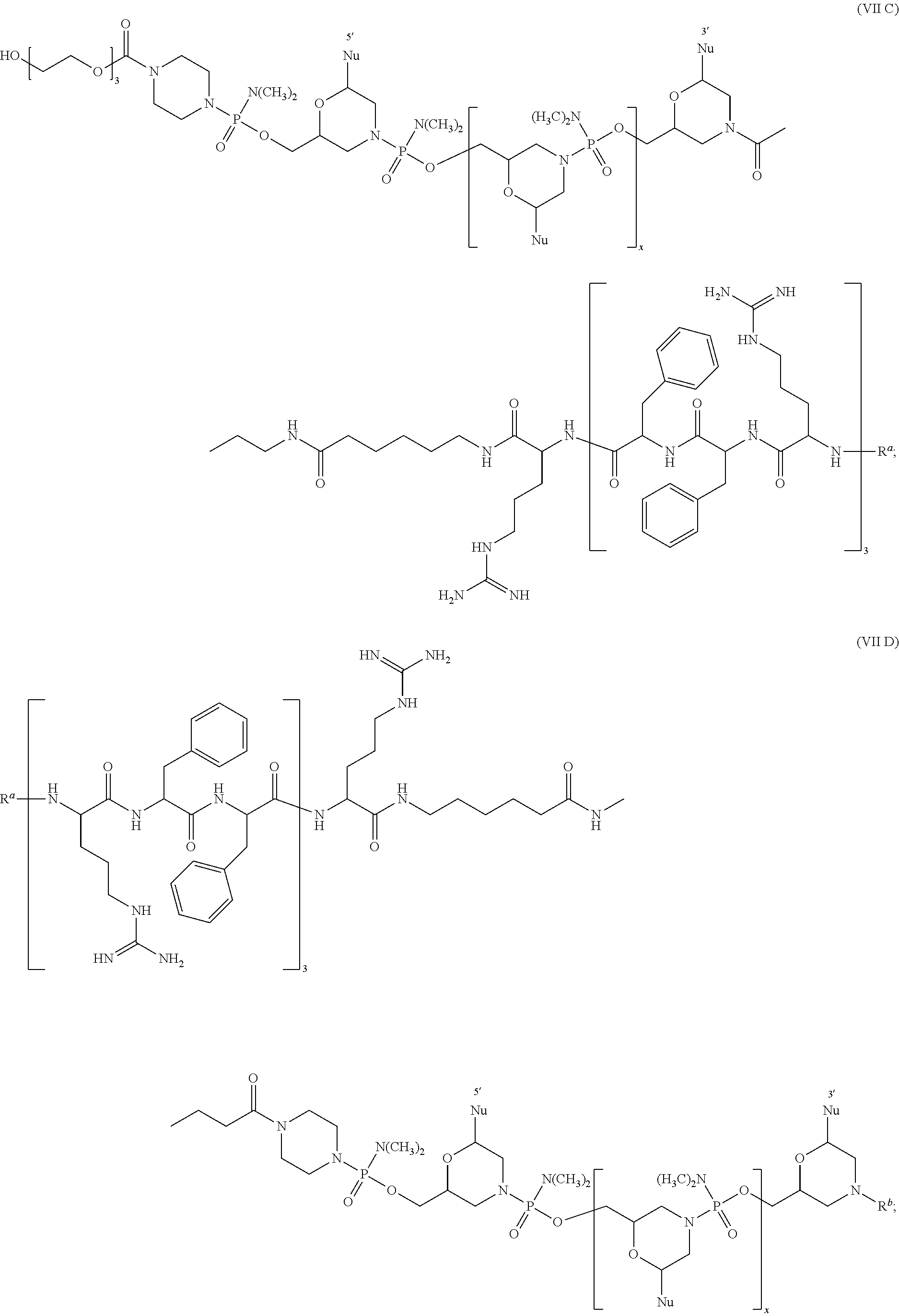

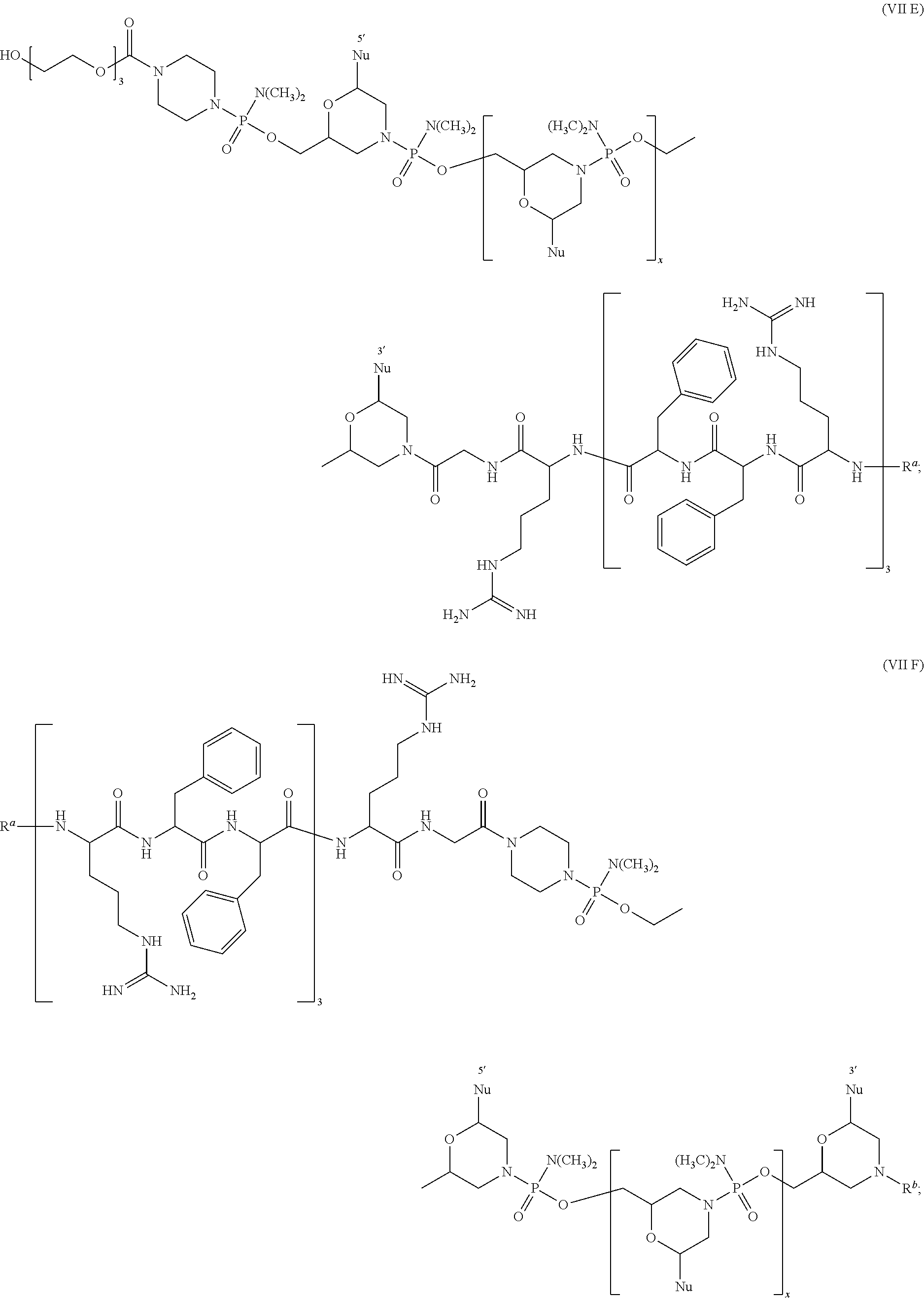

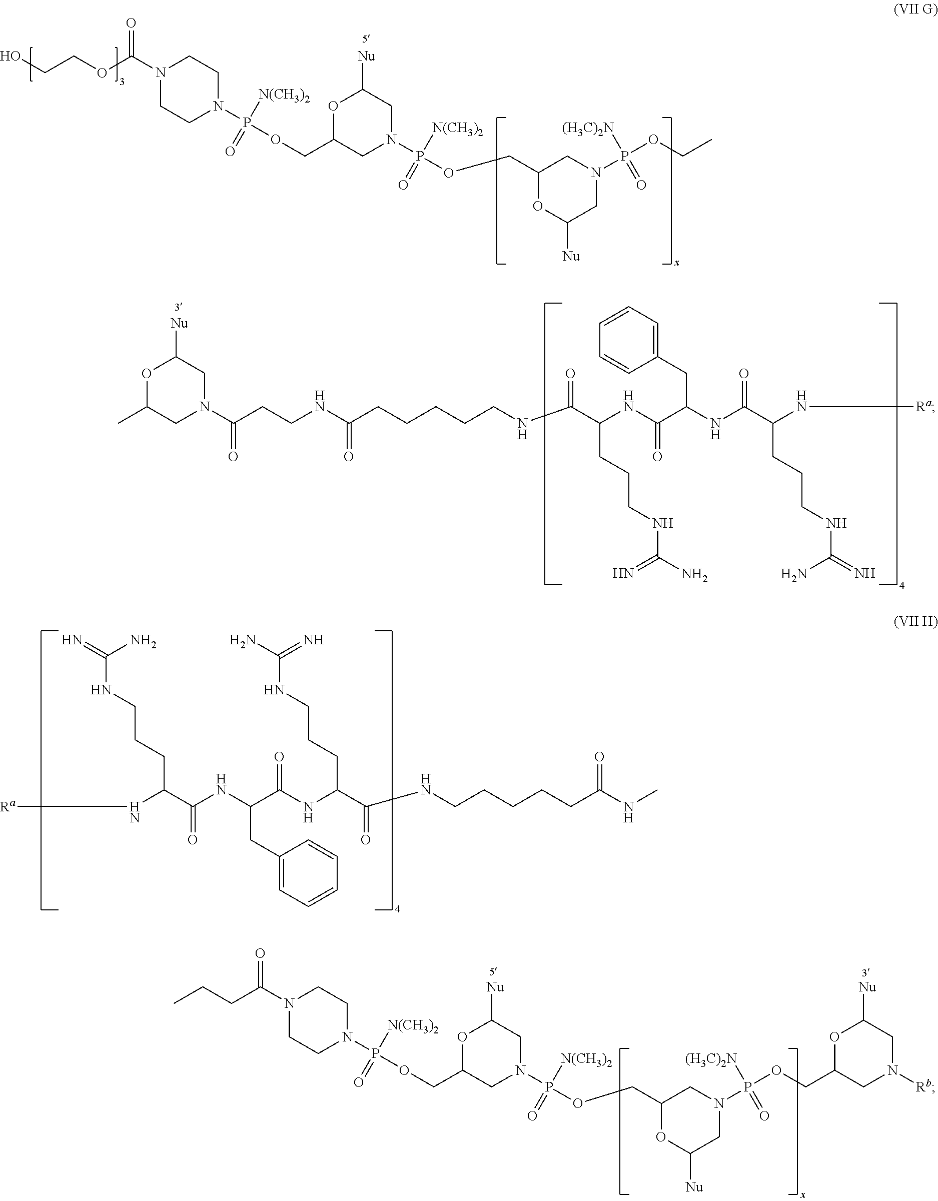

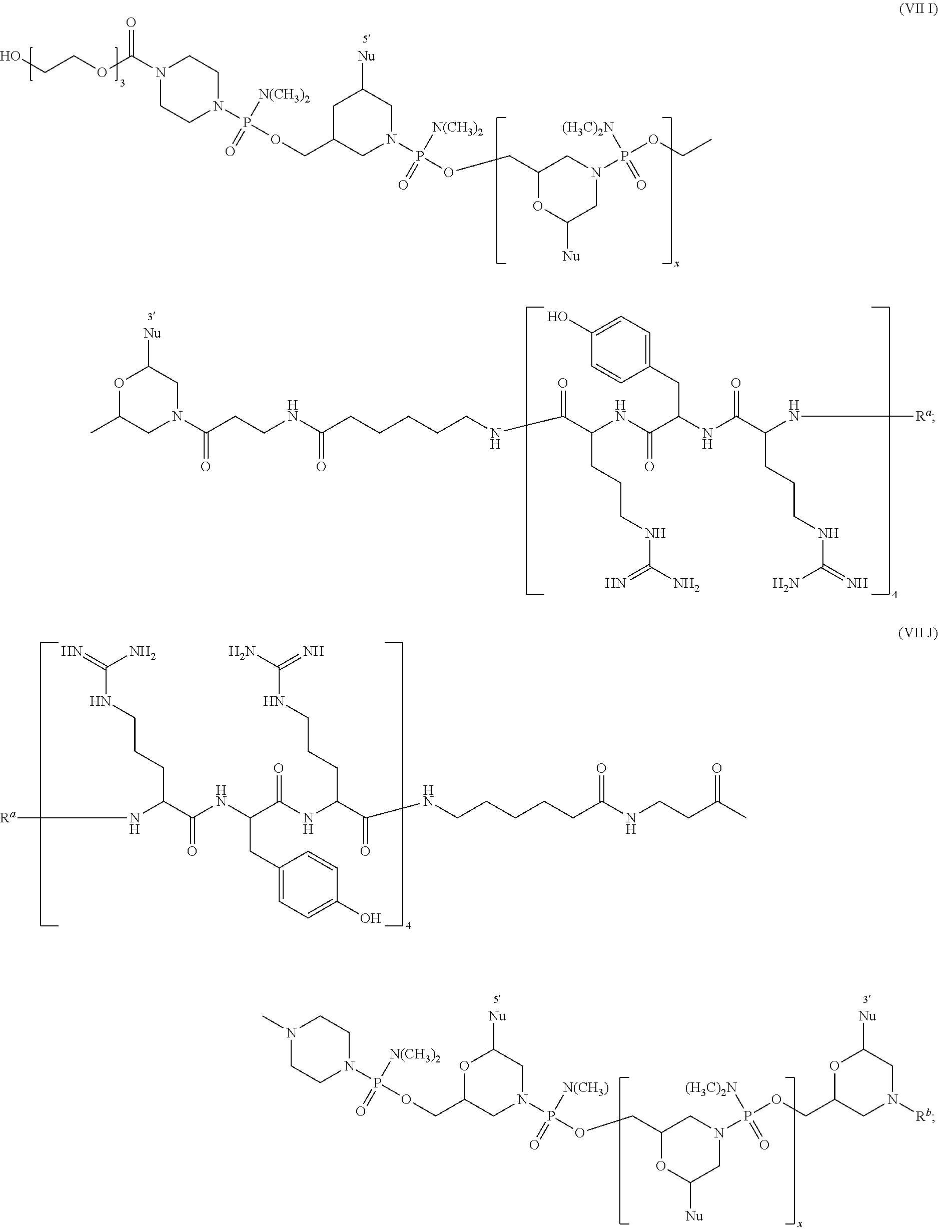

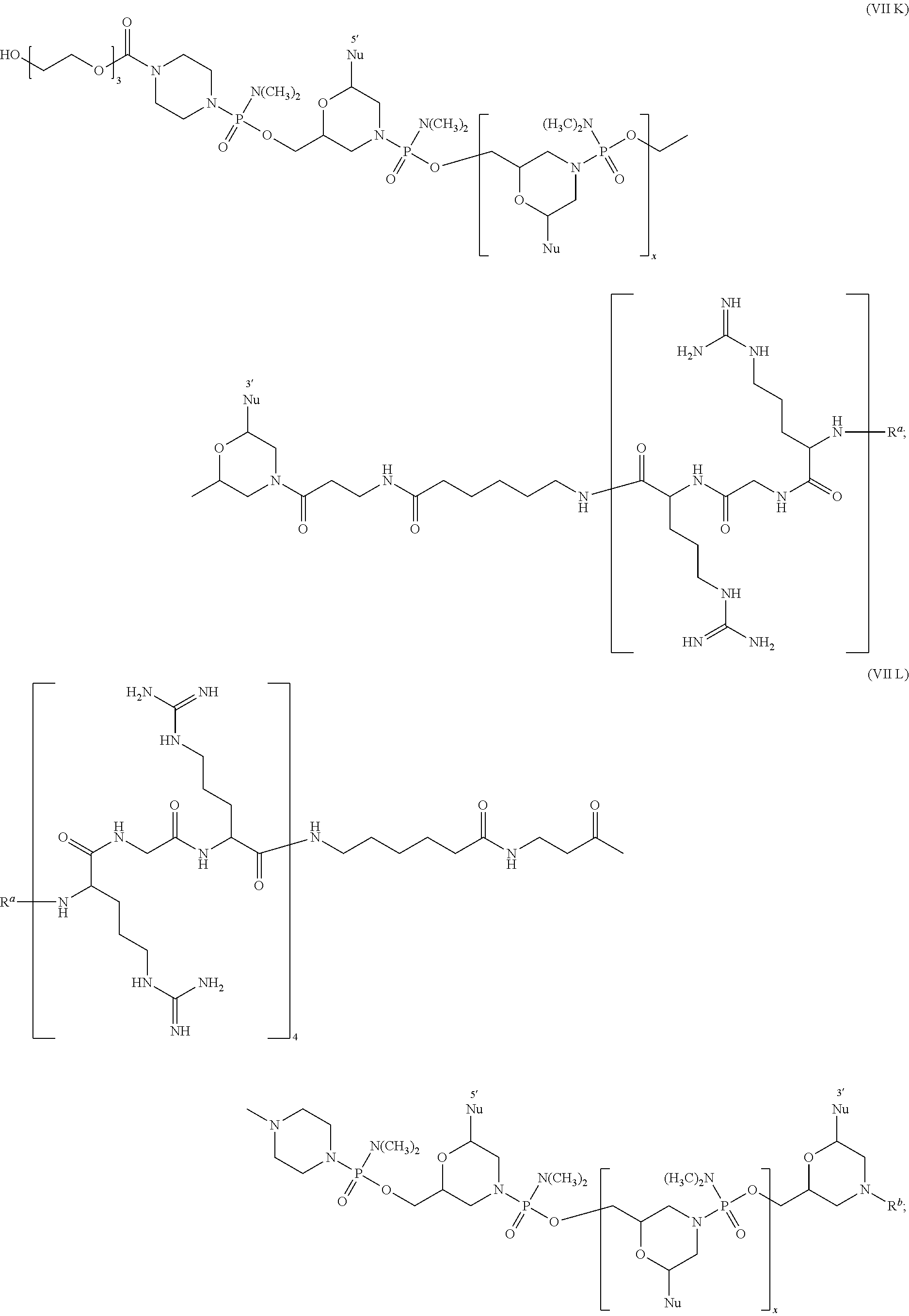

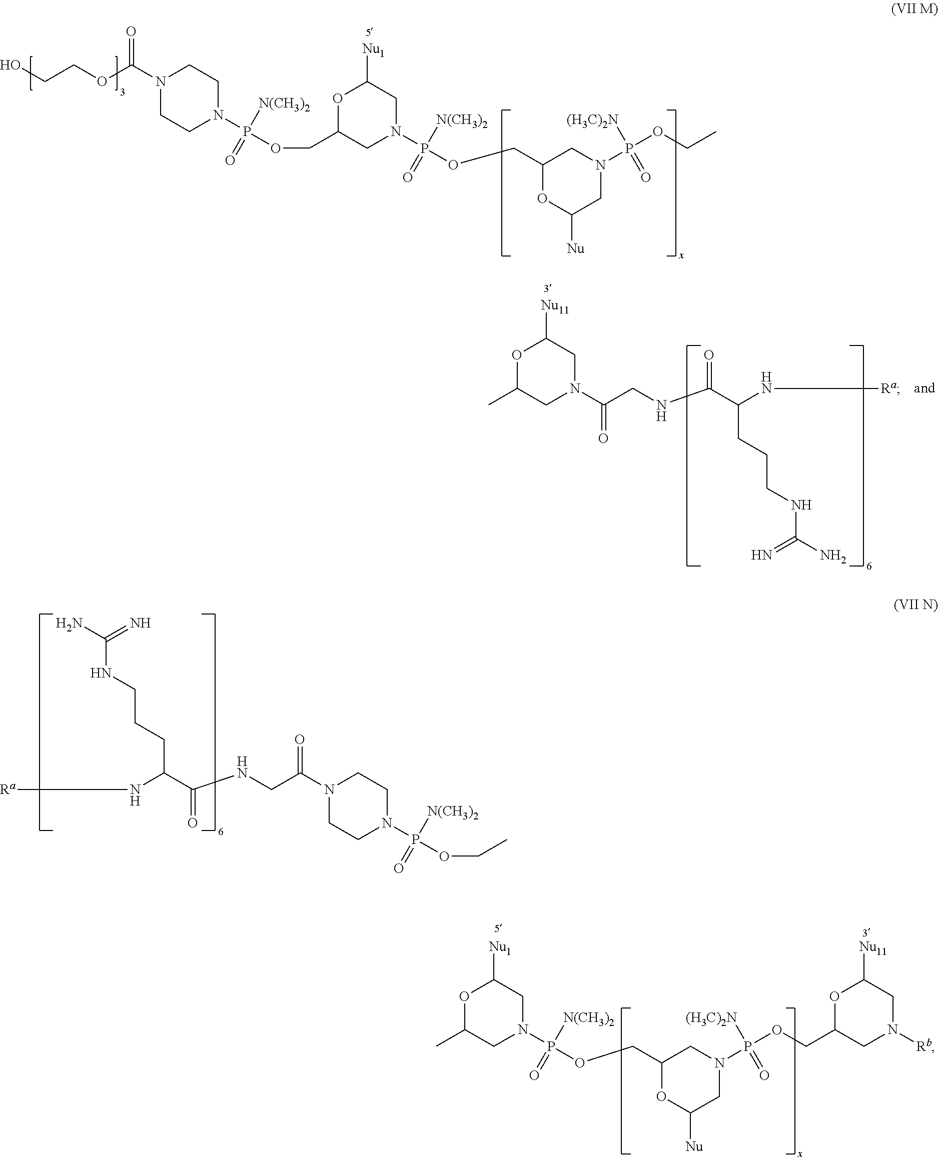

wherein X is an integer from 9 to 38, R^{a} is selected from H, acetyl, benzoyl, and stearoyl, R^{b} is selected from H, acetyl, benzoyl, stearoyl, trityl, and 4-methoxytrityl, and each Nu is a purine or pyrimidine base-pairing moiety which taken together form a targeting sequence described above.

C. Antisense Oligomer Targeting Sequences

In various embodiments of the antisense oligomers of the disclosure, including the antisense oligomer compounds of formulas (I)-(VII), the targeting sequence can specifically hybridizes to a bacterial mRNA target sequence that encodes a protein associated with a biochemical pathway and/or cellular process. In some embodiments, the target sequence comprises a translational start codon of the bacterial mRNA and/or a sequence within about 30, 20, or 10 or so bases upstream or downstream of the translational start codon of the bacterial mRNA.

In various embodiments, the protein associated with a biochemical pathway and/or cellular process may be a fatty acid biosynthesis protein. In some embodiments, the fatty acid biosynthesis protein can be an acyl carrier protein. In certain embodiments, the acyl carrier protein may be AcpP. In some embodiments, the fatty acid biosynthesis protein may be an acyl carrier protein synthase. In certain embodiments, the acyl carrier protein synthase may be FabB. In some embodiments, the target sequence may be SEQ ID NOs:1-3, wherein thymine bases (T) are optionally uracil bases (U). In certain embodiments, the targeting sequence comprises or consists of at least one of the targeting sequences in Table 1A (e.g., SEQ ID NOS:1-3), comprises or consists of a fragment of at least 10 contiguous nucleotides of a targeting sequence in Table 1A (e.g., SEQ ID NOS:1-3), or comprises or consists of a variant having at least 80% sequence identity to a targeting sequence in Table 1A (e.g., SEQ ID NOS: 1-3), wherein thymine bases (T) are optionally uracil bases (U).

In some embodiments, the protein associated with a biochemical pathway and/or cellular process may be a peptidoglycan biosynthesis protein. In certain embodiments, the peptidoglycan biosynthesis protein can be a UDP-N-acetylglucosamine 1-carboxyvinyltransferase. In some embodiments, the UDP-N-acetylglucosamine 1-carboxyvinyltransferase may be MurA.

In some embodiments, the protein associated with a biochemical pathway and/or cellular process is a ribosomal protein. In certain embodiments, the ribosomal protein is a 50S ribosomal protein L28. In some embodiments, the 50S ribosomal protein L28 is RpmB. In certain embodiments, the ribosomal protein is a 30S ribosomal protein. In some embodiments, the 30S ribosomal protein is RpsJ.

In some embodiments, the protein associated with a biochemical pathway and/or cellular process is a DNA or chromosomal replication protein. In certain embodiments, the DNA or chromosomal replication protein is a helicase. In some embodiments, the helicase is DnaB.

In certain embodiments, the protein associated with a biochemical pathway and/or cellular process is a lipopolysaccharide biosynthesis protein. In some embodiments, the lipopolysaccharide biosynthesis protein is a N-acetylglucosamine deacetylase. In some embodiments, the N-acetylglucosamine deacetylase is LpxC.

In some embodiments, the protein associated with a biochemical pathway and/or cellular process is a cell division protein. In certain embodiments, the cell division protein is a protein that assembles into a ring at the future site of the septum of bacterial cell division. For example, in some embodiments, the protein that assembles into a ring at the future site of the septum of bacterial cell division is FtsZ.

In some embodiments where the protein associated with a biochemical pathway and/or cellular process may be a murein biosynthesis protein, cell division protein, global gene regulatory protein, fatty acid biosynthesis protein, ribosomal protein, DNA/chromosomal replication protein, transcription protein, translation initiation protein, lipopolysaccharide biosynthesis protein, nucleic acid biosynthesis protein, intermediary metabolism protein, RNA biosynthesis protein, protein biosynthesis protein, and peptidoglycan biosynthesis protein, or other protein described herein, the targeting sequence comprises or consists of at least one of the targeting sequences set forth in Table 1B (e.g., SEQ ID NOS: 4-11), comprises or consists of a fragment of at least 10 contiguous nucleotides of a targeting sequence in Table 1B (e.g., SEQ ID NOS: 4-11), or comprises or consists of a variant having at least 80% sequence identity to a targeting sequence in Table 1B (e.g., SEQ ID NOS: 4-11), wherein thymine bases (T) are optionally uracil bases (U).

In certain embodiments, including the antisense oligomer compounds of formulas (I)-(VII), the targeting sequence is selected from:

a)
SEQ ID NO: 1
(TGCTCATACTC);
b)
SEQ ID NO: 2
(CTTCGATAGTG);
c)
SEQ ID NO: 3
(CGTTTCATTAA);

wherein X is 9, wherein thymine bases (T) may be uracil bases (U).

In some embodiments, including the antisense oligomer compounds of formulas (I)-(VII), the targeting sequence is selected from:

a)
SEQ ID NO: 4
(GTCTATTCTCC);
b)
SEQ ID NO: 5
(GACATGTCTAT);
c)
SEQ ID NO: 6
(TGGTTCTGCAT);
d)
SEQ ID NO: 7
(TTTATCCATTG);
e)
SEQ ID NO: 8
(GTTCAAACATA);
f)
SEQ ID NO: 9
(AGTTTCTCTCC);
g)
SEQ ID NO: 10
(TTCCTGCCATA);
h)
SEQ ID NO: 11
(TTTGATCATCG);

wherein X is 9, and wherein thymine bases (T) may be uracil bases (U).

D. Exemplary Antisense Oligomers

Exemplary antisense oligomers (AONs) of the disclosure include those described in Tables 2A-B below. Exemplary scramble control AONs are provided in Table 2C.

TABLE 2A
Exemplary Fatty Acid Biosynthesis-Associated Targeting Sequences AONs

| | | | TS | | | |
|---|---|---|---|---|---|---|
| | | | SEQ | 5′ | 3′ | CPP |
| PPMO | Target | Targeting | ID | Attachment | Attachment | SEQ |
| Name | Gene | Sequence (TS)* | NO: | *** | ** | ID NO. |
| 0016 | acpP | TGCTCATACTC | 1 | R₆G | acetyl | 25 |
| 0276 | acpP | TGCTCATACTC | 1 | (RXR)₄XB | acetyl | 15 |
| 0398 | acpP | TGCTCATACTC | 1 | TEG | (RXR)₄XB | 15 |
| 0399 | acpP | TGCTCATACTC | 1 | TEG | R₆G | 25 |
| 0076 | acpP | CTTCGATAGT | 2 | (RXR)₄XB | acetyl | 15 |
| | | G | | | | |
| 0099 | acpP | CTTCGATAGT | 2 | TEG | (RYR)₄XB | 21 |
| | | G | | | | |
| 0141 | acpP | CTTCGATAGT | 2 | TEG | (RGR)₄XB | 22 |
| | | G | | | | |
| 0155 | acpP | CTTCGATAGT | 2 | TEG | R₆G | 25 |
| | | G | | | | |
| 0205 | acpP | CTTCGATAGT | 2 | TEG | (RFR)₄XB | 20 |
| | | G | | | | |
| 0605 | acpP | CTTCGATAGT | 2 | (RYR)₄XB | acetyl | 21 |
| | | G | | | | |
| 0606 | acpP | CTTCGATAGT | 2 | (RGR)₄XB | acetyl | 22 |
| | | G | | | | |
| 0621 | acpP | CTTCGATAGT | 2 | (RFR)₄XB | acetyl | 20 |
| | | G | | | | |
| 0802 | acpP | CTTCGATAGT | 2 | R₆G | acetyl | 25 |
| | | G | | | | |
| 0948 | acpP | CTTCGATAGT | 2 | TEG | (RXR)₄XB | 15 |
| | | G | | | | |
| 0279 | fabB | CGTTTCATTAA | 3 | (RXR)₄XB | acetyl | 15 |

TABLE 2B
Exemplary AONS targeting other biochemical pathways and/or cellular
processes

| | | | TS | | | |
|---|---|---|---|---|---|---|
| | | | SEQ | 5′ | 3′ | CPP |
| PPMO | Target | Targeting | ID | Attachment | Attachment | SEQ |
| Name | Gene | Sequence (TS)* | NO: | *** | ** | ID NO. |
| 0017 | rpmB | GTCTATTCTCC | 4 | R₆G | acetyl | 25 |
| 0283 | rpmB | GTCTATTCTCC | 4 | (RXR)₄XB | acetyl | 15 |
| 0282 | rpmB | GACATGTCTA | 5 | (RXR)₄XB | acetyl | 15 |
| | | T | | | | |
| 0277 | rpsJ | TGGTTCTGCAT | 6 | (RXR)₄XB | acetyl | 15 |
| 0278 | murA | TTTATCCATTG | 7 | (RXR)₄XB | acetyl | 15 |
| 0280 | ftsZ | GTTCAAACAT | 8 | (RXR)₄XB | acetyl | 15 |
| | | A | | | | |
| 0281 | ftsZ | AGTTTCTCTCC | 9 | (RXR)₄XB | acetyl | 15 |
| 0284 | dnaB | TTCCTGCCATA | 10 | (RXR)₄XB | acetyl | 15 |
| 0285 | lpxC | TTTGATCATCG | 11 | (RXR)₄XB | acetyl | 15 |

TABLE 2C
Exemplary Scramble Control AONs

| | | | TS | | | |
|---|---|---|---|---|---|---|
| | | | SEQ | 5′ | 3′ | CPP |
| PPMO | Target | Targeting | ID | Attachment | Attachment | SEQ |
| Name | Gene | Sequence (TS)* | NO: | *** | ** | ID NO. |
| 1073 | scramble | TCTCAGATGG | 12 | (RXR)₄AB | acetyl | 15 |
| | | T | | | | |
| 1344 | scramble | TCTCAGATGG | 12 | R₆G | acetyl | 25 |
| | | T | | | | |

### IV. Methods of Use and Formulations

Embodiments of the present disclosure include methods of using the antisense oligomers described herein to reduce the expression and activity of one or more bacterial proteins associated with biochemical pathways and/or cellular processes. Certain embodiments include methods of using the antisense oligomers to reduce replication, proliferation, or growth of a bacteria, for example, to treat a bacterial infection in a subject, either alone or in combination with one or more additional antimicrobial agents. In some instances, the antisense oligomers increase the susceptibility of the bacterium to one or more antimicrobial agents.

Also included are pharmaceutical compositions comprising the antisense oligomers, typically in combination with a pharmaceutically-acceptable carrier. Certain pharmaceutical compositions can further comprise one or more antimicrobial agents. The methods provided herein can be practiced in vitro or in vivo.

For example, certain embodiments include methods of treating a bacterial infection in a subject, comprising administering to a subject in need thereof (e.g., subject having or at risk for having a bacterial infection) an antisense oligomer or pharmaceutical composition described herein. Also included are methods of reducing replication of a bacteria, comprising contacting the bacterium with an antisense oligomer described herein.

In some embodiments, the bacterium is from the genus Klebsiella.

Klebsiella is a genus of a Gram-negative, nonmotile, encapsulated, lactose-fermenting, facultative anaerobic, rod-shaped bacterium that includes the species Klebsiella pneumoniae, which is responsible for the vast majority of Klebsiella-related pathogenesis. Thus, in some embodiments, the bacterium is any members of the genera Klebsiella. In specific embodiments, the bacterium is Klebsiella pneumoniae. In some embodiments, the bacterium is selected from one or more of the strains in Table 3.

TABLE 3
Strains of K. pneumoniae

BAA 2146
NDM1-A
NDM1-B
NDM1-C
NDM1-D
Hm 748
Hm 749
Hm 750
Hm 751
Pneu3426
Pneu3427
Pneu3190
Pneu3290
NR 15410
NR 15411
NR 15412
NR 15416
NR 15417
OR-001
OR-002
OR-003
OR-004
OR-005
OR-006
OR-007
OR-008
OR-009
OR-010
OR-011
OR-012
OR-013
OR-014
OR-015
OR-016
OR-017
OR-018
OR-019
OR-020

In certain embodiments, the bacterium is a multi-drug resistance (MDR) strain of bacteria. Multiple drug resistance (MDR), multi-drug resistance or multiresistance is a condition enabling disease-causing microorganisms (bacteria, viruses, fungi or parasites) to resist distinct antimicrobials such as antibiotics, antifungal drugs, antiviral medications, antiparasitic drugs, and others. In particular embodiments, the bacterium is extensively-drug resistant (XDR) or pan-drug resistant (PDR). In some embodiments, the bacterium is an extended-spectrum β-lactamase (ESBLs) producing Gram-negative bacteria, or a multi-drug-resistant gram negative rod (MDR GNR) MDRGN bacteria. In specific embodiments, the bacterium is MDR Klebsiella, for example, MDR Klebsiella pneumoniae.

Examples of genes associated with biochemical pathways and/or cellular processes include fatty acid biosynthesis genes (and their related proteins) such as acpP and/or fab genes, for example, fabB. In particular embodiments, the bacterium comprises or expresses the acpP gene, which encodes an acyl carrier protein. In some embodiments, the bacterium comprises or expresses the fabB gene, which encodes an carrier protein synthase. In some embodiments, the bacterium that comprises or expresses one or more genes associated with fatty acid biosynthesis (e.g., acpP, fabB) is a Klebsiella species, for example, Klebsiella pneumoniae.

Examples of genes associated with biochemical pathways and/or cellular processes include peptidoglycan biosynthesis genes (and their related proteins). In particular embodiments, the bacterium comprises or expresses the murA gene, which encodes a UDP-N-acetylglucosamine 1-carboxyvinyltransferase. In some embodiments, the bacterium that comprises or expresses one or more peptidoglycan biosynthesis genes (e.g., murA) is a Klebsiella species, for example, Klebsiella pneumoniae.

Examples of genes associated with biochemical pathways and/or cellular processes include ribosomal protein genes (and their related proteins). In particular embodiments, the bacterium comprises or expresses the rpmB gene, which encodes a 50S ribosomal protein L28. In particular embodiments, the bacterium comprises or expresses the rpsJ gene, which encodes a 30S ribosomal protein. In some embodiments, the bacterium that comprises or expresses one or more ribosomal protein genes (e.g., rpmB, rpsJ) is a Klebsiella species, for example, Klebsiella pneumoniae.

Examples of genes associated with biochemical pathways and/or cellular processes include cell division genes (and their related proteins). In particular embodiments, the bacterium comprises or expresses the ftsZ gene, which encodes a protein that assembles into a ring at the future site of the septum of bacterial cell division. In specific embodiments, the bacterium that comprises or expresses one or more genes associated with cell division (e.g., ftsZ) is a Klebsiella species, for example, Klebsiella pneumoniae.

Examples of genes associated with DNA or chromosomal replication include topoisomerases and helicases (and their related proteins). In some embodiments, the bacterium comprises or expresses the dnaB gene, which encodes a helicase. In specific embodiments, the bacterium that comprises or expresses one or more genes associated with DNA or chromosomal replication (e.g., dnaB) is a Klebsiella species, for example, Klebsiella pneumoniae.

Examples of genes associated with lipopolysaccharide biosynthesis include deacetylases such as N-acetylglucosamine deacetylase. In particular embodiments, the bacterium comprises or expresses the lpxC gene, which encodes an N-acetylglucosamine deacetylase. In specific embodiments, the bacterium that comprises or expresses one or more genes associated with lipopolysaccharide biosynthesis (e.g., lpxC) is a Klebsiella species, for example, Klebsiella pneumoniae.

In some embodiments, the antisense oligomer reduces expression of the gene(s) associated with biochemical pathways and/or cellular processes in the bacteria or bacterium. For instance, in some embodiments, the antisense oligomer reduces expression by about or at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to a control (e.g., absence of the antisense oligomer, scrambled oligomer, prior to contacting with the oligomer), or by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to a control. In some embodiments, the antisense oligomer reduces expression of one or more of AcpP, FabB, MurA, RpmB, RpsJ, DnaB, FtsZ, and/or LpxC and the bacterium is a Klebsiella species which comprises or expresses one or more of AcpP, FabB, MurA, RpmB, RpsJ, DnaB, FtsZ, and/or LpxC. Gene or protein expression can be measured in vitro (see, e.g., the Examples) or in vivo.

In some embodiments, the antisense oligomer reduces or inhibits the growth of the bacteria or bacterium. For instance, in some embodiments, the antisense oligomer reduces growth of the bacteria or bacterium by about or at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to a control (e.g., absence of the antisense oligomer, scrambled oligomer, prior to contacting with the oligomer), or by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to a control. Bacterial growth can be measured in vitro (see, e.g., the Examples) or in vivo. In particular embodiments, the antisense oligomer that reduces growth of the bacterium is targeted against expression a protein associated with a biochemical pathway and/or cellular process selected from one or more of AcpP, FabB, MurA, RpmB, RpsJ, DnaB, FtsZ, and/or LpxC and the bacterium is a Klebsiella species which comprises or expresses one or more of AcpP, FabB, MurA, RpmB, RpsJ, DnaB, FtsZ, and/or LpxC. In some embodiments, as described herein, the antisense oligomer is employed in combination with one or more antimicrobial agents, for example, to reduce (e.g., synergistically reduce) the growth of the bacteria or bacterium.

In some embodiments, the methods are practiced in vivo, and comprise administering the antisense oligomer to a subject in need thereof, for example, a subject in need thereof that is infected or at risk for being infected by one or more of the bacteria described herein. In certain embodiments, the subject in need thereof is immunocompromised. In some embodiments, the subject has underlying lung disease, such as pneumonia, cystic fibrosis (CF), and/or chronic granulomatous disease (CGD).

In some embodiments, the bacterium is in the lung(s) of the subject, for example, as a bacterial lung infection. In some embodiments, administration of the antisense oligomer reduces bacterial lung burden or bacterial lung counts by about or at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to a control (e.g., absence of the antisense oligomer, scrambled oligomer, prior to contacting with the oligomer), or by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to a control.

In certain embodiments, the bacterium has formed or is at risk for forming a biofilm in the subject. In some instances, the biofilm forms or is at risk for forming at the surfaces of one or more tissues (for example, lung tissues), and/or biomaterials such as medical implants and catheters. In certain embodiments, administration of the antisense oligomer reduces biofilm formation or existing biofilm by about or at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to a control (e.g., absence of the antisense oligomer, scrambled oligomer, prior to contacting with the oligomer), or by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to a control.

The antisense oligomers described herein can thus be administered to subjects to treat (prophylactically or therapeutically) an infection by any of the bacteria described herein. In conjunction with such treatment, pharmacogenomics (e.g., the study of the relationship between an individual's genotype/phenotype and that individual's response to a foreign compound or drug) may be considered. Differences in metabolism of therapeutics can lead to severe toxicity or therapeutic failure by altering the relation between dose and blood concentration of the pharmacologically active drug.

Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Effective delivery of the antisense oligomer to the target nucleic acid is one aspect of treatment. Routes of antisense oligomer delivery include, but are not limited to, various systemic routes, including oral and parenteral routes, e.g., intravenous, subcutaneous, intraperitoneal, and intramuscular, as well as inhalation, transdermal, and topical delivery. The appropriate route may be determined by one of skill in the art, as appropriate to the condition of the subject under treatment. Vascular or extravascular circulation, the blood or lymph system, and the cerebrospinal fluid are some non-limiting sites where the antisense oligomers may be introduced. Direct CNS delivery may be employed, for instance, intracerebral, intraventricular, or intrathecal administration may be used as routes of administration.

In certain embodiments, the antisense oligomers can be delivered by transdermal methods (e.g., via incorporation of the antisense oligomers into, e.g., emulsions, with such antisense oligomers optionally packaged into liposomes). Such transdermal and emulsion/liposome-mediated methods of delivery are described for delivery of antisense oligomers in the art, e.g., in U.S. Pat. No. 6,965,025, the contents of which are incorporated in their entirety by reference herein.

The antisense oligomers described herein may also be delivered via an implantable device. Design of such a device is an art-recognized process, with, e.g., synthetic implant design described in, e.g., U.S. Pat. No. 6,969,400, the contents of which are incorporated by reference.

Antisense oligomers can be introduced into cells using art-recognized techniques (e.g., transfection, electroporation, fusion, liposomes, colloidal polymeric particles and viral and non-viral vectors as well as other means known in the art). The method of delivery selected will depend at least on the oligomer chemistry, the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligomers may be delivered using, e.g., methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (see, e. g., Dokka and Rojanasakul, Advanced Drug Delivery Reviews 44:35-49, incorporated by reference in its entirety).

The antisense oligomers may be administered in any convenient vehicle or carrier which is physiologically and/or pharmaceutically acceptable. Such a composition may include any of a variety of standard pharmaceutically acceptable carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration. “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions

The compounds (e.g., antisense oligomers, antimicrobial agents) described herein may generally be utilized as the free acid or free base. Alternatively, the compounds described herein may be used in the form of acid or base addition salts. Acid addition salts of the free amino compounds described herein may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids.

Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” is intended to encompass any and all acceptable salt forms.

In addition, prodrugs are also included within the context of this disclosure. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this disclosure wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the antisense oligomers described herein. Further, in the case of a carboxylic acid (—COOH), esters may be employed, such as methyl esters, ethyl esters, and the like.

In some instances, liposomes may be employed to facilitate uptake of the antisense oligomer into cells (see, e.g., Williams, S. A., Leukemia 10(12):1980-1989, 1996; Lappalainen et al., Antiviral Res. 23:119, 1994; Uhlmann et al., antisense oligomers: a new therapeutic principle, Chemical Reviews, Volume 90, No. 4, 25 pages 544-584, 1990; Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligomers may be administered in microspheres or microparticles. (See, e.g., Wu, G. Y. and Wu, C. H., J. Biol. Chem. 262:4429-4432, 30 1987). Alternatively, the use of gas-filled microbubbles complexed with the antisense oligomers can enhance delivery to target tissues, as described in U.S. Pat. No. 6,245,747. Sustained release compositions may also be used. These may include semipermeable polymeric matrices in the form of shaped articles such as films or microcapsules.

In certain embodiments, the antisense oligomer is administered to a mammalian subject, e.g., human or domestic animal, exhibiting the symptoms of a bacterial infection (e.g., antibiotic resistance or MDR bacterial infection), in a suitable pharmaceutical carrier. In some aspects, the subject is a human subject, e.g., a patient diagnosed as having a bacterial infection. In particular embodiments, the antisense oligomer is contained in a pharmaceutically acceptable carrier, and is delivered orally. In some embodiments, the antisense oligomer is contained in a pharmaceutically acceptable carrier, and is delivered intravenously (i.v.).

In some embodiments, the antisense oligomer is administered in an amount and manner effective to result in a peak blood concentration of at least 200-400 nM antisense oligomer. Typically, one or more doses of antisense oligomer are administered, generally at regular intervals, for a period of about one to two weeks. Certain doses for oral administration are from about 1-1000 mg oligomer per 70 kg. In some cases, doses of greater than 1000 mg oligomer/patient may be necessary. For i.v. administration, some doses are from about 0.5 mg to 1000 mg oligomer per 70 kg. The antisense oligomer may be administered at regular intervals for a short time period, e.g., daily for two weeks or less. However, in some cases the antisense oligomer is administered intermittently over a longer period of time. Administration may be followed by, or concurrent with, administration of an antimicrobial (e.g., antibiotic) or other therapeutic treatment, as described herein. The treatment regimen may be adjusted (dose, frequency, route, etc.) as indicated, based on the results of immunoassays, other biochemical tests and physiological examination of the subject under treatment.

An effective in vivo treatment regimen using the antisense oligomers described herein may vary according to the duration, dose, frequency and route of administration, as well as the condition of the subject under treatment (i.e., prophylactic administration versus administration in response to localized or systemic infection). Accordingly, such in vivo therapy will often include monitoring by tests appropriate to the particular type of disorder or bacterial infection under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligomer described herein may be determined from biological samples (tissue, blood, urine etc.) taken from a subject prior to, during and subsequent to administration of the antisense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

### V. Combination Therapies

Certain embodiments include combination therapies, for example, the administration of antisense oligomers in combination with antimicrobial agents such as antibiotics. Combination therapies can be employed, for example, to increase the sensitivity or susceptibility of a given bacteria to one or more antimicrobial agents, and thereby improve the therapeutic outcome (e.g., resolution of the infection). Likewise, certain combination therapies can be employed, for example, to reduce or reverse the resistance of a given bacteria to one or more antimicrobial agents. In particular embodiments, the antisense oligomer reduces the minimum inhibitory concentration (MIC) of an antibiotic against a given bacterium. In certain embodiments, the antisense oligomer and the antimicrobial agent display synergy in reducing bacterial growth and/or increasing bacterial cell-killing. Also included are pharmaceutical compositions, as described herein, which comprise an antisense oligomer and an antimicrobial agent such as antibiotic.

In some embodiments, the antisense oligomer and the antimicrobial agent are administered separately. In certain embodiments, the antisense oligomer and the antimicrobial agent are administered sequentially. In some embodiments, the antisense oligomer and the antimicrobial agent are administered concurrently, for example, as part of the same or different pharmaceutical composition.

Examples of antimicrobial agents (e.g., antibiotics) that can be administered in combination with an antisense oligomer include beta-lactam antibiotics such as carbapenems, penicillin and penicillin derivatives (or penams), ampicillin, chloramphenicol, cephalosporins (e.g., Cefacetrile (cephacetrile), Cefadroxil (cefadroxyl; Duricef), Cephalexin (cefalexin; Keflex), Cefaloglycin (cephaloglycin), Cefalonium (cephalonium), Cefaloridine (cephaloradine), Cefalotin (cephalothin; Keflin), Cefapirin (cephapirin; Cefadryl), Cefatrizine, Cefazaflur, Cefazedone, Cefazolin (cephazolin; Ancef, Kefzol), Cefradine (cephradine; Velosef), Cefroxadine, Ceftezole, Cefaclor (Ceclor, Distaclor, Keflor, Raniclor), Cefonicid (Monocid), Cefprozil (cefproxil; Cefzil), Cefuroxime (Zefu, Zinnat, Zinacef, Ceftin, Biofuroksym, Xorimax), Cefuzonam, Cefmetazole, Cefotetan, Cefoxitin, loracarbef (Lorabid); Cephamycins: cefbuperazone, cefmetazole (Zefazone), cefminox, cefotetan (Cefotan), cefoxitin (Mefoxin), Cefotiam (Pansporin), Cefcapene, Cefdaloxime, Cefdinir (Sefdin, Zinir, Omnicef, Kefnir), Cefditoren, Cefetamet, Cefixime (Fixx, Zifi, Suprax), Cefmenoxime, Cefodizime, Cefotaxime (Claforan), Cefovecin (Convenia), Cefpimizole, Cefpodoxime (Vantin, PECEF), Cefteram, Ceftibuten (Cedax), Ceftiofur, Ceftiolene, Ceftizoxime (Cefizox), Ceftriaxone (Rocephin), Cefoperazone (Cefobid), Ceftazidime (Meezat, Fortum, Fortaz), latamoxef (moxalactam), Cefclidine, cefepime (Maxipime), cefluprenam, cefoselis, Cefozopran, Cefpirome (Cefrom), Cefquinome, flomoxef, Ceftobiprole, Ceftaroline, Cefaloram, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, Cefuracetime), and monobactams (e.g., aztreonam, tigemonam, nocardin A, tabtoxin); aminoglycosides such as tobramycin, gentamicin, kanamycin a, amikacin, dibekacin, sisomicin, netilmicin, neomycin B, neomycin C, neomycin E (paromomycin), and streptomycin; tetracyclines such as tetracycline, chlortetracycline, oxytetracycline, demeclocycline, lymecycline, meclocycline, methacycline, minocycline, rolitetracycline, and doxycyline; sulfonamides such as sulfacetamide, sulfadiazine, sulfadimidine, sulfafurazole, sulfisomidine, sulfadoxine, sulfamethoxazole, sulfamoxole, sulfadimethoxine, sulfamethoxypyridazine, sulfametoxydiazine, sulfadoxine, and sulfametopyrazine; quinolones such as cinoxacin, nalidixic acid, oxolinic acid (Uroxin), piromidic acid (Panacid), pipemidic acid (Dolcol) rosoxacin (Eradacil), ciprofloxacin (Alcipro, Ciprobay, Cipro, Ciproxin, ultracipro), enoxacin (Enroxil, Penetrex), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), balofloxacin (Baloxin), grepafloxacin (Raxar), levofloxacin (Cravit, Levaquin, Tavanic), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin) (Zymar—opth.), gemifloxacin (Factive), moxifloxacin (Acflox Woodward, Avelox, Vigamox, sitafloxacin (Gracevit), trovafloxacin (Trovan), prulifloxacin (Quisnon); oxazolidinones such as eperezolid, linezolid, posizolid, radezolid, ranbezolid, sutezolid, and tedizolid; polymyxins such as polysporin, neosporin, polymyxin B, polymyxin E (colistin); rifamycins such as rifampicin or rifampin, rifabutin, rifapentine, and rifaximin; lipiarmycins such as fidaxomicin; macrolides such as azithromycin, clarithromycin, dirithromycin, erythromycin, roxithromycin, telithromycin, carbomycin A, josamycin, kitasamycin, midecamycin/midecamycin acetate, oleandomycin, solithromycin, spiramycin, and troleandomycin; lincosamides such as lincomycin, clindamycin, and pirlimycin; cyclic lipopeptides such as daptomycin; glycopeptides such as vancomycin and teichoplanin; glycylcyclines such as tigecycline. Thus, any one or more of the foregoing antibiotics can be combined with any of the antisense oligomers described herein, for the treatment of any of the bacterium or bacteria described herein.

In some embodiments, the antimicrobial agent is selected from one or more of aminoglycoside antibiotics, tetracycline antibiotics, and β-lactam antibiotics, as described herein. In some of these and related embodiments, the bacterium comprises or expresses a gene selected from one or more of acpP and fabB, and the antisense oligomer is targeted against expression of the fatty acid biosynthesis gene. In some of these and related embodiments, the bacterium comprises or expresses a gene selected from one or more of murA, and the antisense oligomer is targeted against expression of the peptidoglycan biosynthesis gene. In some of these and related embodiments, the bacterium comprises or expresses a gene selected from one or more of rpmB and rpsJ, and the antisense oligomer is targeted against expression of the ribosomal protein gene. In some of these and related embodiments, the bacterium comprises or expresses a gene selected from one or more of ftsZ, and the antisense oligomer is targeted against expression of the cell division gene. In some of these and related embodiments, the bacterium comprises or expresses a gene selected from one or more of dnaB, and the antisense oligomer is targeted against expression of the DNA or chromosomal replication gene. In some of these and related embodiments, the bacterium comprises or expresses a gene selected from one or more of lpxC, and the antisense oligomer is targeted against expression of the lipopolysaccharide biosynthesis gene. In specific embodiments, the bacterium is Klebsiella pneumoniae, including MDR strains thereof.

In some embodiments, the antimicrobial agent is a beta-lactam antibiotic, as described herein. In particular embodiments, the antimicrobial agent is a carbapenem. Examples of carbapenems include meropenem, imipenem, ertapenem, doripenem, panipenem, biapenem, razupenem, tebipenem, lenapenem, tomopenem, and ampicillin. In specific embodiments, the antimicrobial agent is meropenem. In particular embodiments, the antimicrobial agent is a cephalosporin (cephem), penicillin or penicillin derivative (penam). In particular embodiments, the antisense oligomer reduces the MIC of a carbapenem such as meropenem against a bacteria, for example, a strain or MDR strain of Klebsiella pneumoniae. In some embodiments, the combination of the antisense oligomer and the carbapenem such as meropenem reduces (e.g., synergistically reduces) bacterial cell growth or increase (e.g., synergistically increases) bacterial cell-killing, for example, of a strain or MDR strain of Klebsiella pneumoniae.

In some embodiments, the antimicrobial agent is an aminoglycoside, as described herein. Examples of aminoglycosides include tobramycin, gentamicin, kanamycin a, amikacin, dibekacin, sisomicin, netilmicin, neomycin B, neomycin C, neomycin E (paromomycin), and streptomycin. In specific embodiments, the antimicrobial agent is tobramycin. In particular embodiments, the antisense oligomer reduces the MIC of an aminoglycoside such as tobramycin against a bacteria, for example, a strain or MDR strain of Klebsiella pneumoniae. In some embodiments, the combination of the antisense oligomer and the aminoglycoside such as tobramycin reduces (e.g., synergistically reduces) bacterial cell growth or increases (e.g., synergistically increases) bacterial cell-killing, for example, of a strain or MDR strain of Klebsiella pneumoniae.

In certain embodiments, the antimicrobial agent is a polymyxin such as colistin (polymyxin E), polysporin, neosporin, or polymyxin B. In specific embodiments, the antimicrobial agent is colistin. In particular embodiments, the antisense oligomer reduces the MIC of a polymyxin such as colistin against a bacteria, for example, a strain or MDR strain of Klebsiella pneumoniae. In some embodiments, the combination of the antisense oligomer and the polymyxin such as colistin reduces (e.g., synergistically reduces) bacterial cell growth or increases (e.g., synergistically increases) bacterial cell-killing, for example, of a strain or MDR strain of Klebsiella pneumoniae.

In certain embodiments, the antimicrobial agent includes one or more of ceftazidime, doxycycline, piperacillin, meropenem, chloramphenicol, and/or co-trimoxazole (trimethoprim/sulfamethoxazole).

In some embodiments, the antisense oligomer increases the susceptibility or sensitivity of a given bacterium to the antimicrobial agent, relative to the antimicrobial agent alone. For example, in certain embodiments, the antisense oligomer increases the susceptibility or sensitivity of the bacteria or bacterium to the antimicrobial agent by increasing the bactericidal (cell-killing) and/or bacteriostatic (growth-slowing) activity of the antimicrobial agent against the bacteria or bacterium being targeted, relative to the antimicrobial agent alone. In particular embodiments, the antisense oligomer increases the susceptibility or sensitivity by about or at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to the antimicrobial agent alone, or by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to the antimicrobial agent alone. In some embodiments, the antisense oligomer synergistically increases the susceptibility or sensitivity of a given bacterium to the antimicrobial agent, relative to the antimicrobial agent alone. In some embodiments, the bacterium is Klebsiella pneumoniae, or an MDR strain thereof.

In some embodiments, the antisense oligomer reduces the minimum inhibitory concentration (MIC) of an antimicrobial agent against the bacteria or bacterium being targeted, relative to the antimicrobial agent alone. The “minimum inhibitory concentration” or “MIC” refers to the lowest concentration of an antimicrobial agent that will inhibit the visible growth of a microorganism after overnight (in vitro) incubation. Minimum inhibitory concentrations are important in diagnostic laboratories to confirm resistance of microorganisms to an antimicrobial agent and also to monitor the activity of new antimicrobial agents. The MIC is generally regarded as the most basic laboratory measurement of the activity of an antimicrobial agent against a bacterial organism. Thus, in certain embodiments, the oligomer reduces the minimum inhibitory concentration (MIC) of an antimicrobial agent against the bacteria or bacterium by at least about 5, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800, 900, or 1000% or more (including all integers and ranges in between), relative to the antimicrobial agent alone. In certain embodiments, the oligomer reduces the minimum inhibitory concentration (MIC) of an antimicrobial agent against the bacteria or bacterium by about or at least about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 1000-fold or more (including all integers and ranges in between), relative to the antimicrobial agent alone. In some embodiments, the antisense oligomer synergistically reduces the MIC of an antimicrobial agent against the bacteria or bacterium being targeted, relative to the antimicrobial agent alone. In some embodiments, the bacterium is Klebsiella pneumoniae, or an MDR strain thereof.

### VI. Treatment Monitoring Methods

The efficacy of a given therapeutic regimen involving the methods described herein may be monitored, for example, by general indicators of bacterial infection, such as complete blood count (CBC), nucleic acid detection methods, immunodiagnostic tests, or bacterial culture.

In some aspects, identification and monitoring of bacterial infection involves one or more of (1) nucleic acid detection methods, (2) serological detection methods, i.e., conventional immunoassay, (3) culture methods, and (4) biochemical methods. Such methods may be qualitative or quantitative.

Nucleic acid probes may be designed based on publicly available bacterial nucleic acid sequences, and used to detect target genes or metabolites (i.e., toxins) indicative of bacterial infection, which may be specific to a particular bacterial type, e.g., a particular species or strain, or common to more than one species or type of bacteria (i.e., Gram positive or Gram negative bacteria). Nucleic amplification tests (e.g., PCR) may also be used in such detection methods.

Serological identification may be accomplished using a bacterial sample or culture isolated from a biological specimen, e.g., stool, urine, cerebrospinal fluid, blood, etc. Immunoassay for the detection of bacteria is generally carried out by methods routinely employed by those of skill in the art, e.g., ELISA or Western blot. In addition, monoclonal antibodies specific to particular bacterial strains or species are often commercially available.

Culture methods may be used to isolate and identify particular types of bacteria, by employing techniques including, but not limited to, aerobic versus anaerobic culture, growth and morphology under various culture conditions. Exemplary biochemical tests include Gram stain (Gram, 1884; Gram positive bacteria stain dark blue, and Gram negative stain red), enzymatic analyses, and phage typing.

It will be understood that the exact nature of such diagnostic, and quantitative tests as well as other physiological factors indicative of bacterial infection will vary dependent upon the bacterial target, the condition being treated and whether the treatment is prophylactic or therapeutic.

In cases where the subject has been diagnosed as having a particular type of bacterial infection, the status of the bacterial infection is also monitored using diagnostic techniques typically used by those of skill in the art to monitor the particular type of bacterial infection under treatment.

The PMO or PPMO treatment regimen may be adjusted (dose, frequency, route, etc.), as indicated, based on the results of immunoassays, other biochemical tests and physiological examination of the subject under treatment.

From the foregoing, it will be appreciated how various objects and features of the present disclosure are met. The method provides an improvement in therapy against bacterial infection, for example, multi-drug resistant (MDR) bacteria, using various PPMOs to achieve enhanced cell uptake and anti-bacterial action. As a result, drug therapy is more effective and less expensive, both in terms of cost and amount of compound required.

One exemplary aspect is that compounds effective against virtually any pathogenic bacterial can be readily designed and tested, e.g., for rapid response against new drug-resistant strains.

The following examples are intended to illustrate but not to limit the disclosure. Each of the patent and non-patent references referred to herein is incorporated by reference in its entirety.

### VII. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 0

### Materials and Methods

Peptide-Conjugated Phosphorodiamidate Morpholino Oligomers.

PPMOs were synthesized and purified at Sarepta Therapeutics Inc. (Cambridge, Mass., USA) as previously described (Tilley et al., Antimicrob Agents Chemother 50:2789-2796, 2006). Lyophilized PPMOs were dissolved in ultrapure water and sterile-filtered. PPMO peptides were attached to either the 5′ or 3′ end of the oligomer sequence as indicated.

Bacteria Strains and Growth Conditions.

K. pneumoniae OR1 through OR20 were kindly provided by Karim E. Morey (Oregon Public Health Laboratory, Portland, Oreg.). K. pneumoniae BAA-2146 was obtained from American Type Culture Collection (ATCC, Manassas, Va., USA). K. pneumoniae NDM1-A, -B, -C, and -D was kindly provided by Dr. Susan M. Poutanen (Mount Sinai Hospital, Toronto, ON, Canada). K. pneumoniae 3190, 3290, 3426, and 3427 was kindly provided by Dr. Carl Urban (New York Queens Hospital, NY). K. pneumoniae NIH-1 and NIH-2 were provided by J. Komile Rasheed (CDC, Atlanta, Ga., USA). All other strains were provided by BEI Resources (Manassas, Va.).

Liquid cultures were grown in Mueller-Hinton II (MHII) (cation adjusted) broth. LB agar was used for growth on solid medium. To generate log phase bacteria for in vivo studies, single colonies were cultured aerobically in LB broth overnight (18 h) at 37° C. with shaking (200 rpm). The bacteria were then diluted 4×10⁻² in LB broth and grown at 37° C. with shaking for an additional three hours until the optical density at 595 nm was 0.4, as measured in a 100 μl aliquot in a Costar 3370, 96-well microtiter plate (Corning, NY, USA). The culture was cooled on ice, and the cells washed twice with ice cold PBS by centrifugation (3×10³ g). The washed cells were resuspended in PBS to 1×10⁻² starting volume (about 1×10¹¹ cfu/ml).

Minimal Inhibitory Concentration (MIC) and Minimal Bactericidal Concentration (MBC) Assays.

The MIC was measured by the microdilution method of the Clinical and Laboratory Standards Institute (see, e.g., Standards NCfCL. Methods for Dilution-Antimicrobial Susceptibility Tests for Bacteria That Grow Aerobically; M7-A7; Broth Microdilution Method: CLSI, Wayne, Pa., USA, 2006). Specifically, overnight cultures in MHII were diluted to 5×105 cfu/mL and used to fill wells of a 96-well microtiter plate (Costar 3370). PPMO was added to the first well of each row, and a 2-fold dilution series was made by transferring 100 μl from one well to the next. The plates were incubated on an orbital shaker (200 rpm) at 35-37° C. for 18-20 hours, and then the optical density was measured at 595 nm on a Tecan F500 microplate reader. Samples of each well without visible growth were diluted and spread on LB agar petri dishes. The petri dishes were incubated overnight at 37° C., and the colonies were counted.

Biofilm Assays.

An overnight culture of K. pneumoniae OR5 was diluted 1×10⁻² in LB broth and used to fill (100 μL) wells in four 96-well, non-coated microtiter plates (Costar 3370). The plates were incubated without shaking at 37° C./5% CO₂ for 24 hours. At 24 hours, the liquid cultures were removed from one plate, which was then washed with H₂O and stained with 0.04% crystal violet (Acros Chemical Co., Geel, Belgium) in H₂O. The stained wells were washed with H₂O, and the crystal violet was solubilized with 95% ethanol. The solubilized stain was measured in an ELISA plate reader at 595 nm. A second plate was used to measure the viable cells in the biofilm at 24 hours. The liquid cultures were removed and the biofilm was washed with H₂O. The biofilm was solubilized by adding 100 μL 0.1% Triton X-100, incubating at 22° C. for 15 min, and then mixing with a pipette. Conditions to fully solubilize the viable cells were optimized in prior experiments, and it was found that Triton X-100 resulted in the solubilization of the maximal number of cells. Other detergents were less effective than Triton X-100, and mild sonication in a water bath did not increase the viable cell count. The solubilized biofilm was diluted in PBS, spread on LB agar petri dishes, incubated 18-24 h at 37° C., and the colonies counted. The liquid cultures from the remaining 2 microtiter plates were removed at 24 hours, washed with H₂O, and the wells refilled with 100 μL of LB broth or PBS without or with various concentrations of PPMO. The plates were again incubated without shaking for at 37° C./5% CO₂ for an additional 24 hours, and then processed.

MBEC Biofilm Assays and Confocal Microscopy.

K. pneumoniae strain OR05 was incubated for 6 hours in MHII, adjusted to an O.D. 600 nm of 0.08 in DPBS (1×108 cfu/ml), then diluted to 5×105 cfu/ml in MHII. A 96-well MBEC plate (Innovotech, Alberta, Canada) was inoculated with 150 μl per well of this bacterial working solution, then incubated at 37° C., 110 rpm, for 24 hours. The pegs were transferred to a plate with fresh medium containing serial two-fold dilutions of PPMO, or no treatment, and incubated for an additional 24 hours. The pegs were then processed by crystal violet assay, for colony forming units, or for confocal microscopy.

For crystal violet assay, MBEC pegs were processed as previously described (see Howard et al., Antimicrob Agents Chemother 2017; 61). Assays were run in triplicate, with 2-3 wells per condition per assay. For colony determination, pegs were washed three times in DPBS, cut at the base with a heated scalpel, and placed in round bottom tubes containing 1 ml DPBS. The tubes were sonicated in a room temperature water bath for 20 minutes, then serially diluted and drip-plated for colony forming units. Assays were run in quadruplicate, with 2-3 pegs per condition per assay.

For spinning disk confocal microscopy, the MBEC pegs were washed for 1 minute in 150 mM NaCl, cut near the base with a heated scalpel, stained with Live/Dead BacLight Bacterial Viability Kit stain (Thermo Fisher Scientific) (3 μl Syto 9: 3 μl Propidium Iodide: 994 μl 150 mM NaCl) for 15 minutes, and rinsed with 150 mM NaCl. Pegs were then imaged on an Axiovert 200 M inverted microscope (Carl Zeiss, Thornwood, N.J.) with an UltraVIEW ERS spinning-disk confocal head (Perkin-Elmer, Waltham, Mass.) using 488 nm and 568 nm line lasers. Z-stacks were visualized sequentially in the TRITC and FITC channels with a 40×/NA 1.3 oil immersion objective. Images were volumetrically rendered in IMARIS software (Bitplane, Concord, Mass.).

Mouse Survival Experiments.

Female Balb/c mice, aged 6-8 weeks (Jackson Labs, Sacramento, Calif., USA) were randomly assigned to treatment groups, and then treated with 3 mg cyclophosphamide in 100 μl PBS by intraperitoneal injection one and four days prior to infection. Mice were anesthetized by isoflurane and infected intranasally with ˜3.0×10⁷ cfu of K. pneumoniae NR15410 in 25 μl PBS. Treatment with various doses of PPMO (as indicated in figure legend) was initiated 0, 8, 24 or 48 hours post infection by intranasal administration of 25 μl, and then repeated twice at 24 hour intervals (each mouse received a total of 3 doses PPMO). Body temperature was measured in the ear using an infrared thermometer (Braun Thermoscan Pro 4000, Kaz USA, Marlborough, Mass.).

Mouse Lung burden.

Six-eight week old female Balb/c mice (Jackson Laboratories, Bar Harbor, Me.) were treated with 3 mg cyclophosphamide in 100 μl PBS by intraperitoneal injection one and four days prior to infection. Mice were anesthetized by isoflurane and infected with 2.7-2.9e7 CFU of K. pneumonia NR15410 (in 25 μL) along with 300 μg of the indicated PPMO or H₂0 control. Six hours post-infection, mice were anesthetized by isoflurane and treated intranasally with 300 μg of the indicated PPMOs or H₂0 in a 25 μL volume. Mice were euthanized 24 hours post-infection and whole lungs were collected for CFU enumeration. Lungs were weighed and homogenized for 10 seconds in 1 mL of PBS followed by serially dilution and plating for CFU enumeration.

Graphical Software and Statistical Analysis.

Standard deviation and graphical analysis was performed on GraphPad Prism®6 software (GraphPad Software, Inc., San Diego, Calif., USA). Treatment group means were compared by t-test (unpaired, two-tailed, Mann-Whitney). All cfu data were transformed to log values prior to analysis.

### Example 1

### Activity of PPMOs Targeted Against Expression of Essential Genes of K. pneumoniae

PPMOs were designed and synthesized with 11 nucleobases complementary to the regions near the start codon of 8 genes that are essential in K. pneumoniae or other bacteria. All PPMOs had the peptide ((RXR)₄XB) attached to the 5′-end, except two (0802 and 0017) that used R₆G. R₆G conjugates had been tested only in Pseudomonas aeruginosa and not in Klebsiella (see, e.g., Howard et al., Antimicrob Agents Chemother 2017; 61; and Sully et al., J Antimicrob Chemother 2017; 72: 782-90). Three of the PPMOs targeted different positions on acpP: one (0276) spanning the start codon and two (0076 and 0802) starting 3 bases downstream of the start codon.

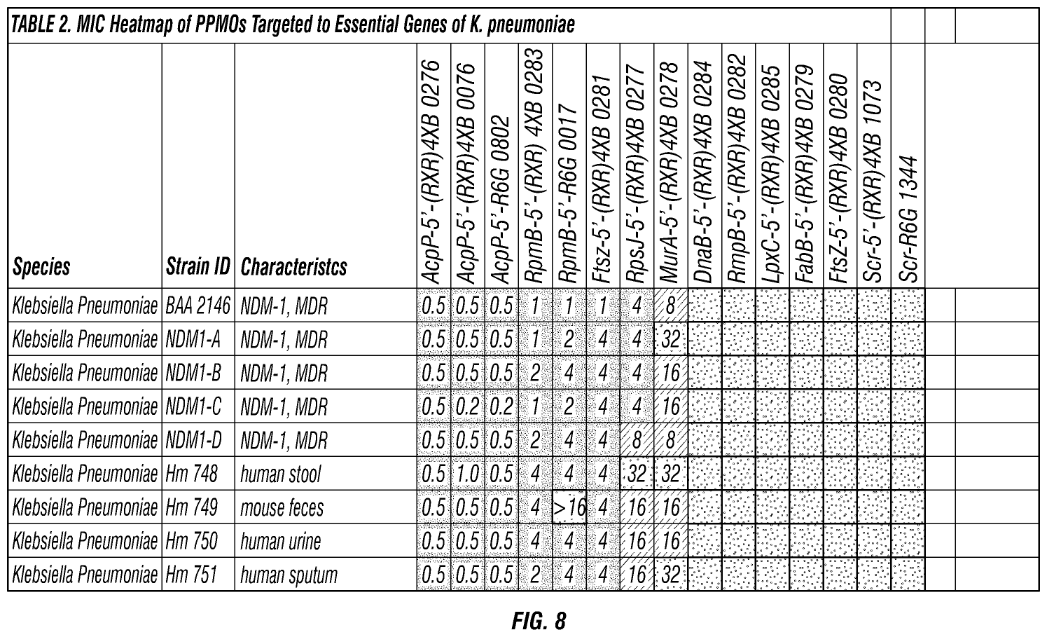

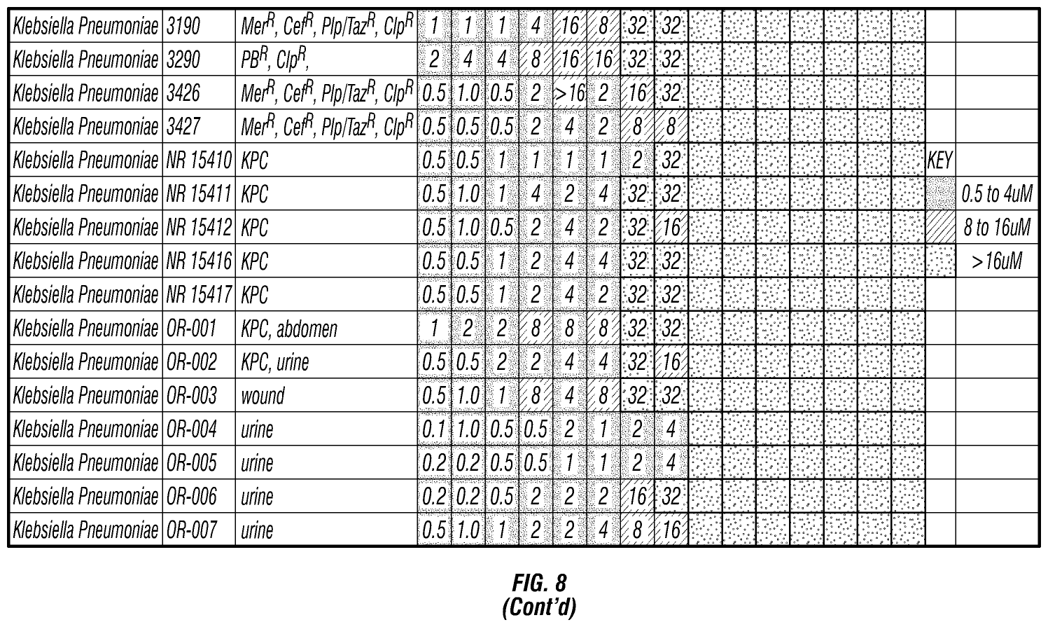

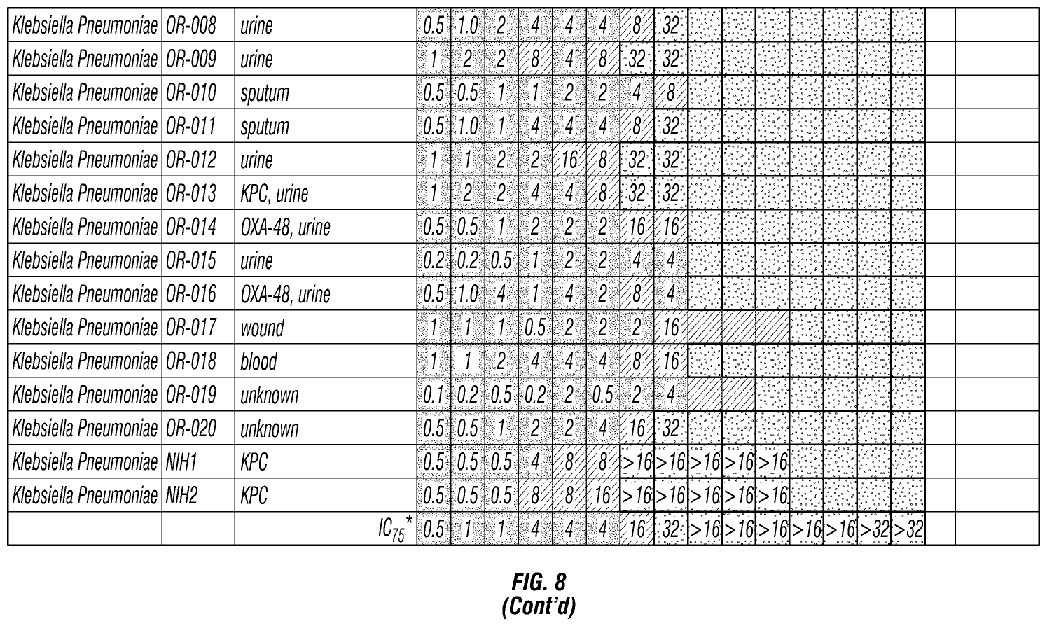

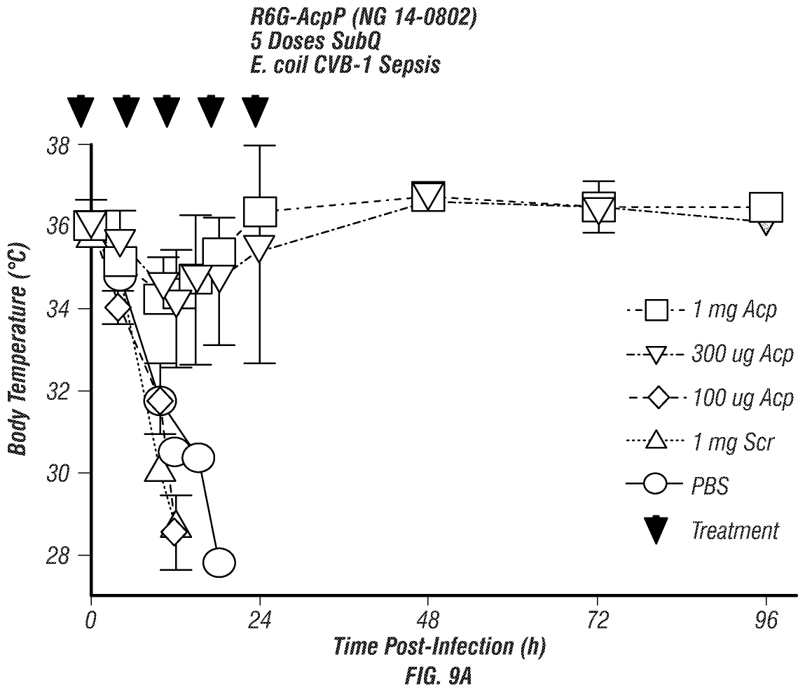

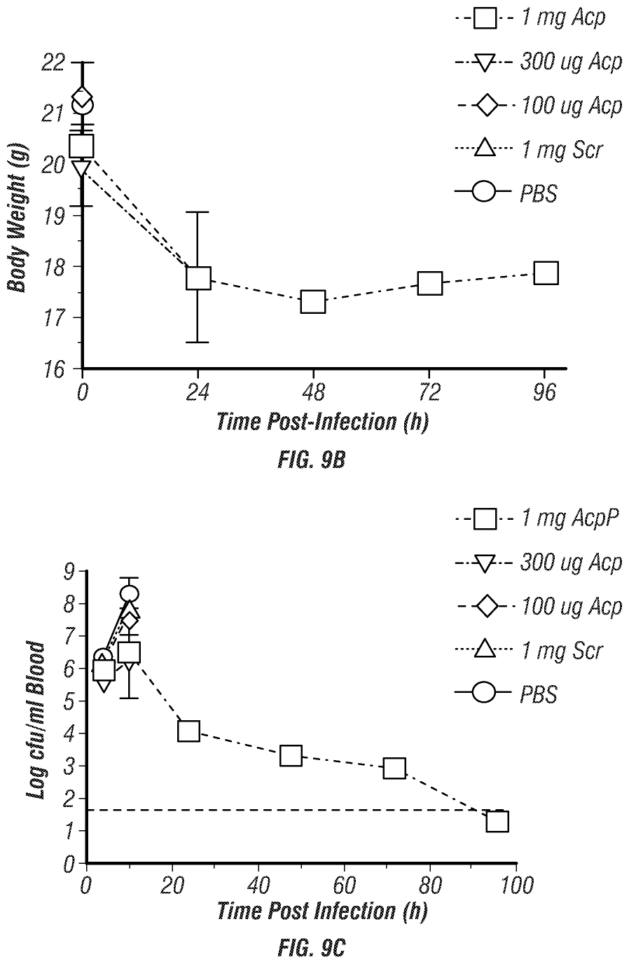

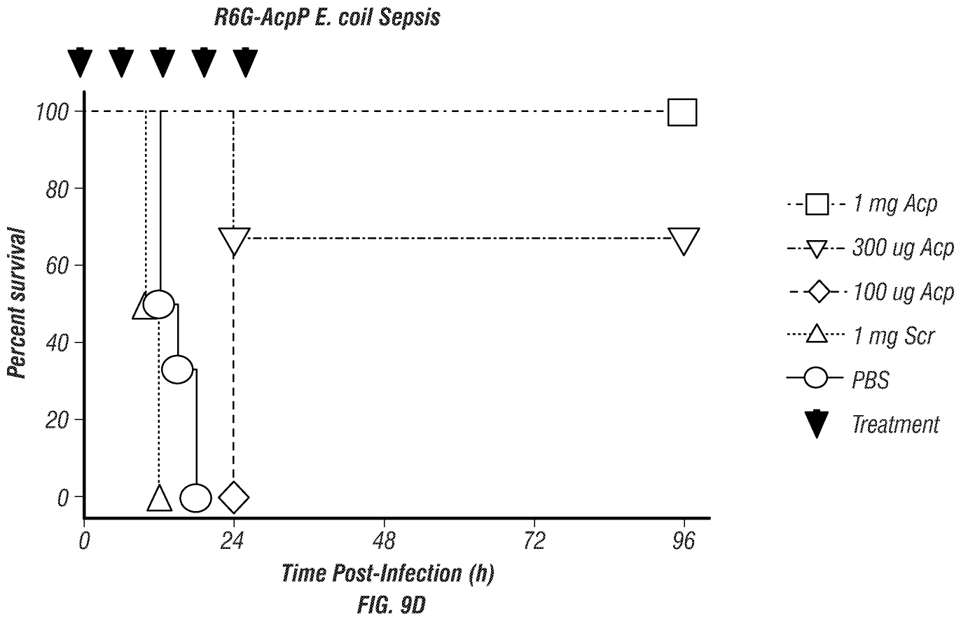

The MIC of all PPMOs was measured using a panel of 40 strains of K. pneumoniae. The results show that the 5′-(RXR)₄XB-AcpP PPMO (0276) was the most potent with an IC₇₅=0.5 μM (see Table E1 and FIG. 8; IC₇₅ is the minimal inhibitory concentration that prevented growth of at least 75% of the strains). This was followed closely by two other PPMOs targeted to acpP (0076 and 0802) with an IC₇₅=1.0 μM, and 3 PPMOs targeted to rpmB and ftsZ with IC₇₅=4 μM.

To optimize the PPMO for use with K. pneumoniae, a set of AcpP PPMOs was synthesized with different peptides composed of arginine (R) and tyrosine (Y), glycine (G), or phenylalanine (F) instead of 6-aminohexanoic acid (X). The MIC of each PPMO was measured in the panel of strains (see Table E1 and FIG. 8).

Another feature of the PPMOs was assessed, which is the position of the conjugated peptide (5′ or 3′ end of PPMO). Seven PPMOs (0948, 0155, 0398, 0399, 0099, 0141, and 0205) were synthesized with the same base sequence and peptides as 0076, 0802, 0276, 0016. 0605, 0606, and 0621, respectively, except the peptides were attached to the 3′-end. The PPMOs were tested by measuring the MIC in the panel of 40 strains. The results show that two of the 3′-conjugates were 2-fold less potent than their 5′-equivalents, but there was no difference in any of the other pairs (Table E1).

TABLE E1

| PPMO no. | Target | MIC₇₅ |
|---|---|---|
| 0076 | acpP +6 to +16 | 1 |
| 0948 | acpP +6 to +16 | 1 |
| 0802 | acpP +6 to +16 | 1 |
| 0155 | acpP +6 to +16 | 2 |
| 0276 | acpP −4 to +6 | 0.5 |
| 0398 | acpP −4 to +6 | 1 |
| 0016 | acpP −4 to +6 | 2 |
| 0399 | acpP −4 to +6 | 2 |
| 0605 | acpP +6 to +16 | 1 |
| 0099 | acpP +6 to +16 | 1 |
| 0606 | acpP +6 to +16 | 1 |
| 0141 | acpP +6 to +16 | 1 |
| 0621 | acpP +6 to +16 | 1 |
| 0205 | acpP +6 to +16 | 1 |
| 1073 | scrambled | >32 |
| 1344 | scrambled | >32 |

Minimal Bactericidal Concentration (MBC).

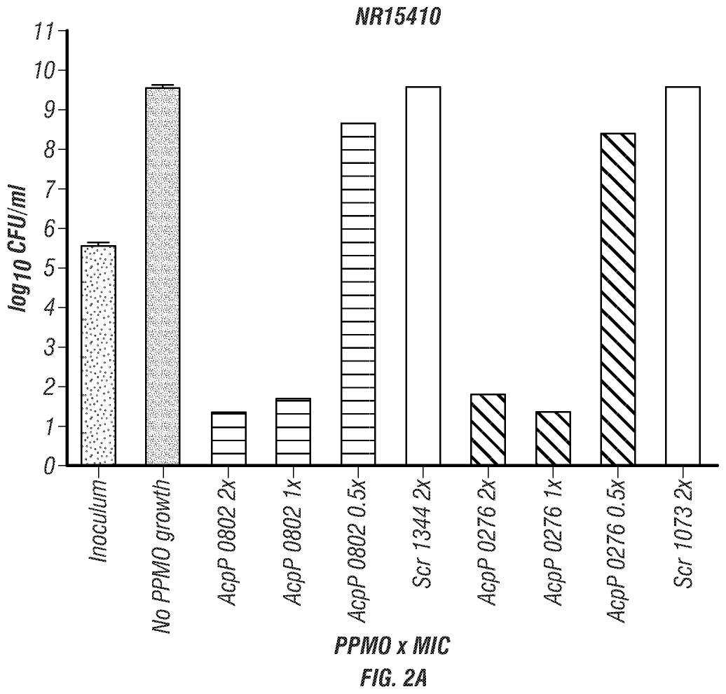

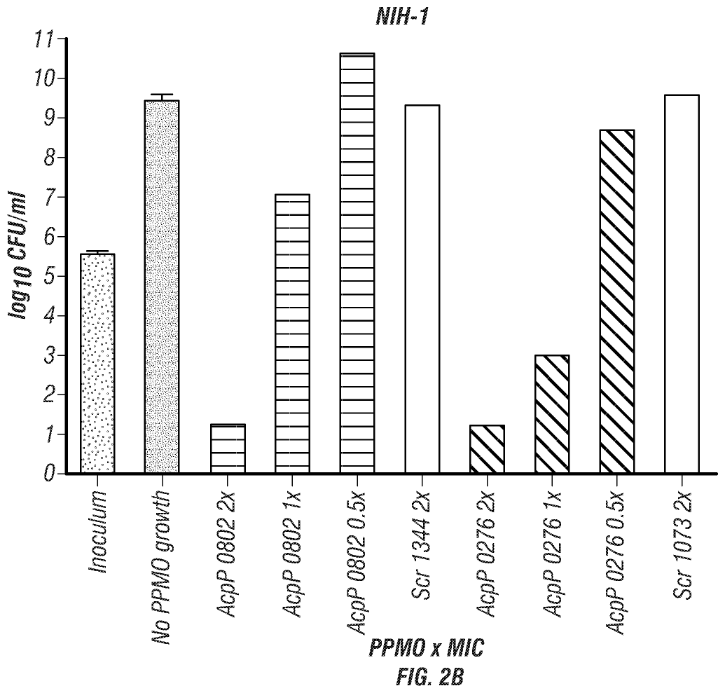

The MBC of 0276 and 0802 were measured in 2 strains from the panel of K pneumoniae, NR15410 and NIH-1. The results show that either PPMO was bactericidal at 2×MIC in either strain (see FIG. 2A-B). 0276 was also bactericidal at 1×MIC in either strain, whereas 0802 was bactericidal at 1×MIC in only NR15410. Scr PPMOs had no effect on viability at any concentration tested.

### Example 2

### Activity of PPMOs Against Biofilms

PPMOs were tested for their ability to reduce established biofilms and viable cells within biofilms. Biofilms were established in microtiter plates by growing cultures of K. pneumoniae for 24 hours. Following the establishment of biofilms, the spent medium was removed and replaced with fresh broth plus 8, 16, or 32 μM AcpP-PPMO (0276 or 0802), and grown for another 24 hours. Control cultures included either no PPMO or 8, 16, or 32 μM scrambled base (Scr) PPMO (1073 or 1344). The biofilm mass and viable cells in the biofilms were then measured.

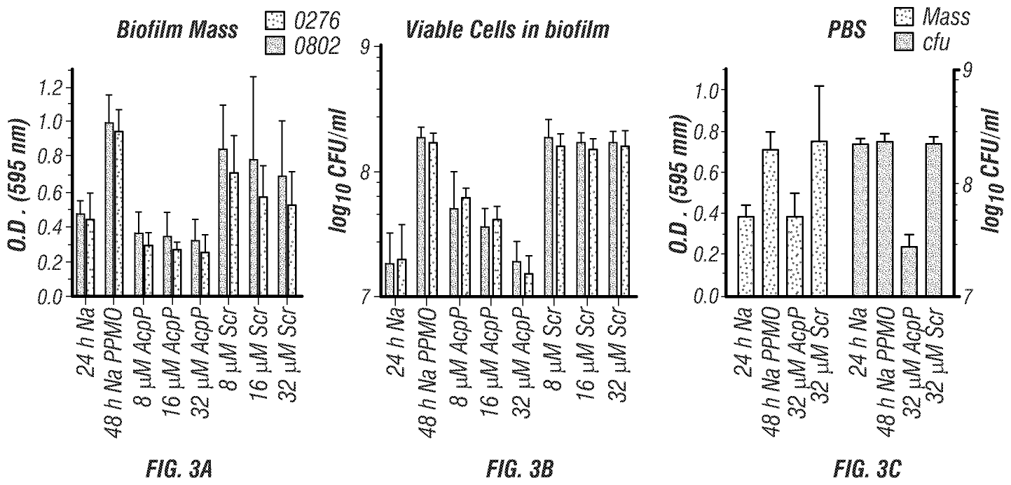

The results show that 32 μM PPMO 0276 or 0802 reduced the established biofilm mass by 32% and 47%, respectively, compared to the 24 hour biofilm (FIG. 3A). Furthermore, treatment with 32 μM PPMO 0276 or 0802 resulted in a 68% and 74% reduction, respectively, compared to the 48 hour culture without PPMO. Lower concentrations of the AcpP PPMOs resulted in a proportionally lower reduction in biofilm mass, but still significantly less biofilm than the untreated culture. The Scr PPMO did not reduce biofilm at any concentration tested compared to the untreated culture.

Treatment of established biofilms with the AcpP PPMOs also reduced the number of viable cells in the biofilm. Treatment with 8, 16, or 32 μM AcpP PPMO 0276 resulted in significantly fewer viable cells in the biofilm compared to the untreated culture (5.6×10⁷, 3.8×10⁷, or 2.0×10⁷ cfu/ml, respectively, compared to 1.9×10⁸ cfu/ml in the untreated culture at 48 hours (FIG. 3B). Similar reductions in viable cells were observed with the AcpP PPMO 0802 (FIG. 3B). Neither Scr PPMO had any effect on viability of cells within the biofilm (FIG. 3B).

The reduction in viable cells in the biofilm suggested that the PPMOs penetrated the biofilm. However, another mechanism might account for the reduction: fewer viable cells in the planktonic culture might have led to fewer cells entering the biofilm, resulting in a reduction in both biofilm mass and viable cells within the biofilm. To distinguish between these two possibilities, biofilms were established for 24 hour without PPMO, as before. After establishing the biofilm, the planktonic culture was removed and replaced with PBS instead of rich growth medium. AcpP or Scr PPMO was added, and biofilm was incubated for another 24 hours. The results show that the biofilm mass treated with the AcpP PPMO in PBS remained the same as the mass at 24 hours, whereas the mass nearly doubled in the untreated or Scr PPMO-treated biofilms (FIG. 3C). More significantly, viable cells within the biofilm which was treated with AcpP PPMO in PBS decreased by 88% from 24 hours, whereas viable cells in the untreated or Scr PPMO-treated biofilms remained unchanged.

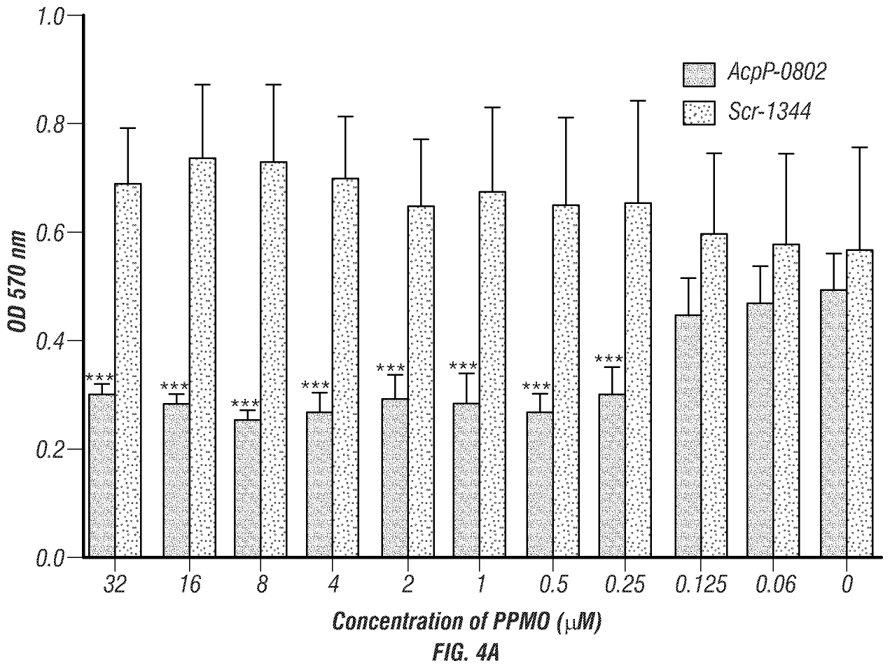

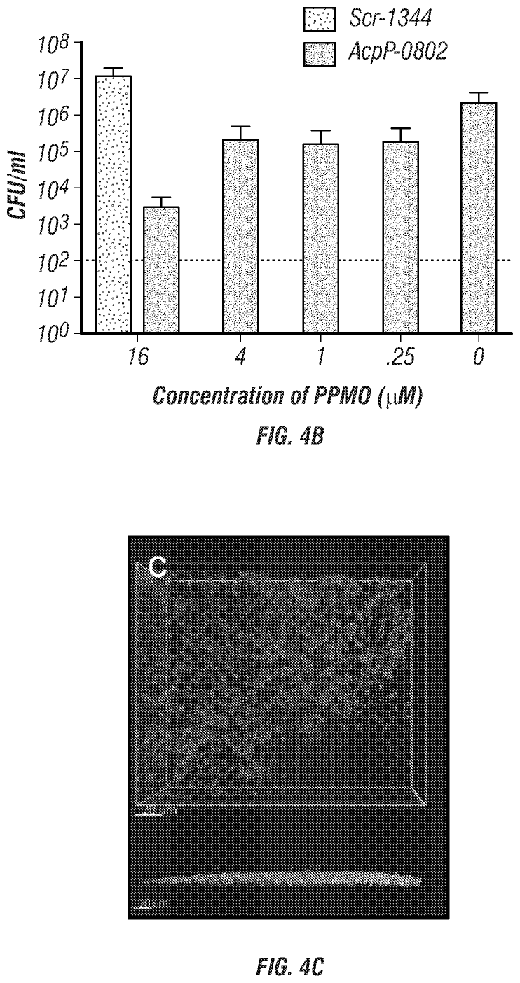

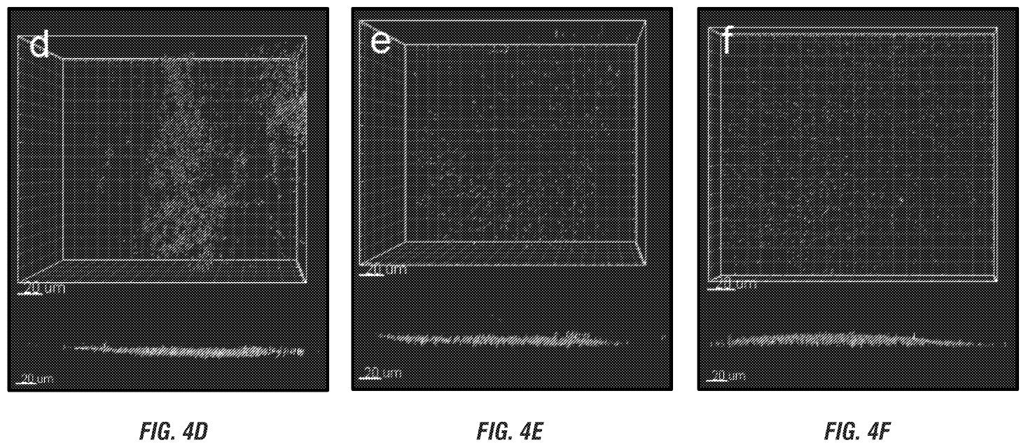

PPMO penetration of biofilm was also measured using an MBEC (minimum biofilm eradication concentration) inoculator system. The K. pneumoniae OR5 culture was inoculated into a 96-well MBEC plate and biofilm grew for 24 hours, with aeration, on the MBEC inoculator pegs. The spent broth was replaced with fresh broth containing AcpP PPMO (0802) or Scr PPMO (1344) serially diluted two-fold from 32 μM to 0.06 μM, or no treatment, and incubated for an additional 24 hours. The pegs were then processed by crystal violet assay for biomass (FIG. 4A), by sonication for colony forming units (FIG. 4D), or for imaging by confocal microscopy (FIGS. 4C-F).

As for the stationary 96-well plate assay, treatment with 32, 16, and 8 μM AcpP PPMO (0802) resulted in a significant reduction (p<0.0001) in biofilm mass as compared to Scr PPMO (1344) (FIG. 4A). This significance was evident down to a concentration of 0.25 μM PPMO. Bacterial counts demonstrate that 16 μM AcpP PPMO (0802) treatment resulted in a 3-log reduction (99.9%) in viable cells compared to untreated control, while 0.25 μM treatment resulted in a 91.9% reduction (FIG. 4B). Scr PPMO (1344) had no effect on cell viability. Confocal imaging of live/dead stained pegs further illustrated that treatment with 16 or 2 μM AcpP PPMO (FIGS. 4E-F) dramatically reduced cellular biomass in comparison to untreated and scrambled controls (FIGS. 4B-C). Taken together, this independent approach supports the same conclusion shown in FIGS. 3A-C, that PPMOs effectively penetrate Klebsiella biofilms.

### Example 3

### Activity of PPMOs In Vivo

The efficacy of the R₆G-AcpP PPMO (0802) was evaluated in vivo using a mouse pneumonia model. Mice were infected intranasally with the multidrug-resistant clinical isolate K. pneumoniae NR15410, and then treated intranasally once daily for 3 days with various amounts of PPMO from 0.6 to 30 mg/kg. Control groups were infected and treated similarly with either PBS or a scrambled sequence PPMO (Scr).

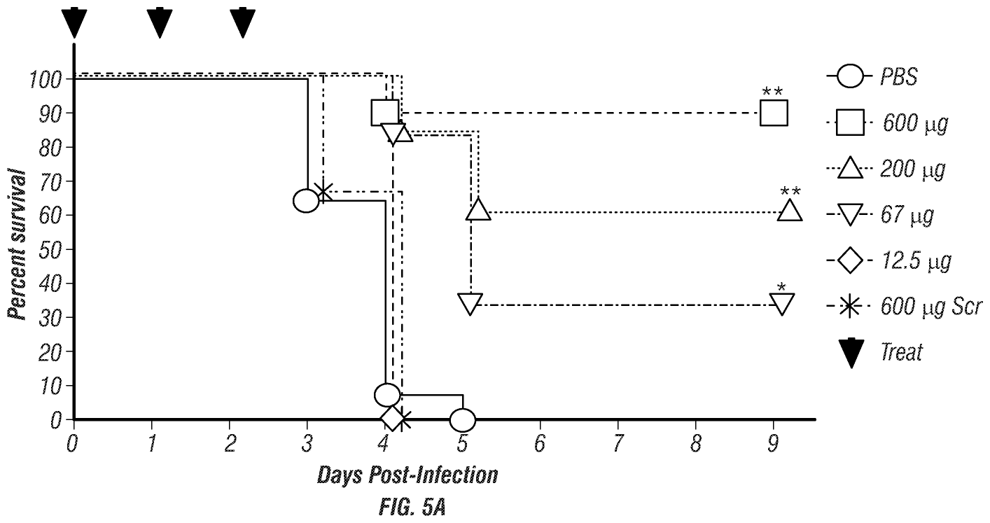

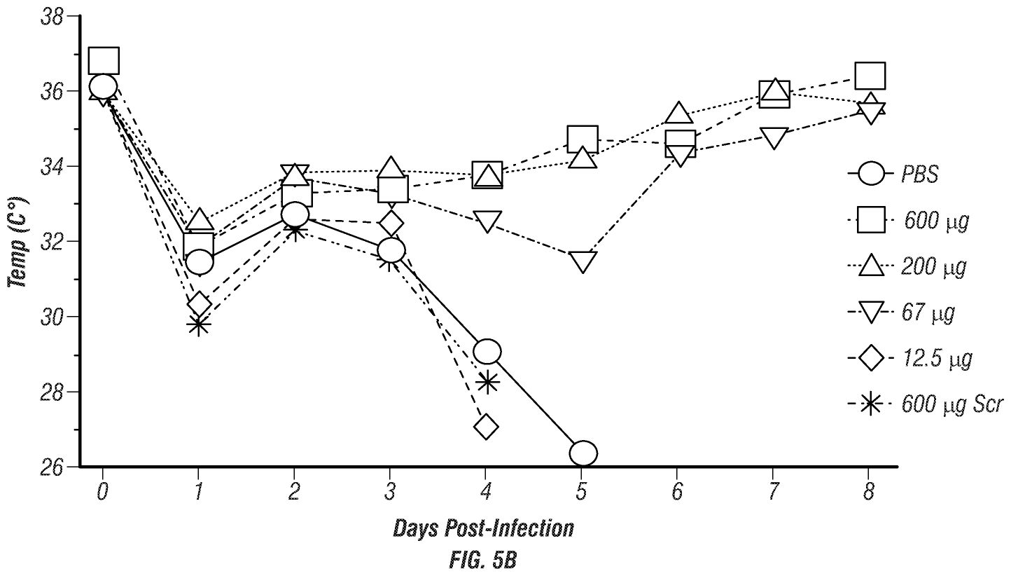

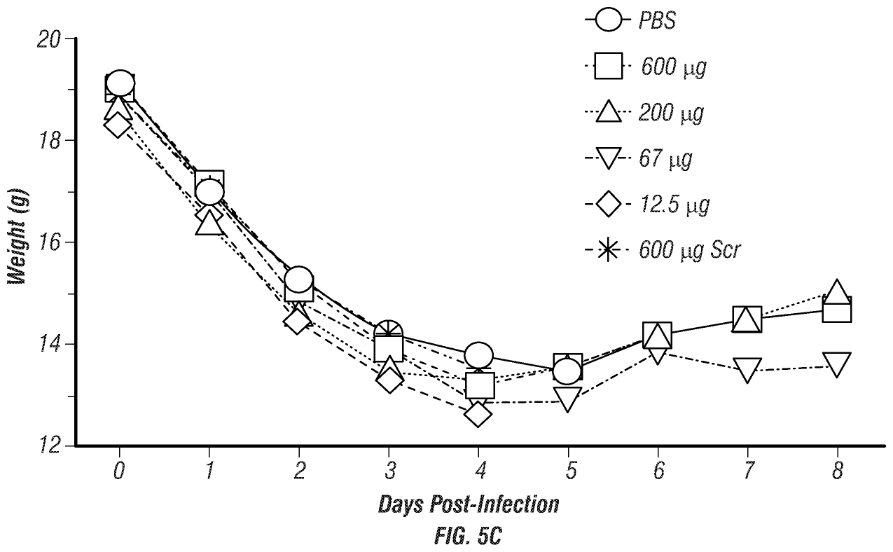

The results show dose-dependent responses. Survival was 89%, 62%, and 33% for mice treated with 30, 10, and 3.3 mg/kg of R₆G-AcpP PPMO, respectively (FIG. 5A). This was significantly greater (p<0.002) than mice treated with either PBS or the Scr PPMO. All mice treated with lower amounts of the AcpP PPMO, PBS, or 30 mg/kg Scr PPMO died by day 5 post-infection. Surviving mice began to regain weight by 4 to 5 days post-infection, whereas non-survivors continually lost weight (FIG. 5B). Similarly, body temperature in surviving mice began to recover between 3 to 5 days post-infection, whereas it continued to decrease after day 2 or 3 in all mice that ultimately did not survive (FIG. 5C).

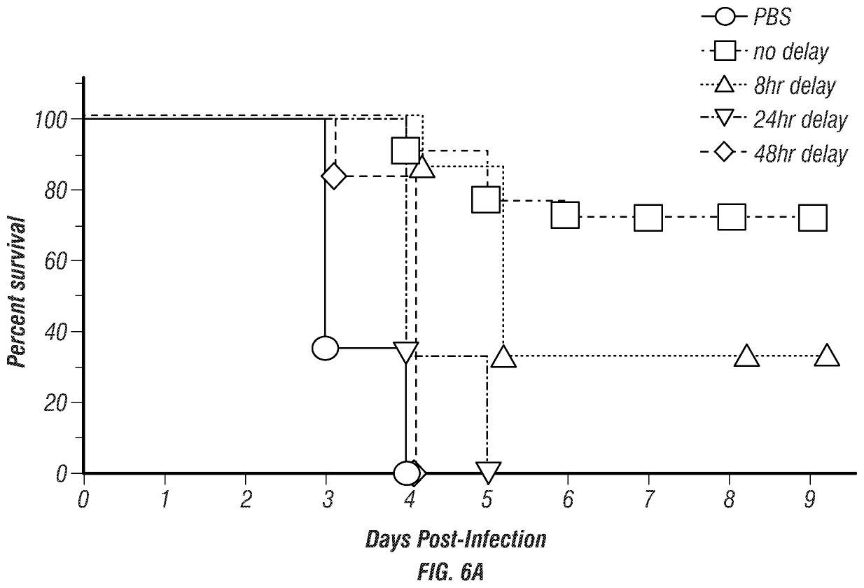

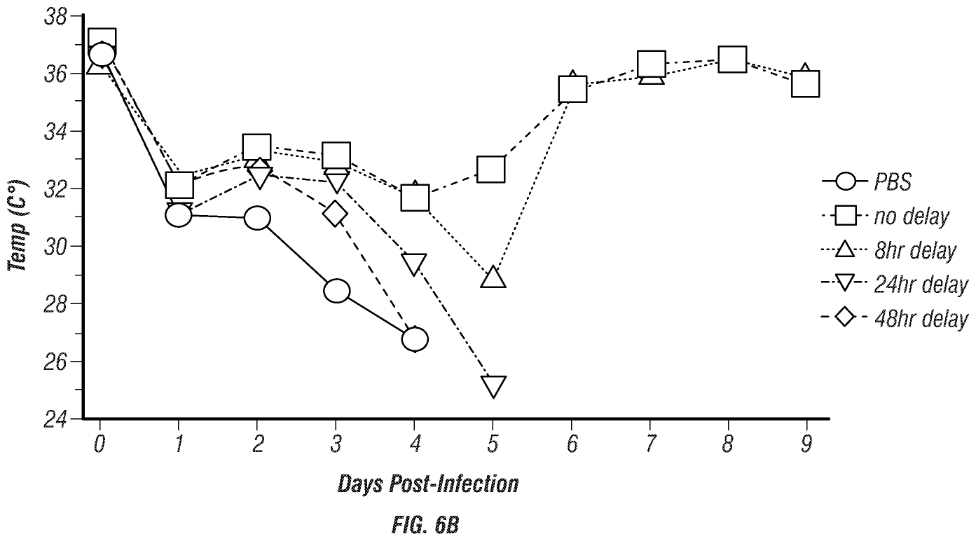

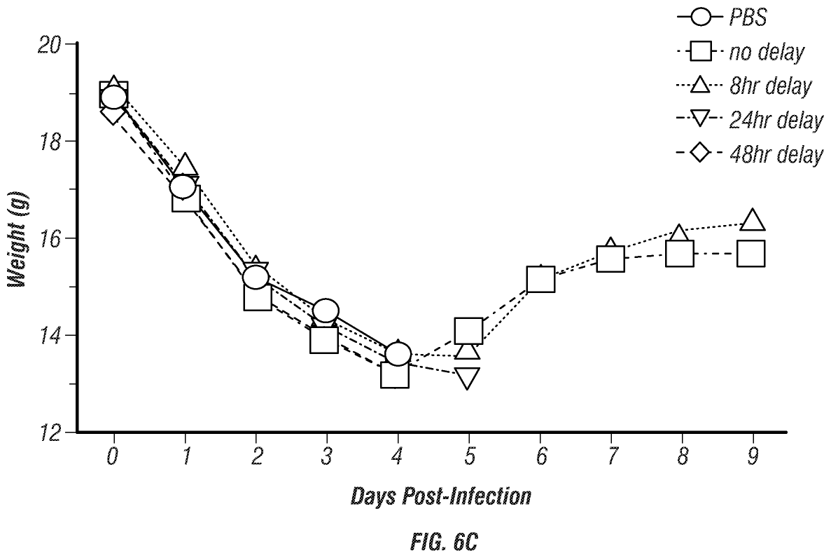

The R₆G-AcpP PPMO (0802) was also tested therapeutically by administering it post-infection. Groups of mice were infected as described above and then treated with the first dose of 600 μg (˜30 mg/kg) PPMO at 0, 8, 24, or 48 hours post-infection. Each group was treated two more times at 24 h intervals. When treatment was not delayed, 71% of the mice survived (FIG. 6A). Delaying treatment for 8 hours resulted in 33% survival, which is significantly greater than the PBS control group. There were no survivors in the PBS-treated group or when initial treatment was delayed for 24 or 48 hours. However, median survival time in the 8 hour (5.0 d) and 24 hour (4.0 d) delayed treatment groups was significantly (p<0.01) greater than that in the PBS-treated group (3.0 d). Body temperature decreased less in mice treated initially at 0 or 8 hours compared to groups treated initially at 24 or 48 hours, or with PBS (FIG. 6B). All surviving mice started to regain weight between 4 to 5 d post-infection (FIG. 6C).

The bacterial burden in the lungs of infected mice was measured. Groups of mice were infected intranasally and then treated intranasally once with 300 μg/kg R₆G-AcpP PPMO (0802), R₆G-Scr PPMO (1344), or PBS. After 24 hours post-infection, lungs were removed, homogenized and spread on petri dishes to determine viable bacteria.

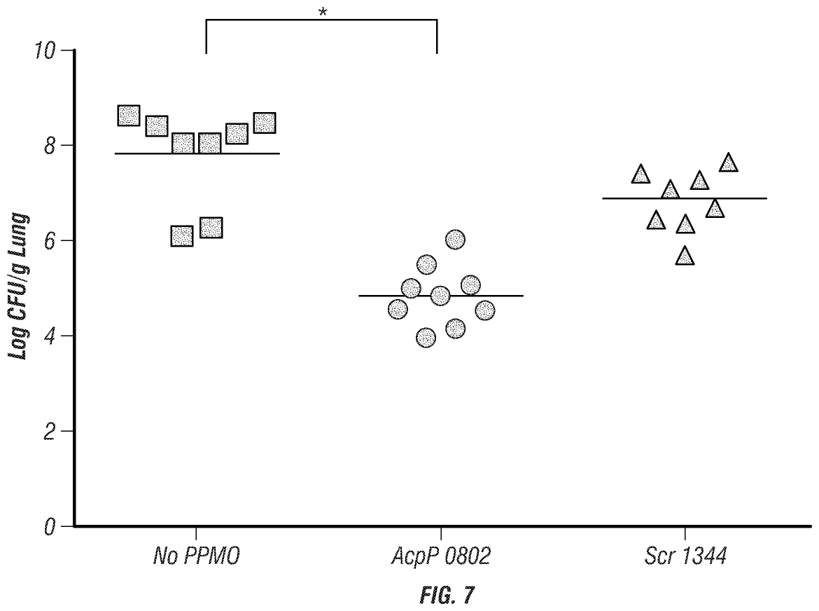

The results show a highly significant (p<0.0001), 3-log reduction in viable bacteria from the lungs of mice treated with AcpP PPMO 0802 compared to the mice treated with PBS (FIG. 7). No statistically significant (p>0.05) reduction was found in the group treated with Scr PPMO (1344) compared to PBS-treated mice, although there was a trend toward a 1-log reduction.

### Example 4

### Discovery of Multipathogen acpP PPMOs

E. coli and Klebsiella are both frequent gram-negative causes of infections of the urinary tract. Although the majority of PPMOs are designed to be pathogen specific, given the closer phylogenetic relationship of E. coli and Klebsiella, this work has resulted in the discovery of the first “bi-functional” multipathogen acpP PPMO (SEQ ID NOs: 1 and 2). These PPMOs have been found to have both in vitro and in vivo activity in both pathogens and therefore these PPMOs could be used empirically in suspected or confirmed urinary tract infections due to either of these pathogens (MIC Table 4)

TABLE 4
MIC Table of a bi-functional multipathogen acpP PPMO. Column
numbers indicate PPMO variations as outlined in Table 2A.

| Strain | 0155 | 0399 | 0948 | 0398 | 0802 | 0016 | 0076 | 0276 |
|---|---|---|---|---|---|---|---|---|
| E. coli AIS 070834 | 1 | 1 | 0.5 | 0.5 | 1 | 0.5 | 0.5 | 0.5 |
| E. coli CVB-1 | 2 | 2 | 0.25 | 1 | 4 | 1 | 1 | 1 |
| E. coli NDM1-E | 1 | 1 | 0.5 | 1 | 1 | 0.5 | 0.25 | 0.5 |
| E. coli W3110 | 4 | 4 | 2 | 4 | 2 | 2 | 2 | 2 |
| K. pneumoniae15410 | 1 | 1 | 0.5 | 0.5 | 0.5 | 1 | 0.5 | 0.5 |
| K. pneumoniqe 15411 | 2 | 4 | 2 | 1 | 1 | 2 | 1 | 0.5 |
| K. pneumoniae NDM1-A | 1 | 2 | 0.5 | 0.5 | 0.5 | 1 | 0.5 | 0.5 |
| K. pneumoniae OR1 | 4 | 4 | 2 | 2 | 2 | 2 | 2 | 1 |

### Example 5

### Discussion

The results described herein show that PPMOs targeted to essential genes in K. pneumoniae inhibited growth in pure cultures of a panel of 44 diverse strains. The MIC75 values varied from 0.5 μM to >16 μM. The MBC of two PPMOs targeted to acpP were either 1 or 2×MIC, depending on the strain used for testing. PPMOs also retained their activity in MDR strains of Klebsiella.

The PPMOs were also able to reduce both established biofilm mass and viability of K. pneumoniae within the biofilm. Biofilms protect embedded bacteria by reducing the effectiveness of antibiotics, bacteriophage and the immune system (Anderl et al., Antimicrob Agents Chemother 2000; 44: 1818-24). Although the exact mechanism(s) of biofilm protection against antibiotics is controversial, it seems dependent on the bacterium and the structures of the biofilm and antibiotic, and not necessarily related to diffusion limitations linked to molecular weight (Singh et al., Pathogens and disease 2016; 74; and Zahller and Stewart, Antimicrob Agents Chemother 2002; 46: 2679-83). The results are consistent with a hypothesis that Klebsiella biofilm does not pose a diffusion limitation, since the molecular weight of PPMOs is about 10 to 20 times that of most common antibiotics. Although the mechanism by which PPMOs reduce biofilm mass and gain access to kill the bacteria is unknown, the results from treating the established biofilm with PPMO in PBS are consistent with the PPMO penetrating the biofilm to gain access to the pathogen. The results are inconsistent with the PPMO acting only on the planktonic cells. In addition, the results are inconsistent with the membrane-penetrating peptide portion of the PPMO simply solubilizing the biofilm, since a scrambled base sequence PPMO with the same peptide attached did not reduce biofilm mass. These data suggest that PPMOs penetrate Klebsiella biofilms, kill the cells within the biofilm, and reduce biofilm mass.

These results also provide the first vetted report of the effectiveness of an antisense oligomer against K. pneumoniae in an animal model of infection. Here, the results show that an AcpP PPMO improved survival in a dose-dependent manner and in a dosage range that is clinically achievable (3 to 30 mg/kg/dose once per day). In addition, other objective measurements of health, including body temperature and weight, paralleled the results of survival and reinforced the effectiveness of the PPMO.

The scrambled PPMO (1344) showed a slight, but statistically insignificant reduction of bacterial lung burden. However, this was not sufficient to increase survival. No effect of the Scr PPMO was found in any of the in vitro assays.

These results have established the in vivo efficacy of a gene-specific therapeutic targeted to an essential gene in the highly antibiotic resistant K. pneumoniae. This PPMO reduced morbidity and increased survival in a mouse model of pneumonia. The PPMO was well-tolerated in vivo, and was effective when used therapeutically at clinically-achievable doses. PPMOs thus have the potential for clinical applications against multidrug resistant pathogens.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. An antisense morpholino oligomer, composed of morpholino subunits and phosphorus-containing intersubunit linkages joining a morpholino nitrogen of one subunit to a 5′-exocyclic carbon of an adjacent subunit, and having (a) about 10-40 nucleotide bases, and (b) a targeting sequence of sufficient length and complementarity to specifically hybridize to a bacterial mRNA target sequence that encodes a protein associated with a biochemical pathway and/or cellular process, where the oligomer is conjugated to a cell-penetrating peptide (CPP).

2. The antisense morpholino oligomer of formula (I): 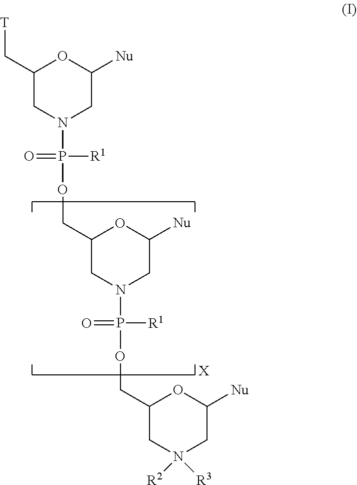
or a pharmaceutically acceptable salt thereof,
where each Nu is a nucleobase which taken together forms a targeting sequence;
X is an integer from 9 to 38;
T is selected from OH and a moiety of the formula: 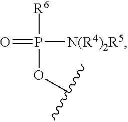
where each R⁴ is independently C₁-C₆ alkyl, and R⁵ is selected from an electron pair and H, and R⁶ is selected from OH, —N(R⁷)CH₂C(O)NH₂, and a moiety of the formula: 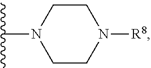
where:
R⁷ is selected from H and C₁-C₆ alkyl, and
R⁸ is selected from G, —C(O)—R⁹OH, acyl, trityl, and 4-methoxytrityl, where:
R⁹ is of the formula —(O-alkyl)_{y}- where y is an integer from 3 to 10 and each of
the y alkyl groups is independently selected from C₂-C₆ alkyl;
each instance of R¹ is —N(R¹⁰)₂R¹¹ where each R¹⁰ is independently C₁-C₆ alkyl, and R¹¹ is
selected from an electron pair and H;
R² is selected from H, G, acyl, trityl, 4-methoxytrityl, benzoyl, stearoyl, and a moiety of the formula: 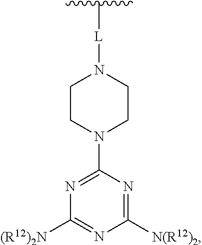
where L is selected from —C(O)(CH₂)₆C(O)— and —C(O)(CH₂)₂S₂(CH₂)₂C(O)—, and each R¹² is of the formula —(CH₂)₂OC(O)N(R¹⁴)₂ where each R¹⁴ is of the formula —(CH₂)₆NHC(═NH)NH₂; and
R³ is selected from an electron pair, H, and C₁-C₆ alkyl,
where G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,
and —C(O)CH₂NH—CPP, or G is of the formula: 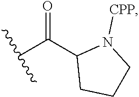
where the CPP is attached to the linker moiety by an amide bond at the CPP carboxy terminus, with the proviso that only one instance of G is present,
where the targeting sequence specifically hybridizes to a bacterial mRNA target sequence that encodes a protein associated with a biochemical pathway and/or cellular process.

3. The antisense morpholino oligomer of claim 1, wherein the targeting sequence specifically hybridizes to a Klebsiella pneumonia mRNA target sequence that encodes a protein associated with a biochemical pathway and/or cellular process.

4. The antisense morpholino oligomer of claim 1, where the target sequence comprises a translational start codon of the bacterial mRNA and/or a sequence within about 30 bases upstream or downstream of the translational start codon of the bacterial mRNA.

5. The antisense morpholino oligomer of claim 1, where the protein associated with a biochemical pathway and/or cellular process is a fatty acid biosynthesis protein.

6. The antisense morpholino oligomer of claim 5, where the fatty acid biosynthesis protein is an acyl carrier protein encoded by acpP.

7. The antisense morpholino oligomer of claim 5, where the fatty acid biosynthesis protein is an acyl carrier protein synthase encoded by fabB.

8. The antisense morpholino oligomer of claim 1, where the targeting sequence is set forth in SEQ ID NOS:1-3, comprises a fragment of at least 10 contiguous nucleotides of SEQ ID NOS: 1-3, or comprises a variant having at least 80% sequence identity to SEQ ID NOS: 1-3, where thymine bases (T) are optionally uracil bases (U).

9. The antisense morpholino oligomer of claim 8, which is selected from Table 2A.

10. The antisense morpholino oligomer of claim 1, where the protein associated with a biochemical pathway and/or cellular process is a UDP-N-acetylglucosamine 1-carboxyvinyltransferase peptidoglycan biosynthesis protein encoded by murA.

11. The antisense morpholino oligomer of claim 1, where the protein associated with a biochemical pathway and/or cellular process is a ribosomal protein.

12. The antisense morpholino oligomer of claim 11, where the ribosomal protein is a 50S ribosomal protein L28 encoded by rpmB.

13. The antisense morpholino oligomer of claim 11, where the ribosomal protein is a 30S ribosomal protein encoded by rpsJ.

14. The antisense morpholino oligomer of claim 1, where the protein associated with a biochemical pathway and/or cellular process is a cell division protein that assembles into a ring at the future site of the septum of bacterial cell division encoded by ftsZ.

15. The antisense morpholino oligomer of claim 1, where the protein associated with a biochemical pathway and/or cellular process is a DNA or chromosomal replication protein encoded by dnaB.

16. The antisense morpholino oligomer of claim 1, where the protein associated with a biochemical pathway and/or cellular process is a lipopolysaccharide biosynthesis protein encoded by lpxC.

17. The antisense morpholino oligomer of claim 1, where the targeting sequence is set forth in SEQ ID NOS: 4-11, comprises a fragment of at least 10 contiguous nucleotides of SEQ ID NOS: 4-11, or comprises a variant having at least 80% sequence identity to SEQ ID NOS: 4-11, where thymine bases (T) are optionally uracil bases (U).

18. The antisense morpholino oligomer of claim 17, which is selected from Table 2B.

19. The antisense morpholino oligomer of claim 2, where T is selected from:

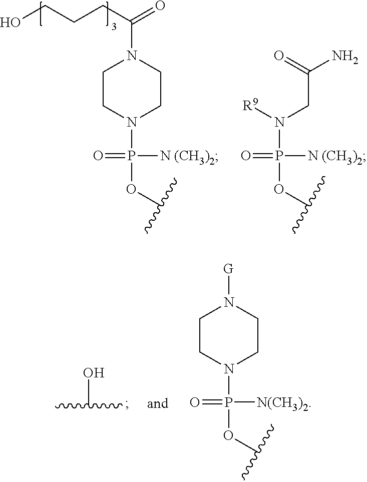

20. The antisense morpholino oligomer of claim 2, where R² is selected from H, G, acyl, trityl, 4-methoxytrityl, benzoyl, and stearoyl.

21. The antisense morpholino oligomer of claim 2, where T is selected from: 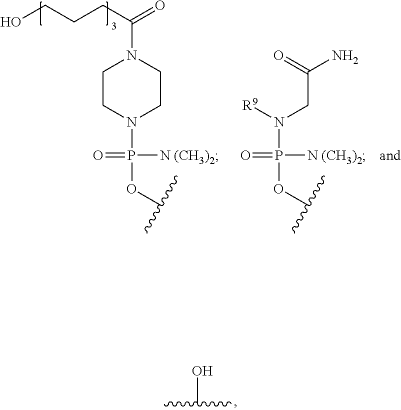
and
R² is G.

22. The antisense morpholino oligomer of claim 2, where T is of the formula: 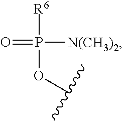
R⁶ is of the formula: 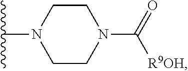
and R² is G.

23. The antisense morpholino oligomer of claim 2, where T is of the formula: 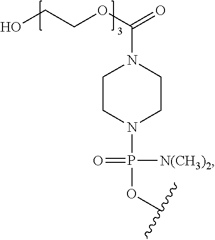
and R² is G.

24. The antisense morpholino oligomer of claim 2, where T is of the formula:

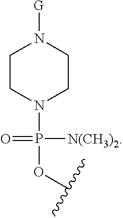

25. The antisense morpholino oligomer of claim 24, where R² is selected from H, acyl, trityl, 4-methoxytrityl, benzoyl, and stearoyl.

26. The antisense morpholino oligomer of claim 2, where at least one instance of R¹ is —N(CH₃)₂.

27. The antisense morpholino oligomer of claim 26, where each R¹ is —N(CH₃)₂.

28. The antisense morpholino oligomer of claim 1, where the CPP is selected from: 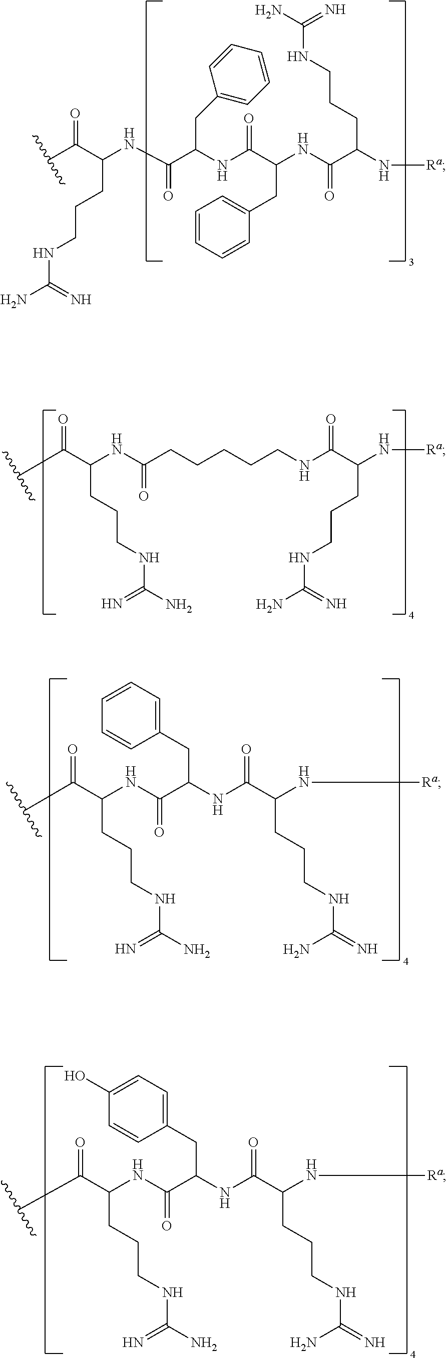 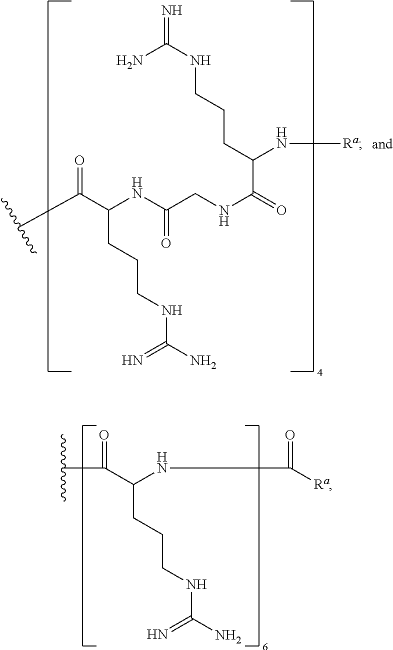
where R^{a} is selected from H, acetyl, benzoyl, and stearoyl.

29. The antisense morpholino oligomer of claim 2, where G is selected from: 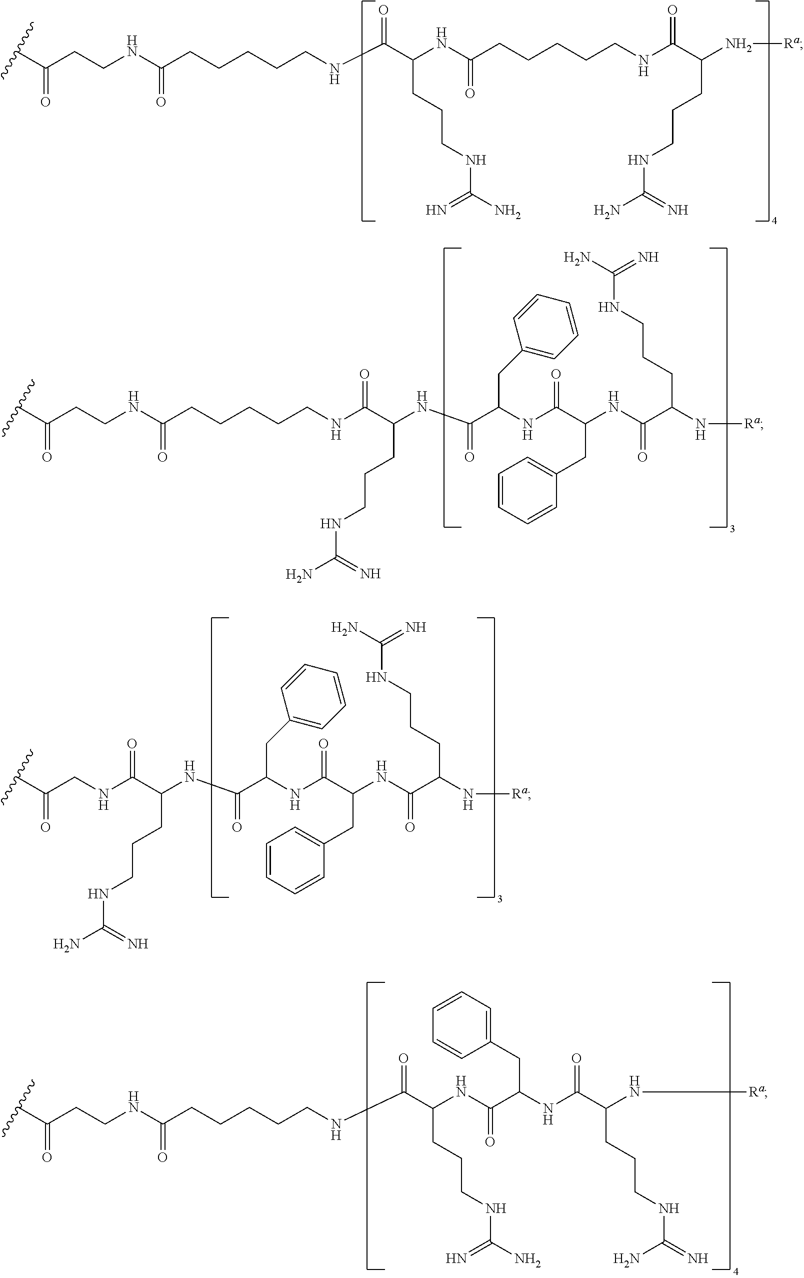 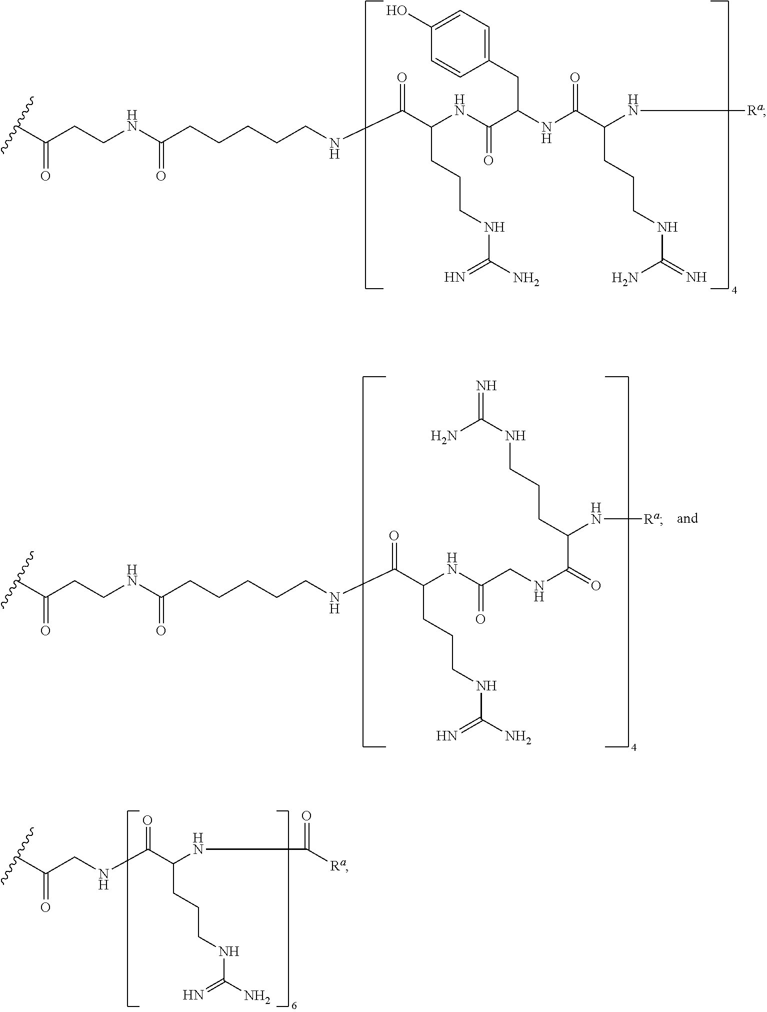
where R^{a} is selected from H, acetyl, benzoyl, and stearoyl.

30. The antisense morpholino oligomer of claim 1, where the antisense oligomer is of the formula (VII) selected from: 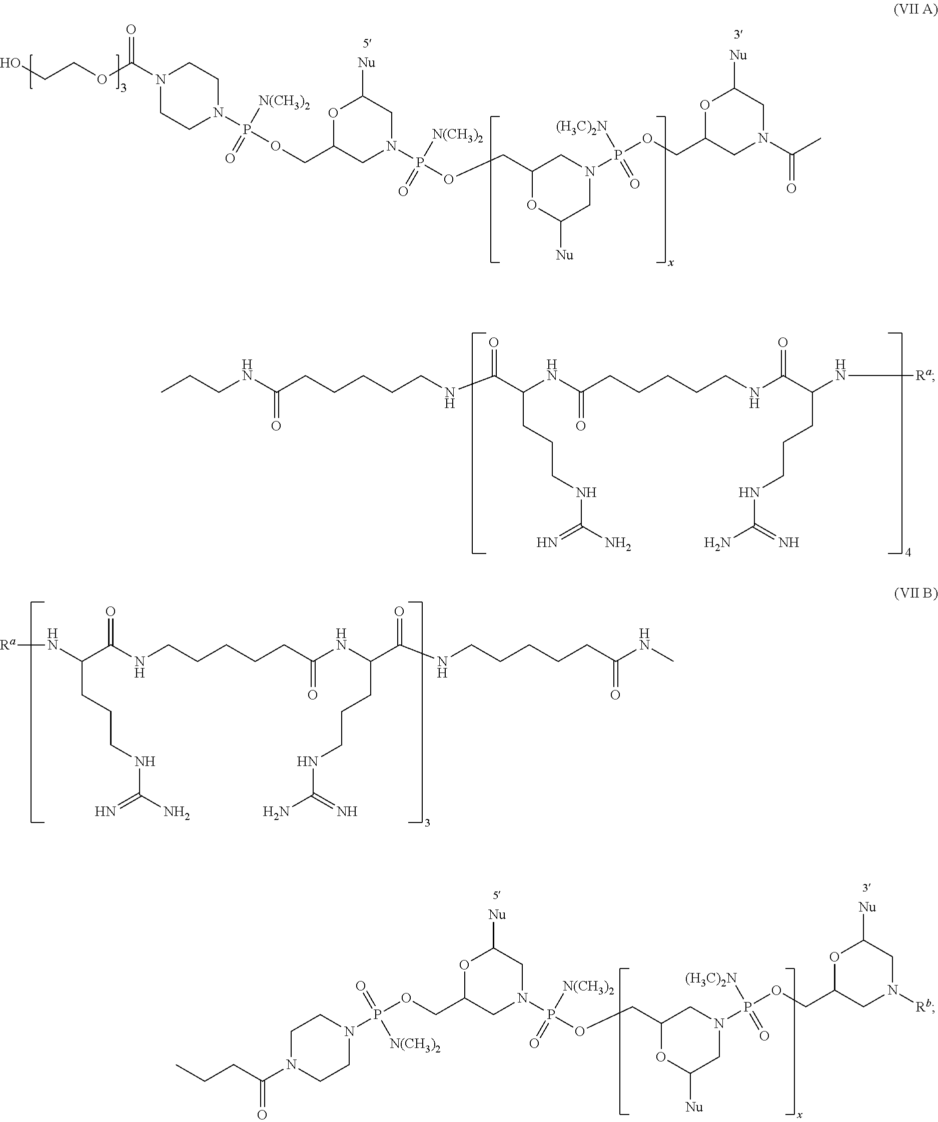 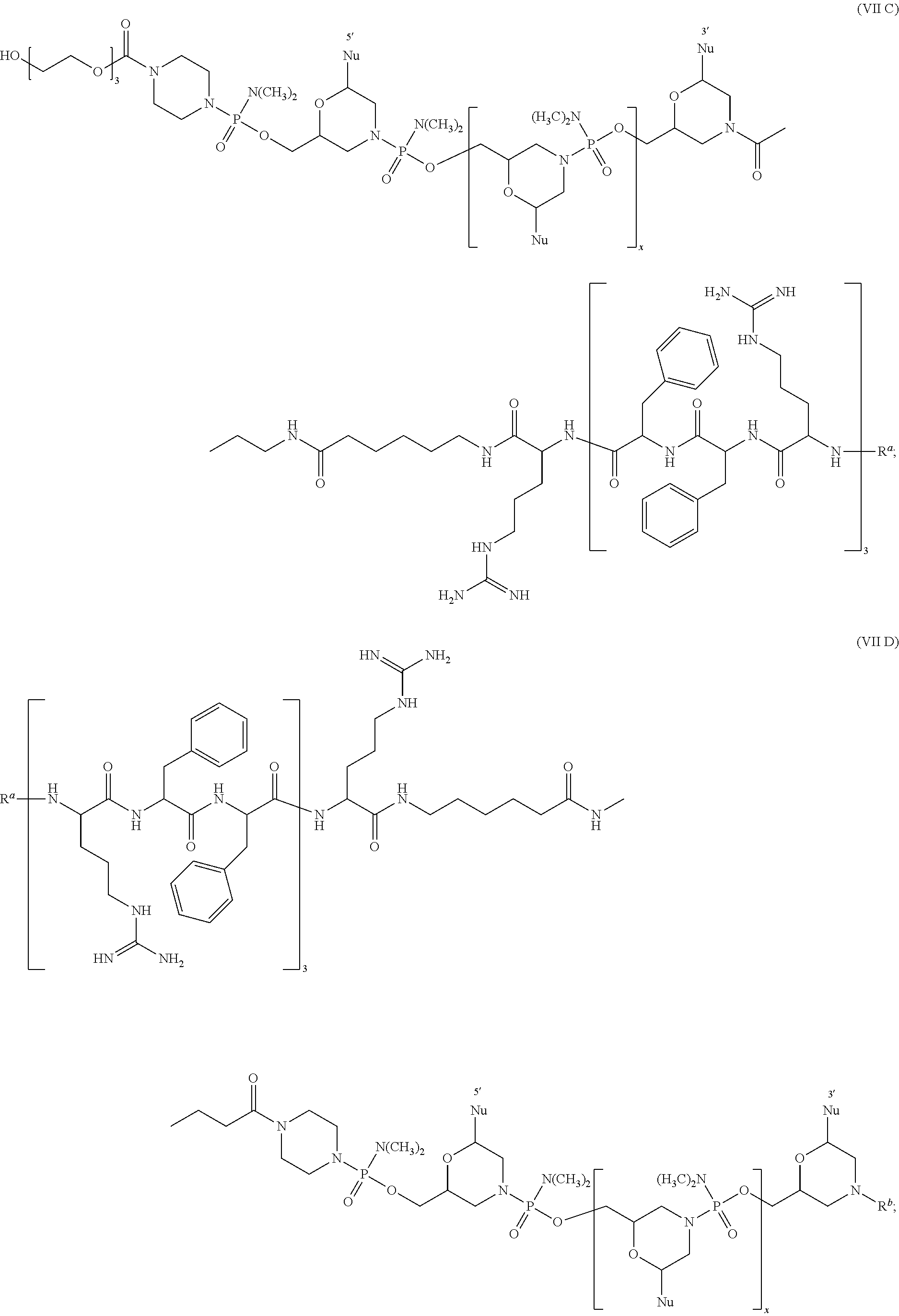 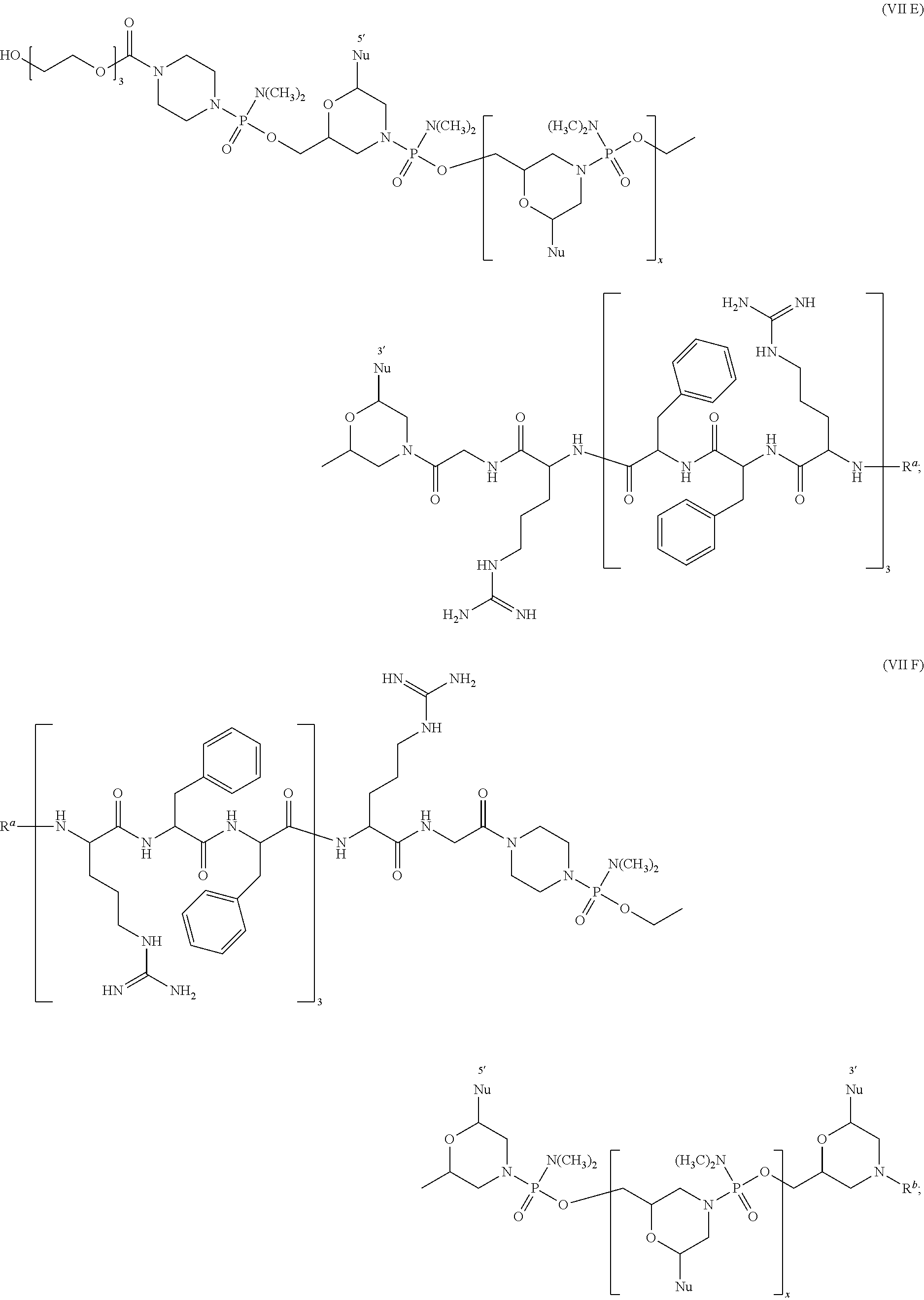 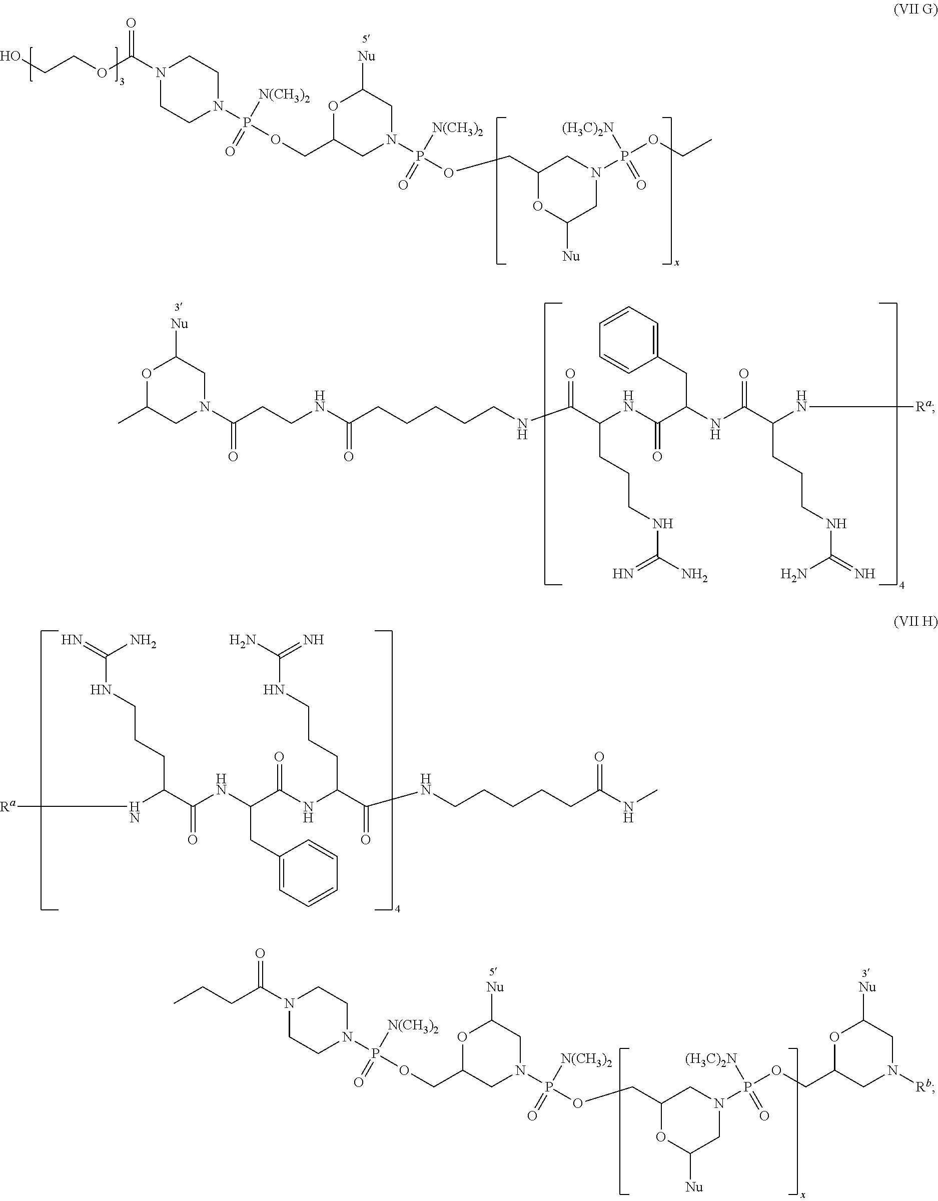 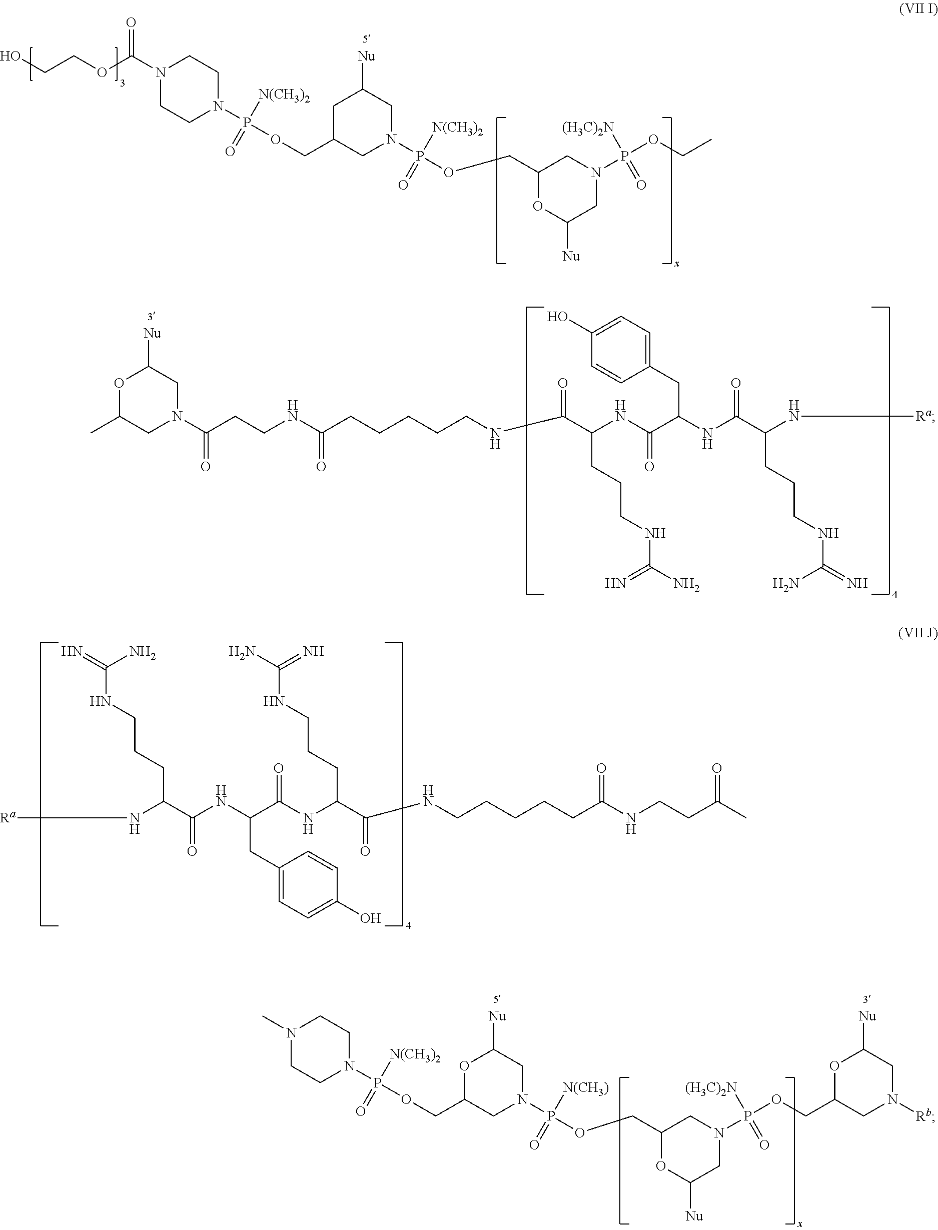 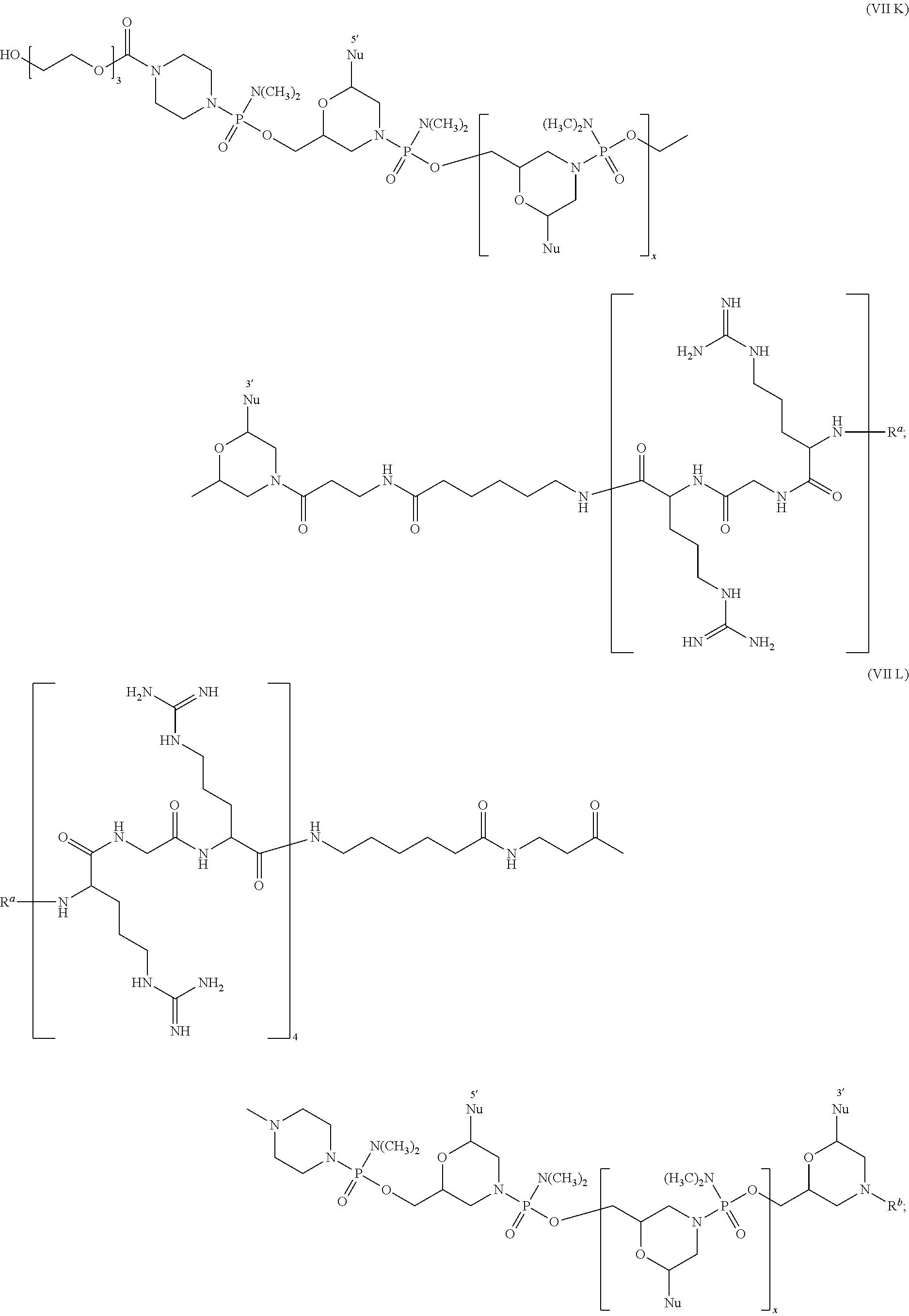 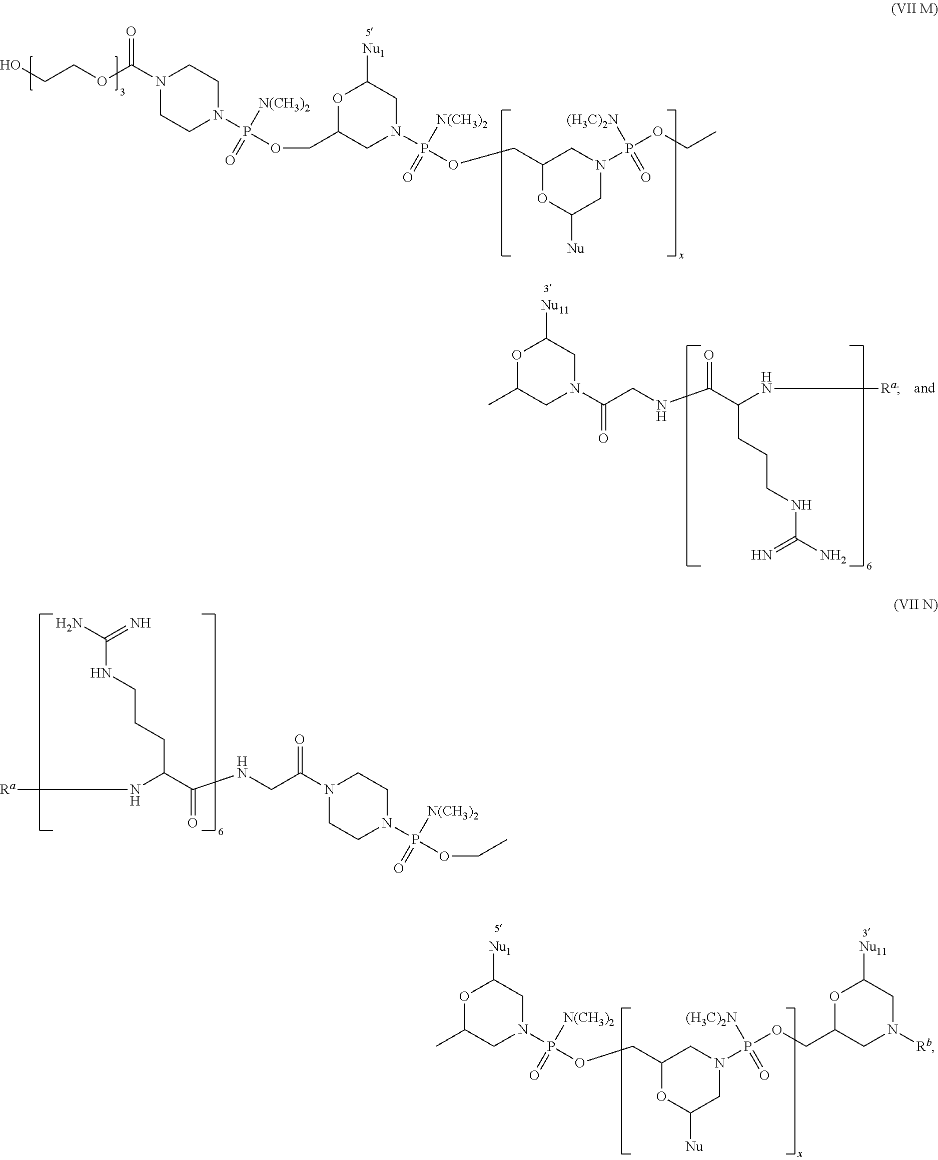
or a pharmaceutically acceptable salt of any of the foregoing,
where R^{a} is selected from H, acetyl, benzoyl, and stearoyl, R^{b} is selected from H, acetyl, benzoyl, stearoyl, trityl, and 4-methoxytrityl, and X and Nu are as defined in claim 1.

31. The antisense morpholino oligomer of claim 30, where R^{a} is acetyl and R^{b} is H.

32. The antisense morpholino oligomer of claim 2, where the targeting sequence is selected from:
SEQ ID NO: 1
(TGCTCATACTC);
b)
SEQ ID NO: 2
(CTTCGATAGTG);
c)
SEQ ID NO: 3
(CGTTTCATTAA));
where X is 9, and where thymine bases (T) may be uracil bases (U).

33. The antisense morpholino oligomer of claim 2, where the targeting sequence is selected from:
a)
SEQ ID NO: 4
(GTCTATTCTCC);
b)
SEQ ID NO: 5
(GACATGTCTAT);
c)
SEQ ID NO: 6
(TGGTTCTGCAT);
d)
SEQ ID NO: 7
(TTTATCCATTG);
e)
SEQ ID NO: 8
(GTTCAAACATA);
f)
SEQ ID NO: 9
(AGTTTCTCTCC);
g)
SEQ ID NO: 10
(TTCCTGCCATA);
h)
SEQ ID NO: 11
(TTTGATCATCG);
where X is 9, and where thymine bases (T) may be uracil bases (U).

34. A pharmaceutical composition, comprising a pharmaceutically acceptable carrier and an antisense morpholino oligomer, optionally of claim 1, wherein the antisense morpholino oligomer is composed of morpholino subunits and phosphorus-containing intersubunit linkages joining a morpholino nitrogen of one subunit to a 5′-exocyclic carbon of an adjacent subunit, and having (a) about 10-40 nucleotide bases, and (b) a targeting sequence of sufficient length and complementarity to specifically hybridize to a bacterial mRNA target sequence that encodes a protein associated with a biochemical pathway and/or cellular process, where the oligomer is conjugated to a cell-penetrating peptide (CPP).

35. The pharmaceutical composition of claim 34, wherein the antisense morpholino oligomer is of formula (I): 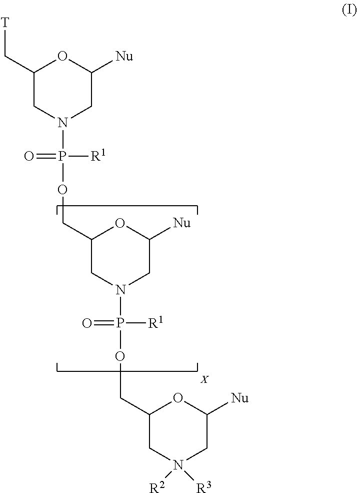
or a pharmaceutically acceptable salt thereof,
where each Nu is a nucleobase which taken together forms a targeting sequence;
X is an integer from 9 to 38;
T is selected from OH and a moiety of the formula: 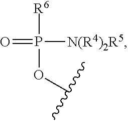
where each R⁴ is independently C₁-C₆ alkyl, and R⁵ is selected from an electron pair and H, and R⁶ is selected from OH, —N(R⁷)CH₂C(O)NH₂, and a moiety of the formula: 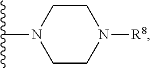
where:
R⁷ is selected from H and C₁-C₆ alkyl, and
R⁸ is selected from G, —C(O)—R⁹OH, acyl, trityl, and 4-methoxytrityl, where:
R⁹ is of the formula —(O-alkyl)_{y}- where y is an integer from 3 to 10 and each
of the y alkyl groups is independently selected from C₂-C₆ alkyl;
each instance of R¹ is —N(R¹⁰)₂R¹¹ where each R¹⁰ is independently C₁-C₆ alkyl, and R¹¹
is selected from an electron pair and H;
R² is selected from H, G, acyl, trityl, 4-methoxytrityl, benzoyl, stearoyl, and a moiety of the formula: 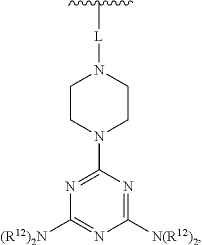
where L is selected from —C(O)(CH₂)₆C(O)— and —C(O)(CH₂)₂S₂(CH₂)₂C(O)—, and each R¹² is of the formula —(CH₂)₂OC(O)N(R¹⁴)₂ where each R¹⁴ is of the formula —(CH₂)₆NHC(═NH)NH₂; and
R³ is selected from an electron pair, H, and C₁-C₆ alkyl,
where G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,
and —C(O)CH₂NH—CPP, or G is of the formula: 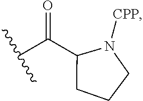
where the CPP is attached to the linker moiety by an amide bond at the CPP carboxy terminus, with the proviso that only one instance of G is present,
where the targeting sequence specifically hybridizes to a bacterial mRNA target sequence that encodes a protein associated with a biochemical pathway and/or cellular process.

36. The pharmaceutical composition of claim 34, wherein the targeting sequence specifically hybridizes to a Klebsiella pneumonia mRNA target sequence that encodes a protein associated with a biochemical pathway and/or cellular process.

37. A method of reducing expression and activity of a protein associated with a biochemical pathway and/or cellular process in a bacterium, comprising contacting the bacterium with an antisense morpholino oligomer, optionally of claim 1, wherein the antisense morpholino oligomer is composed of morpholino subunits and phosphorus-containing intersubunit linkages joining a morpholino nitrogen of one subunit to a 5′-exocyclic carbon of an adjacent subunit, and having (a) about 10-40 nucleotide bases, and (b) a targeting sequence of sufficient length and complementarity to specifically hybridize to a bacterial mRNA target sequence that encodes a protein associated with a biochemical pathway and/or cellular process, where the oligomer is conjugated to a cell-penetrating peptide (CPP).

38. The method of claim 37, wherein the antisense morpholino oligomer is of formula (I): 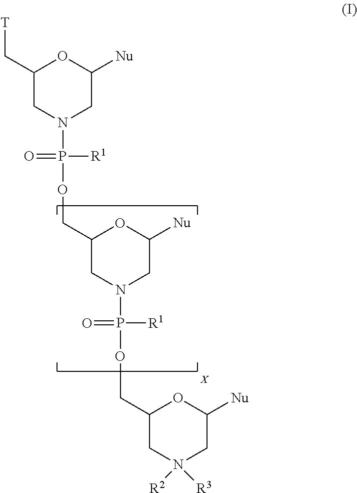
or a pharmaceutically acceptable salt thereof,
where each Nu is a nucleobase which taken together forms a targeting sequence;
X is an integer from 9 to 38;
T is selected from OH and a moiety of the formula: 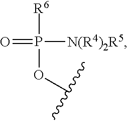
where each R⁴ is independently C₁-C₆ alkyl, and R⁵ is selected from an electron pair and H, and R⁶ is selected from OH, —N(R⁷)CH₂C(O)NH₂, and a moiety of the formula: 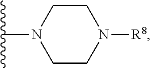
where:
R⁷ is selected from H and C₁-C₆ alkyl, and
R⁸ is selected from G, —C(O)—R⁹OH, acyl, trityl, and 4-methoxytrityl, where:
R⁹ is of the formula —(O-alkyl)_{y}- where y is an integer from 3 to 10 and each
of the y alkyl groups is independently selected from C₂-C₆ alkyl;
each instance of R¹ is —N(R¹⁰)₂R¹¹ where each R¹⁰ is independently C₁-C₆ alkyl, and R¹¹
is selected from an electron pair and H;
R² is selected from H, G, acyl, trityl, 4-methoxytrityl, benzoyl, stearoyl, and a moiety of the formula: 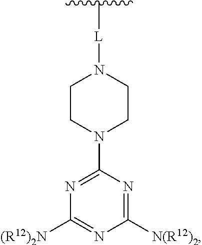
where L is selected from —C(O)(CH₂)₆C(O)— and —C(O)(CH₂)₂S₂(CH₂)₂C(O)—, and each R¹² is of the formula —(CH₂)₂OC(O)N(R¹⁴)₂ where each R¹⁴ is of the formula —(CH₂)₆NHC(═NH)NH₂; and
R³ is selected from an electron pair, H, and C₁-C₆ alkyl,
where G is a cell penetrating peptide (“CPP”) and linker moiety selected from —C(O)(CH₂)₅NH—CPP, —C(O)(CH₂)₂NH—CPP, —C(O)(CH₂)₂NHC(O)(CH₂)₅NH—CPP,
and —C(O)CH₂NH—CPP, or G is of the formula: 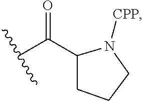
where the CPP is attached to the linker moiety by an amide bond at the CPP carboxy terminus, with the proviso that only one instance of G is present,
where the targeting sequence specifically hybridizes to a bacterial mRNA target sequence that encodes a protein associated with a biochemical pathway and/or cellular process.

39. The method of claim 37, wherein the targeting sequence specifically hybridizes to a Klebsiella pneumonia mRNA target sequence that encodes a protein associated with a biochemical pathway and/or cellular process.

40. The method of claim 37, where the bacterium is in a subject, and the method comprises administering the antisense oligomer to the subject in need thereof.

41. The method of claim 37, where the bacterium is from the genera Klebsiella.

42. The method of claim 37, where the bacterium is selected from an antibiotic-resistant strain of Klebsiella and a multi-drug resistant (MDR) strain of Klebsiella.

43. The method of claim 37, where the bacterium is Klebsiella pneumoniae, optionally selected from a strain in Table 3.

44. The method of claim 40, wherein the subject in need thereof is immunocompromised, and/or has an underlying lung disease, optionally cystic fibrosis (CF) and/or chronic granulomatous disease (CGD).

45. The method of claim 37, wherein the bacterium is in the lung(s) of the subject, as a bacterial lung infection.

46. The method of claim 45, wherein administration of the antisense oligomer reduces bacterial lung burden by at least about 10%.

47. The method of claim 40, wherein the bacterium has formed or is at risk for forming a biofilm in the subject.

48. The method of claim 47, wherein administration of the antisense oligomer reduces biofilm formation or existing biofilm by at least about 10%.

49. The method of claim 37, comprising administering the oligomer separately or concurrently with an antimicrobial agent, optionally where administration of the oligomer increases susceptibility of the bacterium to the antimicrobial agent.

50. The method of 49, where the antimicrobial agent is selected from one or more of a β-lactam antibiotic, an aminoglycoside antibiotic, and a polymyxin.

51. The method of claim 49, where the combination of oligomer and the antimicrobial agent increases the susceptibility of the bacterium to the antibiotic relative to the oligomer and/or the microbial agent alone.

52. The method of claim 40, wherein the subject is suspected of having or has a urinary tract infection.

53. The method of claim 40, wherein the subject is suspected of having a Klebsiella spp. or E. coli infection.

54. The method of claim 52, wherein the antisense morpholino oligomer comprises SEQ ID NO: 1 or 2.
